SEQUENCE LISTING (16 sequences)

SEQ ID NO: 1 - protein, 5 aa, Unknown
  source: /note=“Description of Unknown Laminin B1 peptide”
YIGSR

SEQ ID NO: 2 - protein, 4 aa, Unknown
  source: /note=“Description of Unknown Fibronectin peptide”
RGDS

SEQ ID NO: 3 - protein, 4 aa, Unknown
  source: /note=“Description of Unknown Fibronectin peptide”
REDV

SEQ ID NO: 4 - protein, 4 aa, Unknown
  source: /note=“Description of Unknown Vitronectin peptide”
RGDV

SEQ ID NO: 5 - protein, 5 aa, Unknown
  source: /note=“Description of Unknown Laminin A peptide”
LRGDN

SEQ ID NO: 6 - protein, 5 aa, Unknown
  source: /note=“Description of Unknown Laminin A peptide”
IKVAV

SEQ ID NO: 7 - protein, 5 aa, Unknown
  source: /note=“Description of Unknown Laminin B1 peptide”
PDSGR

SEQ ID NO: 8 - protein, 10 aa, Unknown
  source: /note=“Description of Unknown Laminin B2 peptide”
RNIAEIIKDA

SEQ ID NO: 9 - protein, 4 aa, Unknown
  source: /note=“Description of Unknown Collagen 1 peptide”
RGDT

SEQ ID NO: 10 - protein, 4 aa, Unknown
  source: /note=“Description of Unknown Collagen 1 peptide”
DGEA

SEQ ID NO: 11 - protein, 6 aa, Unknown
  source: /note=“Description of Unknown Fibronectin peptide”
PRRARV

SEQ ID NO: 12 - protein, 19 aa, Unknown
  source: /note=“Description of Unknown Fibronectin peptide”
YEKPGSPPREVVPRPRPGV

SEQ ID NO: 13 - protein, 28 aa, Unknown
  source: /note=“Description of Unknown Vitronectin peptide”
RPSLAKKQRFRHRNRKGYRSQRGHSRGR

SEQ ID NO: 14 - protein, 17 aa, Unknown
  source: /note=“Description of Unknown Laminin peptide”
RIQNLLKITNLRIKFVK

SEQ ID NO: 15 - protein, 5 aa, Unknown
  source: /note=“Description of Unknown Recombinant fibronectin fragment peptide”
FNIII

SEQ ID NO: 16 - protein, 16 aa, Unknown
  source: /note=“Description of Unknown MMP substrate peptide”
GCRDGPQGIWGQDRCG

(19) United States
(12) Patent Application Publication
(10) Pub. No.: US 2011/0293666 A1
(43) Pub. Date: Dec. 1, 2011

(54) Bioengineered Tissue Constructs and Methods for Production and Use

(76) Inventors: Xianyan Wang, Acton, MA (US); Katherine C. Faria, Middlebro, MA (US)

(21) Appl. No.: 13/007,201

(22) Filed: Jan. 14, 2011

Related U.S. Application Data
(63) Continuation-in-part of application No. 12/324,367, filed on Nov. 26, 2008.
(60) Provisional application No. 60/990,757, filed on Nov. 28, 2007.
(60) Provisional application No. 61/021,176, filed on Jan. 15, 2008.
(60) Provisional application No. 61/295,073, filed on Jan. 14, 2010.
(60) Provisional application No. 61/337,938, filed on Feb. 12, 2010.
(60) Provisional application No. 61/347,725, filed on May 24, 2010.

(51) Int. Cl. A61K 9/00; A61K 35/12; A61K 35/36; A61K 35/44; A61K 35/42; A61K 35/22; A61K 35/38; C12N 5/071; A61K 35/32

(52) U.S. Cl. 424/400; 435/325; 424/93.7

(57) ABSTRACT

Bioengineered constructs are formed from cultured cells induced to synthesize and secrete endogenously produced extracellular matrix components, e.g., without the requirement of exogenous matrix components or network support or scaffold members. The bioengineered constructs of the invention can be treated in various ways such that the cells of the bioengineered constructs can be devitalized and/or removed without compromising the structural integrity of the constructs. Moreover, the bioengineered constructs of the invention can be used in conjunction with biocompatible/bioremodelable solutions that allow for various geometric configurations of the constructs.

### RELATED APPLICATIONS

This application is a continuation-in-part of U.S. patent application Ser. No. 12/324,367, filed on Nov. 26, 2008, which claims the benefit of U.S. Provisional Application Ser. No. 60/990,757, filed on Nov. 28, 2007 and of U.S. Provisional Application Ser. No. 61/021,176, filed on Jan. 15, 2008, as well as the benefit of priority to U.S. Provisional Application No. 61/347,725, filed on May 24, 2010, U.S. Provisional Application No. 61/337,938, filed on Feb. 12, 2010, and U.S. Provisional Application No. 61/295,073, filed on Jan. 14, 2010; the entire contents of each of the applications is incorporated herein by reference.

### FIELD OF THE INVENTION

The invention is in the field of tissue engineering. This invention is directed to a method for producing a bioengineered construct. The bioengineered constructs are biocompatible and bioremodelable and can be used for clinical purposes.

### BACKGROUND OF THE INVENTION

Bone, cartilage, tendon, ligament, muscle, adipose, and marrow stroma are examples of mesenchymal tissues (i.e., tissues that differentiate from mesenchymal stem cells).

Mesenchymal tissue may be injured during surgery (e.g., gastric bypass; colostomies; stomach and large and small bowel resections; vascular grafts; vascular implants; coronary artery bypass grafts; tummy tucks; abdominal surgeries; C-sections; trachea implant sites; catheter implant sites; sealing of pericardium, pleura, and dura trauma); or develop from a genetic disease or environmental perturbation (e.g., vulnerable plaque; abdominal aortic/aneurysm ruptures; stomach or small intestine ulcer perforations; Crohn's disease; inflammatory bowel disease, urinary incontinence; nose/septum repairs; anal fistulas; ostomies; muscle tears; cartilage tears; joint coating material; soft tissue and muscle wall hernia repairs; corneal implants, bone grafts and repairs; and cardiovascular regeneration, thereby generating wounds.

Accordingly, new therapies for repairing mesenchymal tissues are needed.

### SUMMARY OF THE INVENTION

Featured herein are bioengineered constructs comprising extracellular matrix (ECM) in forms., which are optimized for particular therapeutic uses. Certain constructs are comprised of extracellular matrix produced by cultured fibroblasts and/or mesenchymal stem cells (MSCs). Certain constructs also comprise the cells that produce the matrix. In certain constructs, the cells have been devitalized. In other constructs, the cells, which produce the extracellular matrix have been removed to produce decellularized constructs.

Certain constructs have a thickness of at least about 30 μm. Certain constructs include pores having an average diameter in the range of 10 to 100 um. Certain constructs have an average Fmax of at least 0.4 Newtons. Certain constructs have an ultimate tensile strength (UTS) of at least 0.4 Megapascals. Certain constructs have a plastic deformation tolerance of at least 0.4 times the initial length.

The ECM in the constructs may be further processed (e.g., dehydrated, crosslinked, contracted, micronized, sterilized, etc.) or further combined with other biologically active substances or support materials (e.g., silk, an adhesive, etc.) for preparation of therapeutic products.

Further featured are methods for making and modifying the bioengineered constructs, including methods to control construct thickness, pore size, and composition.

The bioengineered constructs described herein can be administered to subjects to enhance the vitality, growth and/or repair of soft tissue, including for treatment of chronic or acute wounds.

Other features and advantages will become apparent from the following detailed description and claims.

### BRIEF DESCRIPTION OF THE DRAWINGS

FIGS. 1A-1B show a time course analysis of extracellular matrix formation rate by MSCs between days 5 and 12 (FIG. 1A) or between days 12 and 18 (FIG. 1B). n=9 (3 independent constructs per group with 3 measurements per construct). A trend line and slope equation are shown.

FIG. 2 shows a correlation between increasing bioengineered construct thickness as a function of increased TGF-alpha concentration. No TGF-alpha: 0 ng/mL; 1.5×: 30 ng/mL TGF-alpha; 5×: 100 ng/mL TGF-alpha; and 10×: 200 ng/mL TGF-alpha. n=9 (3 independent constructs per group with 3 measurements per construct), except for 1.5× and 10× where n=6 (2 independent constructs per group and 3 measurements per construct).

FIG. 3 shows a correlation between decreasing bioengineered construct thickness as a function of increased Prostaglandin 2 (PGE₂) concentration having a constant amount of 20 ng/mL TGF-alpha. No PGE₂: 0 ng/mL; 5×: 19 ng/mL PGE₂; 10×: 38 ng/mL PGE₂; and 50×: 190 ng/mL PGE₂. n=9 (3 independent constructs per group with 3 measurements per construct).

FIG. 4 shows a correlation between increasing bioengineered construct thickness as a function of increased TGF-alpha concentration and cell seeding density across bioengineered constructs derived from MSCs of different cell types (HDF: neonatal human dermal fibroblasts; HUCPVC: human umbilical cord perivascular cells; BM-MSC: bone marrow derived mesenchymal stem cells; and Pre-Adipo: pre-adipocytes). Chemically defined cell culture media described in Example 1 was used (e.g., 200 ng/mL TGF-alpha) and seeding densities were 30×10⁶ cells per 75 mm insert, which is equivalent to 9.6×10⁶ cells per 24 mm insert. Matrix thickness measurements collected from hematoxylin and eosin stained sections fixed after 18 days in culture. Bars (mean±S.D, n=12) represent the average thickness of n=3 independent constructs imaged in 4 separate locations.

FIGS. 5A-5B show representative hematoxylin and eosin stained, Masson's Trichome/Goldner (MTG) stained, and SEM sections of bioengineered constructs derived from MSCs of different cell types (HDF: neonatal human dermal fibroblasts; HUCPVC: human umbilical cord perivascular cells; BM-MSC: bone marrow derived mesenchymal stem cells; and Pre-Adipo: pre-adipocytes) after 18 days in culture. Chemically defined cell culture media described in Example 1 was used (e.g., 200 ng/mL TGF-alpha) and seeding densities were 30×10⁶ cells per 75 mm insert, which is equivalent to 9.6×10⁶ cells per 24 mm insert. Images captured at 20× magnification.

FIGS. 6A-6C show representative Fmax, ultimate tensile strength (UTS), and modulus of elasticity properties of bioengineered constructs derived from MSCs of different cell types (HDF-02: neonatal human dermal fibroblasts; HUC-02: human umbilical cord perivascular cells; MSC-02: bone marrow derived mesenchymal stem cells; and PAD-02: pre-adipocytes) after 18 days in culture. Chemically defined cell culture media described in Example 1 was used (e.g., 200 ng/mL TGF-alpha) and seeding densities were 30×10⁶ cells per 75 mm insert, which is equivalent to 9.6×10⁶ cells per 24 mm insert. Bars (mean±S.D, n=9) represent the average Fmax, UTS, modulus of elasticity of 3 independent constructs each tested 3 times.

FIGS. 7A-7B show a summary of differences in extracellular matrix and adhesion components (FIG. 7A; 17 upregulated genes>2-fold in HUCPVC-derived relative to HDF-derived bioengineered constructs) and growth factors (FIG. 7B; 8 upregulated genes>2-fold in HUCPVC-derived relative to HDF-derived bioengineered constructs) between HUCPVC-derived and HDF-derived bioengineered constructs.

FIGS. 8A-8D show results of a time-course comparison of IL-6, IL-8, and VEGF levels within the conditioned media generated by various MSC-derived and HDF-derived bioengineered constructs resulting from CBA analyses. The mean and standard deviations are calculated from an average of n=3 conditioned media samples. Quantification of HA levels resulting from ELISA analyses is also shown.

FIG. 9 shows results of a cellular migration assay. An indirect 2-D Migration assay comparing closure index as a function of conditioned media collected from various embodiments. Assay is performed on keratinocytes cultured in conditioned media collected from HDF-02 and HUCPVC VCT-02 units at Day 5 and Day 18. The figure consists of representative bright field images of the keratinocytes stained with Acid Fuschin dye after 24 hours of induction in the conditioned media as well as a graphical representation of the closure index values indicating the maximum closure in the HUCPVC VCT-02 Day 5 conditioned media samples.

FIGS. 10A-10C show results of multilineage potential assays conducted on MSC-derived (HUC-02) and HDF-derived (HDF-02) bioengineered constructs and cells isolated therefrom. FIG. 10A shows gene expression data from cells within bioengineered constructs induced using osteogenic induction media using a panel of osteogenic genes. FIG. 10B shows gene expression data from cells isolated from bioengineered constructs induced using osteogenic induction media using a panel of osteogenic genes. FIG. 10C shows Oil Red O staining results from cells within bioengineered constructs induced using adipogenic induction media.

FIGS. 11A-11E show representative histological sections and quantitation of alpha-smooth muscle actin (αSMA) staining from a 100% MSC-derived bioengineered constructs (FIG. 11A), 50% HUCPVC-50% HDF-derived bioengineered constructs (FIG. 11B), 10% HUCPVC-90% HDF-derived bioengineered constructs (FIG. 11C), and 100% HDF-derived bioengineered constructs (FIG. 11D) after 1 week of subcutaneous implantation into nude mice. Dark areas denote positive staining for αSMA. FIG. 11E shows blood vessel quantification within implant area as determined by αSMA positive staining A total of two animals per group (n=2) were used for the analysis. The number of αSMA positive vessels was manually counted using 40× objective on a microscope. The number of positive vessels was then normalized to the implant area.

FIG. 12 shows independent histological images of bioengineered constructs that have been formalin-fixed immediately after culture.

FIG. 13 shows independent histological images of bioengineered constructs that have been allowed to undergo controlled contraction prior to formalin-fixation.

FIGS. 14A-14G show results of controlling pore sizes within extracellular matrices of bioengineered constructs. FIG. 14A shows the different uses of bioengineered constructs according to different average pore diameter properties. FIG. 14B shows quantitative analysis of average pore diameters and standard deviations from bioengineered constructs controlled contracted, lyophilized at a final freezing temperatures of −40° C. at a rate of 0.1° C. per minute, and either not crosslinked, crosslinked with EDC, or crosslinked using DHT methods. FIG. 14C shows a representative histological section quantified in FIG. 14C. FIG. 14D shows a representative histological section of a bioengineered construct ramped to a final freezing temperatures of −10° C. at a rate of 0.5° C. per minute. FIG. 14E shows representative histological sections of bioengineered constructs control contracted and subsequently either air dried (top panel) or lyophilized at a final freezing temperature of −40° C. (bottom panel). FIG. 14F shows MSC-derived bioengineered constructs naturally having pores, whereas FIG. 14G shows that such average pore diameter can be increased by lyophilizing.

FIGS. 15A-15E shows the effects on biophysical properties of bioengineered constructs resulting from supplementing chemically defined cultured media with bFGF. FIG. 15A shows that bFGF supplementation reduces bioengineered construct thickness. FIG. 15B shows the results of bFGF dose response analysis in which sub types of collagen accumulation decreased as bFGF supplementation increased. FIG. 15C shows relative levels of both acid- and pepsin-soluble collagen (black) relative to total collagen and other collagen (grey). Sulfated glycosaminoglycan (sGAG; FIG. 15D) and hyaluronic acid (HA; FIG. 15E) accumulated to lower levels in bFGF-supplemented bioengineered constructs relative to controls.

FIG. 16 shows human dermal fibroblasts that have migrated through porous silk scaffolds and are uniformly disposed throughout the silk scaffold.

FIGS. 17A-17D show stained human umbilical vein endothelial cells atop porous silk scaffolds with devitalized human dermal fibroblasts and its corresponding extracellular matrix, in vitro. An in vitro angiogenesis assay was developed by examining alignment of stained HUVECs on silk scaffold embodiments. HUVECs were cultured on the silk scaffolds for 11 days and fluorescence images were captured. HUVEC alignment is not visible on the silk scaffold (FIG. 17A) or the silk scaffold pre-conditioned in matrix media (FIG. 17B), but it is prominent in the silk scaffold with living human dermal fibroblasts (HDF) (FIG. 17C) and the silk scaffold with devitalized HDFs (FIG. 17D).

### DETAILED DESCRIPTION OF THE INVENTION

Engineered living tissue constructs rely on the addition or incorporation of exogenous matrix components or synthetic members for structure or support, or both.

The bioengineered tissue constructs described herein exhibit many of the native features of the tissue from which cells are derived. The tissue constructs thus produced can, for example, be grafted into a subject.

In one embodiment is featured a cell-matrix construct comprising a first cell type and an endogenously produced extracellular matrix, wherein the first cell type is capable of synthesizing and secreting extracellular matrix.

In another embodiment is featured a bilayer construct comprising a first cell type and endogenously produced extracellular matrix and a layer of cells of a second type disposed thereon or within the cell-matrix construct formed by the first cell type.

Also featured is a cell-matrix construct comprising fibroblasts, such as those derived from dermis, to form a cultured dermal construct.

Further featured is a cell-matrix construct comprising fibroblasts, such as those derived from dermis, to form a cultured dermal construct with a layer of keratinocytes cultured thereon to form an epidermal layer to result in a cultured bilayer skin construct. The cultured skin constructs of the invention express many physical, morphological, and biochemical features of native skin.

The cell-matrix construct can be a tissue construct that is similar to the dermal layer of skin, a human dermal construct, that is formed in a defined system comprising human-derived cells utilizing no chemically undefined components during culture.

Tissue constructs may be fabricated in a chemically defined system comprising human-derived cells but no chemically undefined or non-human biological components or cells.

Also featured is a structural layer of at least one type of extracellular matrix-producing cell and endogenously produced extracellular matrix components, “matrix”, wherein the matrix is completely cell-synthesized and assembled by culturing the cells. This layer is herein termed a “cell-matrix construct” or a “cell-matrix layer” because the cells secrete and contain themselves within and through their matrix. The cultured tissue constructs do not require, thus do not include, exogenous matrix components, that is, matrix components not produced by the cultured cells but introduced by other means. The cell-matrix construct produced by human dermal fibroblasts is shown to have a predominant concentration of collagen similar to native skin. As evidenced by electron microscopy, the matrix is fibrous in nature comprising collagen that exhibits the quarter-staggered 67 nm banding pattern, as well as packing organization of fibrils and fibril bundles similar to native collagen. Delayed reduction SDS-PAGE has detected the presence of both type I and type III collagen in these constructs, the predominant collagen types found in native human skin. Using standard immunohistochemistry (IHC) techniques, the dermal cell-matrix construct stains positively for decorin, a dermatan sulfate proteoglycan known to be associated with collagen fibrils and believed to regulate fibril diameter in vivo. Decorin can also be visualized in the construct with TEM. The produced tissue also stains positive for tenascin, an extracellular matrix glycoprotein found, for example, in mesenchyme or tissues under repair. Much like tissue under repair in vivo, the tissue produced in culture has been shown to increase its ratio of type I to type III collagen as the matrix is formed. While not wishing to be bound by theory, it is believed that the cells fill in the open space between them quickly with a loose matrix analogous to granulation tissue comprised of mostly type III collagen and fibronectin, and then remodel this loose matrix with a denser matrix comprised of mostly type I collagen. The produced cell-matrix has been shown to contain glycosaminoglycans (GAG), such as hyaluronic acid (HA); fibronectin; proteoglycans besides decorin such as biglycan and versican; and, a profile of sulfated glycosaminoglycans such as di-hyaluronic acid; di-chondroitin-0-sulfate; di-chondroitin-4-sulfate; di-chondroitin-6-sulfate; di-chondroitin-4,6-sulfate; di-chondroitin-4-sulfate-UA-2S; and di-chondroitin-6-sulfate-UA-2S. These structural and biochemical features exhibit themselves as the construct develops in culture and are distinctively evident when the construct approaches its final form. The presence of these components in fully formed cultured dermal cell-matrix construct indicates that the construct has structural and biochemical features approaching that of normal dermis.

While the aforementioned lists biochemical and structural features of a cultured cell-matrix construct formed from dermal fibroblasts, it should be recognized that cultured cell-matrix constructs formed from other types of fibroblasts will produce many of these features and others phenotypes for tissue types from which they originated. In some cases, fibroblasts can be induced to express non-phenotypic components by either chemical exposure or contact, physical stresses, or by transgenic means. Another preferred embodiment of the invention is a cell-matrix layer having a second layer of cells disposed thereon. The second layer of cells is cultured on the cell-matrix layer to form a bioengineered bilayered tissue construct. In a more preferred embodiment, the cells of the second layer are of epithelial origin. The second layer can comprise cultured human keratinocytes that together with a first cell-matrix layer, a cell-matrix construct formed from dermal fibroblasts and endogenous matrix to form a dermal layer, comprise a living skin construct. When fully formed, the epidermal layer is a multilayered, stratified, and well-differentiated layer of keratinocytes that exhibit a basal layer, a suprabasal layer, a granular layer and a stratum corneum. The skin construct has a well-developed basement membrane present at the dermal-epidermal junction as exhibited by transmission electron microscopy (TEM). The basement membrane appears thickest around hemidesmosomes, marked by anchoring fibrils that are comprised of type VII collagen, as visualized by TEM. The anchoring fibrils can be seen exiting from the basement membrane and entrapping the collagen fibrils in the dermal layer. These anchoring fibrils, as well as other basement membrane components, are secreted by keratinocytes. It is also known that while keratinocytes are capable of secreting basement membrane components on their own, a recognizable basement membrane will not form in the absence of fibroblasts. Immunohistochemical staining of the skin construct of the present invention has also shown that laminin, a basement membrane protein is present.

A first cell type and an extracellular matrix-producing cell type can be seeded to a substrate, cultured, and induced to synthesize and secrete an organized extracellular matrix around the cells and thereby form a cell-matrix construct. A surface of the cell-matrix construct can also be seeded with cells of a second cell type and cultured to form a bilayered tissue construct. In a more preferred method, a full thickness skin construct having features similar to native human skin can be formed by culturing fibroblasts, such as human dermal fibroblasts, under conditions sufficient to induce matrix synthesis to form a cell-matrix of dermal cells and matrix, a dermal layer, onto which human epithelial cells, such as keratinocytes, are seeded and cultured under conditions sufficient to form a fully differentiated stratified epidermal layer.

One method of obtaining bioengineered tissue constructs comprises the steps of: (a) culturing at least one extracellular matrix-producing cell type in the absence of exogenous extracellular matrix components or a structural support member; (b) stimulating the cells of step (a) to synthesize, secrete, and organize extracellular matrix components to form a tissue-construct comprised of cells and matrix synthesized by those cells; wherein steps (a) and (b) may be done simultaneously or consecutively; and, (c) devitalizing or decellularizing the tissue construct comprising extracellular matrix components for clinical use. Two or more devitalized or decellularlized tissue constructs may be contacted together and bonded together either by way of crosslinking or the use of a biocompatible or bioresorbable adhesive.

### I. Media Formulations

Cell-matrix constructs may be formed by culturing cells in a culture medium that promotes cell viability, proliferation and synthesis of extracellular matrix components by the cells. Culture medium may be comprised of a nutrient base usually further supplemented with other components. Many commercially available nutrient sources may be useful. These include commercially available nutrient sources which supply inorganic salts, an energy source, amino acids, and B-vitamins such as Dulbecco's Modified Eagle's Medium (DMEM); Minimal Essential Medium (MEM); M199; RPMI 1640; Iscove's Modified Dulbecco's Medium (EDMEM). Minimal Essential Medium (MEM) and M199 require additional supplementation with phospholipid precursors and non-essential amino acids. Commercially available vitamin-rich mixtures that supply additional amino acids, nucleic acids, enzyme cofactors, phospholipid precursors, and inorganic salts include Ham's F-12, Ham's F-10, NCTC 109, and NCTC 135. Albeit in varying concentrations, all basal media provide a basic nutrient source for cells in the form of glucose, amino acids, vitamins, and inorganic ions, together with other basic media components. The most preferred base medium of the invention comprises a nutrient base of either calcium-free or low calcium Dulbecco's Modified Eagle's Medium (DMEM), or, alternatively, DMEM and Ham's F-12 between a 3-to-1 ratio to a 1-to-3 ratio, respectively.

The base medium is supplemented with components such as amino acids, growth factors, and hormones. Certain defined culture media are described in U.S. Pat. No. 5,712,163 to Parenteau, International PCT Publication No. WO 95/31473, and PCT Publication No. WO 00/29553, the disclosures of which are incorporated herein by reference. Other media are known in the art, such as those disclosed in Ham and McKeehan, Methods in Enzymology, 58:44-93 (1979), or for other appropriate chemically defined media, in Bottenstein et al., Methods in Enzymology, 58:94-109 (1979). Base medium can be supplemented with the following components: insulin, transferrin, triiodothyronine (T3), and either or both ethanolamine and o-phosphoryl-ethanolamine, wherein concentrations and substitutions for the supplements may be determined by the skilled artisan.

Culture media formulations may be selected based on the cell types to be cultured and the tissue structure to be produced. The culture medium that is used and the specific culturing conditions needed to promote cell growth, matrix synthesis, and viability will depend on the type of cell, or combinations of types of cells, being grown.

In some instances, such as in the fabrication of bioengineered bilayer skin constructs, the media composition varies with each stage of fabrication as different supplementation is necessary for different purposes. The cell-matrix layer can be formed under defined conditions, that is, cultured in chemically defined media. A tissue construct can comprise a cell-matrix layer provided with a second layer of cells disposed and cultured thereon wherein both cell types are cultured in a defined culture media system. Alternatively, the tissue construct comprises a cell-matrix layer fabricated under defined media conditions and a second layer formed thereon under undefined media conditions. The tissue construct can alternatively comprise a cell-matrix layer fabricated under undefined media conditions and a second layer formed thereon under defined media conditions.

The use of chemically defined culture media is preferred, that is, media free of undefined animal organ or tissue extracts, for example, serum, pituitary extract, hypothalamic extract, placental extract, or embryonic extract or proteins and factors secreted by feeder cells. The media can be free of undefined components and biological components derived from non-human animal sources. Undefined components may also be added at any point in culture. The use of screened human cells cultured using chemically defined components derived from no non-human animal sources results in a defined human tissue construct. Synthetic or recombinant functional equivalents may also be added to supplement chemically defined media. Generally, one of skill in the art of cell culture will be able to determine suitable natural human, human recombinant, or synthetic equivalents to commonly known animal components to supplement the culture media without undue investigation or experimentation. Use of such a constructs minimize the likelihood of adventitious animal or cross-species viral contamination and infection.

Insulin is a polypeptide hormone that promotes the uptake of glucose and amino acids to provide long term benefits over multiple passages. Supplementation of insulin or insulin-like growth factor (IGF) is necessary for long term culture as there will be eventual depletion of the cells' ability to uptake glucose and amino acids and possible degradation of the cell phenotype. Insulin may be derived from either animal, for example bovine, human sources, or by recombinant means as human recombinant insulin. Therefore, a human insulin would qualify as a chemically defined component not derived from a non-human biological source. Insulin supplementation is advisable for serial cultivation and is provided to the media at a wide range of concentrations. A preferred concentration range is between about 0.1 μg/ml to about 500 μg/ml, more preferably at about 5 μg/ml to about 400 μg/ml, and most preferably at about 375 μg/ml. Appropriate concentrations for the supplementation of insulin-like growth factor, such as IGF-1 IGF-2, and the like may be easily determined by one of skill in the art for the cell types chosen for culture.

Transferrin is in the medium for iron transport regulation. Iron is an essential trace element found in serum. As iron can be toxic to cells in its free form, in serum it is supplied to cells bound to transferrin at a concentration range of preferably between about 0.05 to about 50 μg/ml, more preferably at about 5 μg/ml.

Triiodothyronine (T3) is a basic component and is the active form of thyroid hormone that is included in the medium to maintain rates of cell metabolism. Triiodothyronine is supplemented to the medium at a concentration range between about 0 to about 400 ρM, more preferably between about 2 to about 200 ρM and most preferably at about 20 ρM.

Either or both ethanolamine and o-phosphoryl-ethanolamine, which are phospholipids, are added whose function is an important precursor in the inositol pathway and fatty acid metabolism. Supplementation of lipids that are normally found in serum is necessary in a serum-free medium. Ethanolamine and o-phosphoryl-ethanolamine are provided to media at a concentration range between about 10⁻⁶ to about 10⁻² M, more preferably at about 1×10⁻⁴ M.

Throughout the culture duration, the base medium is additionally supplemented with other components to induce synthesis or differentiation or to improve cell growth such as hydrocortisone, selenium, and L-glutamine.

Hydrocortisone has been shown in keratinocyte culture to promote keratinocyte phenotype and therefore enhance differentiated characteristics such as involucrin and keratinocyte transglutaminase content (Rubin et al., J. Cell Physiol., 138:208-214 (1986)). Therefore, hydrocortisone is a desirable additive in instances where these characteristics are beneficial such as in the formation of keratinocyte sheet grafts or skin constructs. Hydrocortisone may be provided at a concentration range of about 0.01 μg/ml to about 4.0 μg/ml, most preferably between about 0.4 μg/ml to 16 μg/ml.

Selenious acid is added to serum-free media to resupplement the trace elements of selenium normally provided by serum. Selenious acid may be provided at a concentration range of about 10⁻⁹ M to about 10⁻⁷ M; most preferably at about 5.3×10⁻⁸ M.

The amino acid L-glutamine is present in some nutrient bases and may be added in cases where there is none or insufficient amounts present. L-glutamine may also be provided in stable form such as that sold under the mark, GlutaMAX-1™ (Gibco BRL, Grand Island, N.Y.). GlutaMAX-1™ is the stable dipeptide form of L-alanyl-L-glutamine and may be used interchangeably with L-glutamine and is provided in equimolar concentrations as a substitute to L-glutamine. The dipeptide provides stability to L-glutamine from degradation over time in storage and during incubation that can lead to uncertainty in the effective concentration of L-glutamine in medium. Typically, the base medium is supplemented with preferably between about 1 mM to about 6 mM, more preferably between about 2 mM to about 5 mM, and most preferably 4 mM L-glutamine or GlutaMAX-1™.

Growth factors such as epidermal growth factor (EGF) may also be added to the medium to aid in the establishment of the cultures through cell scale-up and seeding. EGF in native form or recombinant form may be used. Human forms, native or recombinant, of EGF are preferred for use in the medium when fabricating a skin equivalent containing no non-human biological components. EGF is an optional component and may be provided at a concentration between about 1 to 15 ng/mL, more preferably between about 5 to 10 ng/mL.

Other supplements may also be added to the medium, such as one or more prostaglandins, transforming growth factors (including transforming growth factors alpha or beta), keratinocyte growth factor (KGF), connective tissue growth factor (CTGF), or mannose-6-phosphate (M6P), or a combination thereof.

Prostaglandin E₂ (PGE₂) is generated from the action of prostaglandin E synthases on prostaglandin H₂ (PGH₂). Several prostaglandin E synthases have been identified. To date, microsomal prostaglandin E synthase-1 emerges as a key enzyme in the formation of PGE₂. PGE₂ is supplemented to the medium preferably in the range from about 0.000038 μg/mL to about 0.760 μg/mL, more preferably from about 0.00038 μg/mL to about 0.076 μg/mL, most preferably from about 0.0038 μg/mL to about 0.038 μg/mL. The 16,16 PGE₂ form may also be supplemented in these ranges.

Transforming growth factor alpha (TFG-α) is produced in macrophages, brain cells, and keratinocytes, and induces epithelial development. It is closely related to EGF, and can also bind to the EGF receptor with similar effects. Preferably the long chain form of TFG-α is employed in the invention. TGF-alpha is a small (˜50 residue) protein that shares 30% structural homology with EGF and competes for the same surface-bound receptor site. It has been implicated in wound healing and promotes phenotypic changes in certain cells. TGF alpha or long-chain TGF alpha is supplemented to the medium preferably in the range from about 0.0005 μg/mL to about 0.30 μg/mL, more preferably from about 0.0050 μg/mL to about 0.03 μg/mL, most preferably from about 0.01 μg/mL to about 0.02 μg/mL. In some embodiments, the amount of supplemented TGF alpha is 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 120 ng/mL, 130 ng/mL, 140 ng/mL, 150 ng/mL, 160 ng/mL, 170 ng/mL, 180 ng/mL, 190 ng/mL, 200 ng/mL or more.

Supplementation of the base medium with keratinocyte growth factor 5 μg/mL may be used to support epidermalization. Keratinocyte growth factor (KGF) is supplemented to the medium preferably in the range from about 0.001 μg/mL to about 0.150 μg/mL, more preferably from about 0.0025 μg/mL to about 0.100 μg/mL, most preferably from about 0.005 μg/mL to about 0.015 μg/mL.

Supplementation of the base medium with mannose-6-phosphate (M6P) may be used to support epidermalization Mannose-6-Phosphate is supplemented to the medium preferably in the range from about 0.0005 mg/mL to about 0.0500 mg/mL.

CTGF (connective tissue growth factor) is a cysteine-rich, matrix-associated, heparin-binding protein. In vitro, CTGF mirrors some of the effects of TGF beta on skin fibroblasts, such as stimulation of extracellular matrix production, chemotaxis, proliferation and integrin expression. CTGF can promote endothelial cell growth, migration, adhesion and survival and is thus implicated in endothelial cell function and angiogenesis. CTGF binds to perlecan, a proteoglycan which has been localised in synovium, cartilage and numerous other tissues. CTGF has been implicated in extracellular matrix remodeling in wound healing, scleroderma and other fibrotic processes, as it is capable of upregulating both matrix metalloproteinases (MMPs) and their inhibitors (TIMPs). Therefore, CTGF has the potential to activate both the synthesis and degradation of the extracellular matrix.

The medium described above is typically prepared as set forth below. However, it should be understood that the components of the present invention may be prepared and assembled using conventional methodology compatible with their physical properties. It is well known in the art to substitute certain components with an appropriate analogous or functionally equivalent acting agent for the purposes of availability or economy and arrive at a similar result. Naturally occurring growth factors may be substituted with recombinant or synthetic growth factors that have similar qualities and results when used in the performance of the invention.

Media in accordance with the present invention are sterile. Sterile components are bought sterile or rendered sterile by conventional procedures, such as filtration, after preparation. Proper aseptic procedures were used throughout the following Examples. DMEM and F-12 are first combined and the individual components are then added to complete the medium. Stock solutions of all components can be stored at −20° C., with the exception of nutrient source that can be stored at 4° C. All stock solutions are prepared at 500× final concentrations listed above. A stock solution of insulin, transferrin and triiodothyronine (all from Sigma) is prepared as follows: triiodothyronine is initially dissolved in absolute ethanol in 1N hydrochloric acid (HCl) at a 2:1 ratio. Insulin is dissolved in dilute HCl (approximately 0.1N) and transferrin is dissolved in water. The three are then mixed and diluted in water to a 500× concentration. Ethanolamine and o-phosphoryl-ethanolamine are dissolved in water to 500× concentration and are filter sterilized. Progesterone is dissolved in absolute ethanol and diluted with water. Hydrocortisone is dissolved in absolute ethanol and diluted in phosphate buffered saline (PBS). Selenium is dissolved in water to 500× concentration and filter sterilized. EGF is purchased sterile and is dissolved in PBS. Adenine is difficult to dissolve but may be dissolved by any number of methods known to those skilled in the art. Serum albumin may be added to certain components in order to stabilize them in solution and are presently derived from either human or animal sources. For example, human serum albumin (HSA) or bovine serum albumin (BSA) may be added for prolonged storage to maintain the activity of the progesterone and EGF stock solutions. Recombinant forms of albumin have been developed, such as a human recombinant albumin, and their substitution instead of human and bovine serum-derived forms is preferred. The medium can be either used immediately after preparation or, stored at 4° C. If stored, EGF should not be added until the time of use.

In order to form the cell-matrix layer by the culture of matrix-producing cells, the medium is supplemented with additional agents that promote matrix synthesis and deposition by the cells. These supplemental agents are cell-compatible, defined to a high degree of purity and are free of contaminants. The medium used to produce the cell-matrix layer is termed “matrix production medium”.

To prepare the matrix production medium, the base medium is supplemented with an ascorbate derivative such as sodium ascorbate, ascorbic acid, or one of its more chemically stable derivatives such as L-ascorbic acid phosphate magnesium salt n-hydrate. Ascorbate is added to promote hydroxylation of proline and secretion of procollagen, a soluble precursor to deposited collagen molecules. Ascorbate has also been shown to be an important cofactor for post-translational processing of other enzymes as well as an upregulator of type I and type III collagen synthesis.

While not wishing to be bound by theory, supplementing the medium with amino acids involved in protein synthesis conserves cellular energy by not requiring the cells produce the amino acids themselves. The addition of proline and glycine is preferred as they, as well as the hydroxylated form of proline, hydroxyproline, are basic amino acids that make up the structure of collagen.

While not required, the matrix-production medium is optionally supplemented with a neutral polymer. The cell-matrix constructs of the invention may be produced without a neutral polymer, but again not wishing to be bound by theory, its presence in the matrix production medium may collagen processing and deposition more consistently between samples. One preferred neutral polymer is polyethylene glycol (PEG), which has been shown to promote in vitro processing of the soluble precursor procollagen produced by the cultured cells to matrix deposited collagen. Tissue culture grade PEG within the range between about 1000 to about 4000 MW (molecular weight), more preferably between about 3400 to about 3700 MW is preferred in the media of the invention. Preferred PEG concentrations are for use in the method may be at concentrations at about 5% w/v or less, preferably about 0.01% w/v to about 0.5% w/v, more preferably between about 0.025% w/v to about 0.2% w/v, most preferably about 0.05% w/v. Other culture grade neutral polymers such dextran, preferably dextran T-40, or polyvinylpyrrolidone (PVP), preferably in the range of 30,000-40,000 MW, may also be used at concentrations at about 5% w/v or less, preferably between about 0.01% w/v to about 0.5% w/v, more preferably between about 0.025% w/v to about 0.2% w/v, most preferably about 0.05% w/v. Other cell culture grade and cell-compatible agents that enhance collagen processing and deposition may be ascertained by the skilled routineer in the art of mammalian cell culture.

When the cell producing cells are confluent, and the culture medium is supplemented with components that assist in matrix synthesis, secretion, or organization, the cells are said to be stimulated to form a tissue-construct comprised of cells and matrix synthesized by those cells.

Therefore, a preferred matrix production medium formulation comprises: a base comprising Dulbecco's Modified Eagle's Medium (DMEM) (high glucose formulation, without L-glutamine) supplemented with either 4 mM L-glutamine or equivalent, 5 ng/ml epidermal growth factor, 0.4 μg/ml hydrocortisone, 1×10⁻⁴ M ethanolamine, 1×10⁻⁴ M o-phosphoryl-ethanolamine, 5 μg/ml insulin, 5 μg/ml transferrin, 20 ρM triiodothyronine, 6.78 ng/ml selenium, 50 ng/ml L-ascorbic acid, 0.2 μg/ml L-proline, and 0.1 μg/ml glycine. To the production medium, other pharmacological agents may be added to the culture to alter the nature, amount, or type of the extracellular matrix secreted. These agents may include polypeptide growth factors, transcription factors or inorganic salts to up-regulate collagen transcription. Examples of polypeptide growth factors include transforming growth factor-beta 1 (TGF-β1) and tissue-plasminogen activator (TPA), both of which are known to upregulate collagen synthesis. Raghow et al., Journal of Clinical Investigation, 79:1285-1288 (1987); Pardes et al., Journal of Investigative Dermatology, 100:549 (1993). An example of an inorganic salt that stimulates collagen production is cerium. Shivakumar et al., Journal of Molecular and Cellular Cardiology 24:775-780 (1992).

### II. Cell Types

An extracellular matrix-producing cell type for use in the invention may be any cell type capable of producing and secreting extracellular matrix components and organizing the extracellular matrix components to form a cell-matrix construct. More than one extracellular matrix-producing cell type may be cultured to form a cell-matrix construct. Cells of different cell types or tissue origins may be cultured together as a mixture to produce complementary components and structures similar to those found in native tissues. For example, the extracellular matrix-producing cell type may have other cell types mixed with it to produce an amount of extracellular matrix that is not normally produced by the first cell type. Alternatively, the extracellular matrix-producing cell type may also be mixed with other cell types that form specialized tissue structures in the tissue but do not substantially contribute to the overall formation of the matrix aspect of the cell-matrix construct, such as in certain skin constructs of the invention.

While any extracellular matrix-producing cell type may be used in accordance with this invention, the preferred cell types for use in this invention are derived from mesenchyme. More preferred cell types are fibroblasts, stromal cells, and other supporting connective tissue cells, most preferably human dermal fibroblasts found in human dermis for the production of a human dermal construct. Fibroblast cells, generally, produce a number of extracellular matrix proteins, primarily collagen. There are several types of collagens produced by fibroblasts, however, type I collagen is the most prevalent in vivo. Human fibroblast cell strains can be derived from a number of sources, including, but not limited to neonate male foreskin, dermis, tendon, lung, umbilical cords, cartilage, urethra, corneal stroma, oral mucosa, and intestine. The human cells may include but need not be limited to fibroblasts, but may include: smooth muscle cells, chondrocytes and other connective tissue cells of mesenchymal origin. It is preferred, but not required, that the origin of the matrix-producing cell used in the production of a tissue construct be derived from a tissue type that it is to resemble or mimic after employing the culturing methods of the invention. Dermal fibroblasts such as those derived from neonatal fibroblasts have wide application for most tissues in the body. Benefits of neonatal dermal fibroblasts is that they are believed to have plastic qualities, meaning that they are capable of transdifferentiation; are ideal for an hypoxic environment; and, are believed to be safe, biocompatible, and immuno-privileged as to not induce rejection by the subject. In another preferred embodiment, fibroblasts isolated by microdissection from the dermal papilla of hair follicles can be used to produce the matrix alone or in association with other fibroblasts. In the embodiment where a corneal-construct is produced, the matrix-producing cell is derived from corneal stroma. Cell donors may vary in development and age. Cells may be derived from donor tissues of embryos, neonates, or older individuals including adults. Embryonic progenitor cells such as mesenchymal stem cells may be used in the invention and induced to differentiate to develop into the desired tissue. Mesenchymal stem cells (MSCs; alternatively known as mesenchymal progenitor cells) are cells capable of expanding in culture and differentiating into mesenchymal tissue cells, including bone, cartilage, tendon, ligament, muscle, adipose, and marrow stroma. MSCs inefficiently synthesize, secrete, and/or organize extracellular matrix components (i.e., endogenous extracellular matrix production) under normal culture conditions. However, under culture conditions described further herein, they can contain themselves within an efficiently secreted extracellular matrix without exogenous matrix components (i.e., matrix components not produced by the cultured cells but introduced by other means).

MSCs can be obtained from a number of sources including, but not limited to, bone marrow, umbilical cord, placenta, amnion and other connective tissues (e.g. muscle, adipose, bone, tendon and cartilage). For example, umbilical cord MSC's can be isolated from umbilical cord blood, umbilical vein subendothelium, and the Wharton's Jelly. MCSs can further be isolated from three regions: the perivascular zone (umbilical cord perivascular cells or UCPVCs), the intervascular zone, placenta, amnion, and the subamnion (Troyer and Weiss, 2007). Alternatively, bone marrow-derived MSC's can be harvested from bone marrow and comprise non-hematopoietic, multipotential cells, support hematopoietic stem cell expansion, and can differentiate into various connective tissues.

Human cells, as well as those from other mammalian species including, but not limited to, equine, canine, porcine, bovine, ovine, or rodent (e.g., mouse or rat) can be used. The cells can be derived as primary cells from relevant tissues or more preferably from serially passaged or subcultured from established cell stocks or banks that have been screened against viral and bacterial contamination and tested for purity. In addition, cells that are spontaneously, chemically or virally transfected or recombinant cells or genetically engineered cells can also be used in this invention. Also, the cells can be recombinant or genetically-engineered. For example, the cells can be engineered to produce and deliver recombinant cell products such as growth factors, hormones, peptides or proteins, to a subject for a continuous amount of time or as needed when biologically, chemically, or thermally signaled due to the conditions present in the subject. Either long- or short-term gene product expression can be engineered. Long term expression is desirable when the cultured tissue construct is implanted or applied to a subject to deliver therapeutic products to the subject for an extended period of time. Conversely, short term expression is desired in instances where once a wound has healed, the gene products from the cultured tissue construct are no longer needed or can no longer be desired at the site. Cells can also be genetically engineered to express proteins or different types of extracellular matrix components which are either ‘normal’ but expressed at high levels or modified in some way to make a bioengineered complex comprising extracellular matrix and living cells that is therapeutically advantageous for improved wound healing, facilitated or directed neovascularization, or minimized scar or keloid formation.

Although human cells are preferred for use in the invention, the cells to be used in the method of the invention are not limited to cells from human sources. Cells from other mammalian species including, but not limited to, equine, canine, porcine, bovine, and ovine sources; or rodent species such as mouse or rat may be used. In addition, cells that are spontaneously, chemically or virally transfected or recombinant cells or genetically engineered cells may also be used in this invention. For those embodiments that incorporate more than one cell type, chimeric mixtures of normal cells from two or more sources, such as a chimeric mixture of autologous and allogeneic cells; mixtures of normal and genetically modified or transfected cells; mixtures of cells derived from different tissue or organ types; or, mixtures of cells of two or more species or tissue sources may be used.

Recombinant or genetically-engineered cells may be used in the production of the cell-matrix construct to create a tissue construct that acts as a drug delivery graft for a subject needing increased levels of natural cell products or treatment with a therapeutic. The cells may produce and deliver to the subject via the graft recombinant cell products, growth factors, hormones, peptides or proteins for a continuous amount of time or as needed when biologically, chemically, or thermally signaled due to the conditions present in the subject. Either long or short-term gene product expression is desirable, depending on the use indication of the cultured tissue construct. Long term expression is desirable when the cultured tissue construct is implanted to deliver therapeutic products to a subject for an extended period of time. Conversely, short term expression is desired in instances where the cultured tissue construct is grafted to a subject having a wound where the cells of the cultured tissue construct are to promote normal or near-normal healing or to reduce scarification of the wound site. Once the wound has healed, the gene products from the cultured tissue construct are no longer needed or may no longer be desired at the site. Cells may also be genetically engineered to express proteins or different types of extracellular matrix components which are either ‘normal’ but expressed at high levels or modified in some way to make a graft device comprising extracellular matrix and living cells that is therapeutically advantageous for improved wound healing, facilitated or directed neovascularization, or minimized scar or keloid formation. These procedures are generally known in the art, and are described in Sambrook et al, Molecular Cloning, A Laboratory Manual, Cold Spring Harbor Press, Cold Spring Harbor, N.Y. (1989), incorporated herein by reference. All of the above-mentioned types of cells are included within the definition of a “matrix-producing cell” as used in this invention.

The predominant major extracellular matrix component produced by fibroblasts is fibrillar collagen, particularly collagen type I. Fibrillar collagen is a key component in the cell-matrix structure; however, this invention is not to be limited to matrices comprised of only this protein or protein type. For instance, other collagens, both fibrillar and non-fibrillar collagen from the collagen family such as collagen types II, III, IV, V, VI, VII, VIII, IX, X, XI, XII, XIII, XIV, XV, XVI, XVII, XVIII, XIX, and others as they may be identified may be produced by use of the appropriate cell type. Similarly, other matrix proteins which can be produced and deposited using the current method include, but are not limited to elastin; proteoglycans such as decorin or biglycan; or glycoproteins such as tenascin; vitronectin; fibronectin; laminin, thrombospondin I, and glycosaminoglycans (GAG) such as hyaluronic acid (HA).

Fibroblast-derived bioengineered constructs can further comprise additional cell types capable of synthesizing, secreting, and organizing extracellular matrix to enhance extracellular matrix thicknesses. Such cell types can be MSCs, stromal cells, smooth muscle cells, chondrocytes and other connective tissue cells of mesenchymal origin.

The at least one additional cell type can be added in layered or admixed form. For layered bioengineered constructs, a first cell type is seeded upon a cell culture substrate and a second cell type is subsequently seeded atop the first layer of cells. Admixed constructs can be generated by varying the initial seeding ratios of the at least two cells types based at least in part upon desired construct attributes for therapeutic effect. For example, MSCs can be the first cell type and comprise 5%, 10%, 15%, 20%, 25%, 30%, 35%, 40%, 45%, 50%, 55%, 60%, 65%, 70%, 75%, 80%, 85%, 90%, 95%, 99%, or more of the initial cell seeding mixture. Fibroblasts, such as neonatal fibroblasts, dermal fibroblasts, papillary fibroblasts, reticular fibroblasts, or a combination thereof, can be the second cell type and comprise the remaining initial cell seed mixture. The total cell population at initial seeding can be between 1.0×10⁵ to1.0×10⁶ per cm².

For bioengineered constructs produced by admixing, initial seeding densities can also be determined based on the number of cells at the time of seeding, where the total cell mass desired is known at the time of seeding according to: aX+bY=Z; wherein X=Y=Z and a+b=1, but b>0 and a<1. For example, the desired cell seeding density is Z and Z=2.1×10⁵ cells/cm² (approximately) and aX and bY represent the number of fibroblasts and mesenchymal progenitor cells, respectively, in the total number of cells per square centimeter of area to be seeded represented by Z. Thus, where fibroblasts and MSCs each comprise 50% of the total cells seeded, the equation would be expressed as: aX+bY=Z cells/cm² where (0.5)(2.1×10⁵ cells)+(0.5)(2.1×10⁵ cells)=2.1×10⁵ totalcells/cm². Solving this equation leads to determining the initial seeding density of both of the at least two cell types: 1.05×10⁵ fibroblasts+1.05×10⁵ mesenchymal progenitor cells=2.1×10⁵ total cells/cm². When this seeding equation is employed, the following can be used: a=0 and b=1; a=0.1 and b=0.9; a=0.2 and b=0.8; a=0.3 and b=0.7; a=0.5 and b=0.5; a=0.8 and b=0.2.

Alternatively, hybrid bioengineered constructs can be produced by fibroblasts and MSCs, wherein X is constant (i.e., the number of fibroblasts is kept constant) where total number of fibroblasts in the total cell mass is known at the time of seeding according to: aX+bY=Z; wherein X=Y, a=1, b>0 and b<1, and Z=the calculated seeding density of the total cell mass. For example, if X=2.1×10⁵ fibroblasts and 50% MSCs is desired at seeding, the equation would be expressed as: aX+bY=Z, where (1)(2.1×10⁵ cells)+(0.5)(2.1×10⁵ cells)=Z total cells/cm². Solving this equation leads to determining the initial seeding density of both of the at least two cell types: 2.1×10⁵ fibroblasts+1.05×10⁵ mesenchymal progenitor cells=3.15×10⁵ total cells/cm². When this seeding equation is employed, the following can be used: a=1 and b=2; a=1 and b=1; a=1 and b=0.9; a=1 and b=0.8; a=1 and b=0.7; a=1 and b=0.5; a=1 and b=0.2.

As the aforementioned cell types may be used to produce the cell-matrix of the invention, they may also be delivered by the cell-matrix compositions of the invention where one or more cell-matrix sheets, in living, devitalized, or decellularized form are fabricated into a cell delivery device. These cell types may be delivered in contact with the cell-matrix of the invention to a site in a subject needing functional cells or cell products. As the cell-matrix compositions of the invention comprise collagen and collagen is a natural substrate for cell adhesion, these cells will naturally adhere to the cell-matrix composition. As the cell-matrix composition is also handleable, it allows for delivery of the cells and acts as a means for keeping the cells local to the delivery site.

### III. Culture Conditions and Methods

The system for the production of the cell-matrix layer may be either static or may employ a perfusion means to the culture media to exert a mechanical force against the forming cell-matrix layer to mimic in vivo forces. The application of appropriate stimuli may result in desirable properties, e.g., increased strength, as compared to static cultures. In the static system, the culture medium is still and relatively motionless as contrasted to the perfusion system where the medium is in motion. The perfusion of medium affects the viability of the cells and augments the development of the matrix layer. Perfusion means include, but are not limited to: using a magnetic stirbar or motorized impeller in the culture dish subjacent (below) or adjacent to the substrate carrier containing the culture membrane to stir the medium; pumping medium within or through the culture dish or chamber; gently agitating the culture dish on a shaking or rotating platform; or rolling, if produced in a roller bottle. Other perfusion means can be determined by one skilled in the art for use in the method of the invention. Other mechanical forces may be exerted by pulsing, flexing, undulating or stretching of the porous membrane during culture.

The cultures are maintained in an incubator to ensure sufficient environmental conditions of controlled temperature, humidity, and gas mixture for the culture of cells. Preferred conditions are between about 34° C. to about 38° C., more preferably 37±1° C. with an atmosphere between about 5-10±1% CO₂ and a relative humidity (Rh) between about 80-90%. One of skill in the art may easily determine environmental conditions that may be inside our outside the aforementioned environmental conditions depending on the cells being cultured or the step of culture being performed. Cultures may be removed from these conditions to ambient room temperature, air, and humidity such as during feeding, the seeding of additional cells, or other treatment without detriment to the cultures or their ability to form a cell-matrix sheet.

In the preferred embodiment, the cell-matrix construct is a dermal construct formed of dermal fibroblasts and their secreted matrix. Preferably, human dermal fibroblasts are used, derived as primary cells from dermis or more preferably from serially passaged or subcultured from established cell stocks or banks that have been screened against viral and bacterial contamination and tested for purity. Cells are cultured under sufficient conditions in growth medium to cause them to proliferate to an appropriate number for seeding the cells to the culture substrate on which to form a cell-matrix construct. Alternatively, cells from frozen cell stocks may be seeded directly to the culture substrate.

Seeding at superconfluency (i.e., greater than 100% confluency) increases the rate of extracellular matrix formation by bypassing the cellular growth phase. Thus, cells can be directly seeded at superconfluence from 100% confluence up to about 900% confluence, including in the range of about 300% to about 600% confluence to immediately produce an extracellular matrix. Superconfluency can also be achieved according to cell seeding densities per culture surface area and can be, for example, 1×10⁵, 2×10⁵, 3×10⁵, 4×10⁵, 5×10⁵, 6×10⁵, 7×10⁵, 8×10⁵, 9×10⁵, 1×10⁶ or more cells per cm². For example, 75 mm diameter inserts can be used which have an approximate culture surface area of 44 cm². Seeding a superconfluent number of cells (e.g., 3×10⁶ cells) on such an insert results in an initial seeding density of about 6.8×10⁵ cells/cm². Approximately 7.5×10⁶ cells can be seeded onto a 10 cm×10 cm rectangular insert to produce an initial seeding density of about 7.5×10⁵ cells/cm².

Alternatively, cells can be seeded at sub-confluence to proliferate prior to stimulating them to produce and organize an extracellular matrix. Sub-confluent cell density can be achieved by seeding between about 1×10⁵ cells/cm² to about 6.8×10⁵ cells/cm², between about 3×10⁵ cells/cm² to about 6.8×10⁵ cells/cm², or about 6.8×10⁵ cells/cm² (cells per square centimeter area of the surface).

Once sufficient cell numbers have been obtained, cells are harvested and seeded onto a suitable culture surface and cultured under appropriate growth conditions to form a confluent sheet of cells. In the preferred embodiment, the cells are seeded on a porous membrane that is submerged to allow medium contact from both below the cell culture through the pores and above with contact above on the top surface of the cell culture. Preferably, cells are suspended in either base or growth media and are seeded on the cell culture surface at a density between about 1×10⁵ cells/cm² to about 6.8×10⁵ cells/cm², more preferably between about 3×10⁵ cells/cm² to about 6.8×10⁵ cells/cm^{2,} and most preferably at about 6.8×10⁵ cells/cm² (cells per square centimeter area of the surface). Cultures are cultured in growth medium to establish the culture and are cultured to between about 80% to 100% confluence at which time they are induced chemically by changing the medium to matrix production medium in order to upregulate the synthesis and secretion of extracellular matrix. In an alternate method, cells are seeded directly in matrix production medium at least 80% confluence to eliminate the need to change from the basic medium to the production medium but it is a method that requires higher seeding densities. Higher seeding densities achieve a level of super-confluence, meaning that the cells are seeded at over 100% confluence up to about 900% confluence, including in the range of about 300% to about 600% confluence. When seeded at super-confluence, the growth phase of culturing cells on the membrane is bypassed and the cells are seeded in the matrix production medium in order to start matrix production at the time of seeding.

During the culture, fibroblasts secrete endogenous matrix molecules and organize the secreted matrix molecules to form a three dimensional tissue-like structure but do not exhibit significant contractile forces to cause the forming cell-matrix construct to contract and peel itself from the culture substrate. Media exchanges are made every two to three days with fresh matrix production medium and with time, the secreted matrix increases in thickness and organization. The time necessary for creating a cell-matrix construct is dependent on the initial seeding density, the cell type, the age of the cell line, and the ability of the cell line to synthesize and secrete matrix components. When fully formed, the cell-matrix constructs of the invention have bulk thickness due to the fibrous matrix produced and organized by the cells; they are not ordinarily confluent or overly confluent cell cultures where the cells may be loosely adherent to each other. The fibrous quality gives the constructs cohesive tissue-like properties unlike ordinary cultures because they resist physical damage, such as tearing or cracking, with routine handling in a clinical setting. In the fabrication of a cultured cell-matrix sheet from dermal fibroblasts to form a dermal construct, the cells will form an organized matrix around themselves on the cell culture surface preferably at least about 30 microns in thickness or more, more preferably between about 60 to about 120 microns thick across the surface of the membrane; however, thicknesses have been obtained in excess of 120 microns and are suitable for use in testing or clinical applications where such greater thicknesses are needed.

### VI. Culture Substrate

The matrix-producing cell is cultured in a vessel suitable for animal cell or tissue culture, such as a culture dish, flask, or roller-bottle, which allows for the formation of a three-dimensional tissue-like structure. Suitable cell growth surfaces on which the cells can be grown can be any biologically compatible material to which the cells can adhere and provide an anchoring means for the cell-matrix construct to form. Materials such as glass; stainless steel; polymers, including polycarbonate, polystyrene, polyvinyl chloride, polyvinylidene, polydimethylsiloxane, fluoropolymers, and fluorinated ethylene propylene; and silicon substrates, including fused silica, polysilicon, or silicon crystals may be used as a cell growth surfaces. The cell growth surface material may be chemically treated or modified, electrostatically charged, or coated with biologicals such as poly-l-lysine or peptides. An example of a peptide coating is RGD peptide.

While the tissue construct of the invention may be grown on a solid cell growth surface, a cell growth surface with pores that communicate both top and bottom surfaces of the membrane to allow bilateral contact of the medium to the developing tissue construct or for contact from only below the culture is preferred. Bilateral contact allows medium to contact both the top and bottom surfaces of the developing cell-matrix-based construct for maximal surface area exposure to the nutrients contained in the medium. Medium may also contact only the bottom of the forming cultured tissue construct so that the top surface may be exposed to air, as in the development of a cultured skin construct. The preferred culture vessel is one that utilizes a carrier or culture insert, a culture-treated permeable member such as a porous membrane that is suspended in the culture vessel containing medium. Typically, the membrane is secured to one end of a tubular member or framework that is inserted within and interfaces with a base, such as a petri or culture dish that can be covered with a lid. Culture vessels incorporating a carrier insert with a porous membrane are known in the art and are preferred for carrying out the invention and are described in a number United States patents in the field, some of which have been made commercially available, including for instance: U.S. Pat. Nos. 5,766,937, 5,466,602, 5,366,893, 5,358,871, 5,215,920, 5,026,649, 4,871,674, 4,608,342, the disclosures of which are incorporated herein. When these types of culture vessels are employed, the tissue-construct is produced on one surface of the membrane, preferably the top, upwardly facing surface and the culture is contacted by cell media on both top and bottom surfaces. The pores in the growth surface allow for the passage of culture media for providing nutrients to the underside of the culture through the membrane, thus allowing the cells to be fed bilaterally or solely from the bottom side. A preferred pore size is one that is small enough that it does not allow for the growth of cells through the membrane, yet large enough to allow for free passage of nutrients contained in culture medium to the bottom surface of the cell-matrix construct, such as by capillary action. Preferred pore sizes are about less than 7 microns but range between about 0.1 microns to about 7 microns, more preferably between about 0.2 microns to about 6 micron and most preferably about 0.4 micron to about 5 micron sized pores are employed. In the case of human dermal fibroblasts, the most preferred material is polycarbonate having a pore size is between about 0.4 to about 0.6 microns. The maximum pore size depends not only on the size of the cell but also the ability of the cell to alter its shape and pass through the membrane. It is important that the tissue-like construct adheres to the surface but does not incorporate or envelop the substrate so it is removable from it such as by peeling with minimal force. The size and shape of the tissue construct formed is dictated by the size of the vessel surface or membrane on which it grown. Substrates may be round, square, rectangular or angular or shaped with rounded corner angles, or irregularly shaped. Substrates may also be flat or contoured as a mold to produce a shaped construct to interface with a wound or mimic the physical structure of native tissue. To account for greater surface areas of the growth substrate, proportionally more cells are seeded to the surface and a greater volume of media is needed to sufficiently bathe and nourish the cells. When the cell-matrix-based tissue construct is finally formed, it is removed by peeling from the membrane substrate before grafting to a subject. It is to be appreciated that substrates can be pre-treated prior to cell seeding in order to improve bonding characteristics of the substrate by raising the surface energy. Pre-treatment can include, but is not limited to COOH and Long NH₂ treatment.

The cultured cell-matrix constructs of the invention do not rely on synthetic or bioresorbable members for, such as a mesh member for formation. A mesh member is organized as a woven, a knit, or a felt material. In systems where a mesh member is employed, the cells are cultured on the mesh member and grow on either side and within the interstices of the mesh to envelop and incorporate the mesh within the cultured tissue construct. The final construct formed by methods that incorporate such a mesh rely on it for physical support and for bulk. Examples of cultures tissue constructs that rely on synthetic mesh members are found in U.S. Pat. Nos. 5,580,781, 5,443,950, 5,266,480, 5,032,508, 4,963,489 to Naughton, et al.

The thickness of ECM can be optimized for a particular use in vivo. For example, thicker bioengineered constructs may be useful for sites in the body that experience physical agitation (e.g. knees) or for any application for which it is desired that the construct persist in vivo for an extended period of time.

The bulk thickness of the ECM confers cohesive tissue-like properties that are resistant to physical damage, such as tearing or cracking. Appropriate ECMs should have a thickness, which is at least about 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 220 μm, 240 μm, 260 μm, 280 μm, 300 μm, 320 μm, 340 μm, 360 μm, 380 μm, 400 μm, 450 μm, 500 μm, 550 μm, 600 μm, 650 μm, 700 μm, 750 μm, 800 μm, 850 μm, 900 μm, 950 μm, or more in thickness suitable for use in testing or clinical applications where such thicknesses are useful.

Seeding the cells on a porous membrane (i.e., culture insert) of a defined diameter can enhance bioengineered construct thicknesses by enhancing the rate at which extracellular matrices are produced, since it maximizes surface area exposure to the media's nutrients. The pores communicate through both top and bottom surfaces of the membrane to allow bilateral contact of the medium to the developing tissue construct or for contact from only below the culture. Media can also contact only the bottom of the forming cultured tissue construct, so that the top surface can be exposed to air, as in the development of a cultured skin construct. Typically, the membrane is secured to one end of a tubular member or framework that is inserted within and interfaces with a base, such as a petri or culture dish that can be covered with a lid. When these types of culture vessels are employed, the tissue-construct is produced on one surface of the membrane (e.g., the top, upwardly facing surface) and the culture is contacted by cell media on both top and bottom surfaces. The pore sizes are small enough that it does not allow for the growth of cells through the membrane, yet large enough to allow for free passage of nutrients contained in culture medium to the bottom surface of the bioengineered construct, such as by capillary action. For example, the pore sizes can be about less than 7 μm, between about 0.1 μm to about 7 μm, between about 0.2 μm to about 6 μm, or between about 0.4 μm to about 5 μm in diameter. The maximum pore size depends not only on the size of the cell but also the ability of the cell to alter its shape and pass through the membrane. It is important that the tissue-like construct adheres to the surface but does not incorporate or envelop the substrate so it is removable from it such as by peeling with minimal force. The size and shape of the tissue construct formed is dictated by the size of the vessel surface or membrane on which it is grown. Substrates can be round, square, rectangular or angular or shaped with rounded corner angles, or irregularly shaped. Substrates can also be flat or contoured as a mold to produce a shaped construct to interface with a wound or mimic the physical structure of native tissue. To account for greater surface areas of the growth substrate, proportionally more cells are seeded to the surface and a greater volume of media is needed to sufficiently bathe and nourish the cells. When the bioengineered-based tissue construct is finally formed, it is removed by peeling from the membrane substrate.

Perfusing the culture substrate to exert a mechanical force against the forming bioengineered layer to mimic in vivo forces can further enhance bioengineered construct thickness and strength. Perfusion means are well known in the art and include, but are not limited to, stirring the media using a magnetic stir bar or motorized impeller subjacent or adjacent to the substrate carrier containing the culture membrane; pumping media within or through the culture dish or chamber; gently agitating the culture dish on a shaking or rotating platform; or rolling if a roller culture bottle is used. Other mechanical forces can be exerted by pulsing, flexing, undulating or stretching of the porous membrane during culture.

During the culture, cells secrete endogenous matrix molecules and organize the secreted matrix molecules to form a three dimensional tissue-like structure but do not exhibit significant contractile forces to cause the forming bioengineered construct to contract and peel itself from the culture substrate. Suitable cell growth surfaces on which the cells can be grown can be any biologically compatible material to which the cells can adhere and provide an anchoring means for the bioengineered construct to form. Materials such as glass; stainless steel; polymers, including polycarbonate, poly(ether sulfones) (PES), polystyrene, polyvinyl chloride, polyvinylidene, polydimethylsiloxane, fluoropolymers, and fluorinated ethylene propylene; and silicon substrates, including fused silica, polysilicon, or silicon crystals can be used as a cell growth surface. The cell growth surface material can be chemically treated or modified, electrostatically charged, or coated with biologicals such as poly-l-lysine or peptides. An example of a chemical treatment that results in an electrostatically charged surface COOH and Long NH₂. An example of a peptide coating is RGD peptide. The cell growth surface can be treated with a synthetic or human form of extracellular matrix that assists with the attachment of the matrix producing cells so that the cells have a natural interface with the cell growth surface for attachment, orientation, and biochemical cues. When a synthetic or human form of extracellular matrix is used in this aspect, it is temporary because it is replaced by the cells over time in culture. The synthetic or human form of extracellular matrix, when deposited on the cell-growth surface range from matrix molecules dispersed across the surface, to molecular thickness, or to continuous thin film of between nanometer to micrometer thickness.

Fibronectin in natural and synthetic forms can be used to provide a coating to the culture substrate. The fibronectin forms which can be used, include but are not limited to: human fibronectin, human plasma-derived fibronectin, recombinant fibronectin, or synthetic forms such as ProNectin, which is a repeated peptide sequence derived and synthesized from a portion of natural human fibronectin. Coatings of natural, cell-culture produced or recombinant collagen can be provided to the substrate.

The cultured bioengineered constructs do not rely on synthetic or bioresorbable members, such as a mesh member, for formation and integrity; however, such members can be used. A mesh member can be a woven, knit, or felt like material. In systems where a mesh member is used, the cells are cultured on the mesh member and grow on either side and within the interstices of the mesh to envelop and incorporate the mesh within the cultured tissue construct. The final construct formed by methods that incorporate such a mesh rely on it for physical support and for bulk.

Certain constructs may be porous in structure. Porosity may be measured by the surface area attributed to pores in a histology image relative to the total surface area of the image. Certain constructs can have a porosity of at least 40%, 45%, 50%, 55%, 60%, 65%, 70%, 75%, 80%, 85%, 90% or more.

The average pore size within the extracellular matrix of bioengineered constructs can be engineered to form porous extracellular matrix and/or regulate pore size. Combined with a type and/or degree of crosslinking, defined average pore sizes can be chosen and controlled to yield constructs that have different rates of in vivo persistence and/or cell infiltration, ranging from “rapidly bioremodelable” to “moderately bioremodelable” to “prolonged bioremodelable” bioengineered constructs for tailored applicability to therapeutic uses. In addition, smaller pore sizes can be engineered to enhance barrier functions where prevention or inhibition of cell infiltration, such as undesirable host cell types, is useful.

Average pore size (diameter) can be engineered by varying the final temperature at which lyophilization, also known as freeze-drying, occurs. In this process, the bioengineered constructs are frozen such that the aqueous aspects of the bioengineered construct achieve a frozen state, after which, the bioengineered construct is subjected to a vacuum to remove the frozen water (ice) from the construct. Lyophilization creates and opens up the pore structure by removing the ice crystals that form in the matrix and the freezing temperature determines the resulting average pore size. Thus, performing lyophilization at colder freezing temperatures generates smaller pore sizes, whereas performing lyophilization at warmer freezing temperatures generates larger pore sizes. Thus, in one embodiment, the temperature can range between −100° C. and 0° C. with an average pore size of less than 5 to 10, 15, 20, 25, 30, 35, 40, 45, 50, 55, 60, 65, 70, 75, 80, 85, 90, 95, 100 or more microns (um) in size as the freezing temperature warms. In one embodiment, average pore size of less than 5, 10, 15, 20, 25, or 30 um in size or any range in between can be produced at a freezing temperature of −40° C. In another embodiment, average pore sizes of at least 30, 35, 40, 45, 50, 55, 60, 65, 70, 75, 80, 85, 90, 95, 100, or more um in size or any range in between can be produced at a freezing temperature of −10° C. Decreasing the rate toward reaching the freezing temperature can increase the uniformity of pore size. Thus, decreasing the rate to freezing by 10, 5, 4, 3, 2, 1, 0.9, 0.8, 0.7, 0.6, 0.5, 0.4, 0.3, 0.3, 0.1, or fewer ° C. per minute, or any range in between, can increase the uniformity of the pores in the construct.

Silk scaffolds can provide structural support, while illiciting minimal or no host immune response. The porosity of the porous silk fibroin scaffold can range from between about 10 microns to about 150 microns, 30 microns to about 45 microns, 50 microns to 100 microns, or 80 microns to 15-microns in diameter.

Average pore diameter of the silk scaffolds can be controlled by varying the solvent percentage. Silk fibers can be mixed with an organic solvent, such as ethanol or DMSO. By increasing the amount of organic solvent, the pore size of the silk scaffolds can be selectively decreased based upon a desired level of porosity. For example, dissolving 4% silk to 1% ethanol results in a silk scaffold that has an average pore diameter of 50-100 microns. A pore size between 50 and 100 microns is desirable for enhanced fibroblast infiltration and for allowing faster vascularization of the construct in vivo. A larger silk scaffold average pore diameter (e.g., about 80-150 microns) can be achieved by dissolving 3% silk into 0.5% ethanol. A silk scaffold with an average pore diameter of about 80-150 microns is desirable for more severe burn wounds because the larger pores allows for wound exudates to be cleared from the wound bed.

Silk fibroin can be derived from either natural or recombinant sources. A preferred natural source of silk fibroin is derived from the degummed silk fiber of a Bombyx Mori silkworm cocoon. A solution of silk fibroin is admixed with a water-miscible organic solvent such as an alcohol selected from the group consisting of ethyl alcohol, methyl alcohol, isopropyl alcohol, propanol, butanol; or dimethylsulfoxide (DMSO) or acetone. The silk fibroin solution is then cast or poured into a mold or directly into a culture insert that incorporates a porous/permeable culture membrane that provides for bilateral contact of culture medium both above and below the planar surface of the membrane and porous silk fibroin scaffold. The solution is then frozen for a time, then thawed and rinsed to remove solvent residues. The porous silk fibroin scaffolds are then autoclaved, gamma irradiated or e-beam sterilized to produce a sterile porous silk fibroin scaffold. After sterilization, the porous silk fibroin scaffold can be used as a culture substrate for cultured cells using the methods employed herein. After culturing cells on the porous silk fibroin scaffolds, the cells can also be devitalized using the methods employed herein. Other features can be added to the porous silk fibroin scaffold constructs, such as a silicone layer.

Silk scaffolds can be conditioned with substances useful for enhancing wound healing. For example, wet or dry silk scaffolds can be incubated with a solution containing one or more proteins for 5-10 minutes such that the final amount of protein adsorbed is in the range of 1 microgram to 1 milligram. Silk scaffolds and bioengineered constructs comprising silk scaffolds that are partially lyophilized (e.g., freeze dried for 3 hours at 0° C.) and frozen at −20° C. prior to incubation with protein solutions appear to maximize the amount of adsorbed protein. Autoclaving the silk scaffold prior to use in cell culture also appears to enhance in vivo degradation and thus reduced persistence.

The thickness of a bioengineered construct can be enhanced by releasing it from the culture substrate, so that it is allowed to contract without restraint. Such “controlled contraction” or “unrestrained contraction” can be monitored in real time and can be stopped after a desired amount of contraction and thickness has occurred. The living cells in the bioengineered construct exert contractile forces on the endogenous extracellular matrix that are mitigated by adherence of the bioengineered construct to the culture substrate. In the unrestrained contraction step, these contractile forces imparted by the cells are leveraged to increase the overall physical strength and thickness of the construct as compared to similarly prepared constructs that have not been subjected to unrestrained contraction after culturing. Controlled contraction can be induced by releasing the bioengineered construct from the culture substrate such as by using physical means such as by peeling or lifting it from the substrate, shaking it from the substrate, or by flexing the substrate. Release of the bioengineered construct can also be achieved by changing the temperature of the culture, especially when a thermoresponsive substrate is employed, or by using chemical means.

Controlled contraction is measured by time, by increased thickness, and by a decrease in surface area, as measured by the decrease in diameter or decrease of the width and length, of the construct. Contraction of the matrix by the cells appears to organize the fibers of the endogenous matrix such that they increase the overall strength of the matrix (e.g., suture retention strength), but not so much that the matrix becomes misshapen, distorted, wrinkled or loses an approximate planarity in its configuration. In other words, the flat planar aspect of the matrix is preserved, but the overall surface area decreases and thickness increases. When unrestrained contraction is measured by the overall increase of the bioengineered thickness, a percentage thickness increase or an actual increased thickness measure is used. When unrestrained contraction is measured by decrease in surface area, a percentage decrease in the surface area or an actual measure of decrease of one or more dimensions is used. Contraction can be measured by measuring the percentage decrease in the surface area of the tissue matrix, such as between 10%, 20%, 30%, 40%, 50%, 60%, 70%, 80% or more or any range in between. Contraction can be stopped, when appropriate, by devitalizing the cells such as described further herein.

### IV. Chemical Modifications

The cell-matrix constructs of the invention are either devitalized, to terminate the cells, or decellularized to remove the cells, depending upon their ultimate use in treating a subject.

The cell-matrix of the invention may be devitalized or decellularlized either on the membrane of the culture insert or it may first be removed from it. As the culture insert suspends the cell-matrix in the dish to allow bilateral contact with culture medium, the bilateral contact maybe leveraged when the cell-matrix is treated using a chemical devitalizing or decellularizing agent or when the cell-matrix construct is dried using air, light or irradiation. The culture insert is conveniently removable from the culture apparatus so it may be transferred to a different vessel where it may be subjected to or contacted with a devitalizing or decellularizing agent.

To devitalize cells in a cell-matrix means to terminate, but not remove, the cells, to form a non-living cell-matrix. The constructs of the invention may be devitalized, in other words, the matrix-producing cells that produce the endogenous extracellular matrix components to form the cell-matrix constructs are terminated. When the cells are terminated, they remain in the matrix they formed. Devitalizing agents and methods are preferably those that retain the cell-matrix integrity and structure.

One method for devitalizing the cells in the cell-matrix construct employs dehydrating or drying the construct to remove all or substantially call of the moisture in the construct. Means for removing moisture include dehydration in air, by freezing or by freeze-drying. To dehydrate the construct by air-drying, culture medium is removed from the vessel in which the cell-matrix construct is made and the cell-matrix construct is simply allowed to dehydrate for a sufficient time to allow the cells to die. Dehydration conditions vary in terms of temperature and relative humidity. Preferred dehydration temperatures range from above freezing temperature up to the denaturation temperature of the collagen (as measured by differential scanning calorimetry, or “DSC”) in the cell-matrix construct, for example, between about 0° C. to about 60° C. A more preferred dehydration temperature is ambient room temperature, about 18° C. to about 22° C. Relative humidity values that are lower, as in the range of about 0% to about 60%, are preferred; however, relative humidities comparative to room humidity, between about 10% Rh to about 40% Rh are also preferred. When dehydration is conducted by air-drying at ambient room temperature and humidity, the cell-matrix construct will have about 10% to about 40% w/w moisture, or less. Therefore, when air-drying the cell-matrix constructs of the invention, some level of moisture is retained. To freeze-dry the construct, also termed “lyophilization”, the cell-matrix is frozen and then placed in a vacuum environment to remove the moisture. Lyophilization techniques can be employed to the constructs disclosed in the present invention such that biological activity of multiple growth factors within the constructs remain uninterrupted. In one aspect, one-layer cell-matrix constructs can be taken straight out of culture and frozen at −80° C., and lyophilized overnight, such as between about 1 to about 15 hours, or longer. In another aspect, one-layer cell-matrix constructs can first be air-dried for about eight hours, and subsequently frozen at −80° C., and lyophilized overnight, such as between about 1 to about 15 hours, or longer.

After drying in ambient conditions or by freeze-drying, the cell-matrix is devitalized but still retains devitalized cells and cell remnants. Lyophilization can also impart qualities different than those that may result when dehydrating under ambient conditions. Such qualities, in one embodiment, exhibits a more porous and open fibrous matrix structure.

Chemical means may also be employed to devitalize the cells in the cell-matrix construct. Water to osmotically terminate the cells may be used. Cell-matrix constructs are immersed in sterile, pure water for a time sufficient to allow for hypotonic swelling to cause the cells to lyse. After the cells lyse, the cell-matrix is devitalized but still retains devitalized cells and cell remnants. When water is used, it may also be mixed with other substances such as peracetic acid or hydrogen peroxide, or salts, or a combination thereof. For example, a devitalizing solution of peracetic acid between about 0.05% to about 3% v/v in water may be used. This devitalizing agent may also be buffered or contain a high salt concentration to prevent excessive swelling of the cell-matrix when terminating the cells.

Organic solvents and organic solvent solutions may be used as devitalizing agents in the invention. Organic solvents are capable of displacing the water in a cell-matrix construct to terminate, therefore, devitalize the cells in the cell-matrix. Preferably, the organic solvent employed to remove water is one that leaves no residues when they it is removed from the construct. Preferred organic solvents include alcohols, such as ethyl alcohol, methyl alcohol and isopropyl alcohol; or acetone. For the purpose of illustration, cell-matrix constructs are immersed in sterile ethyl alcohol for a time sufficient to displace water in the cell-matrix construct and devitalize the cells. The cell-matrix constructs are then removed from the ethyl alcohol and then exposed to air for a time sufficient to allow the absorbed ethyl alcohol in the cell-matrix construct to evaporate. After evaporation of solvent, the cell-matrix is devitalized but still retains the devitalized cells and cell remnants and the cell-matrix is dehydrated.

Other means to devitalize the cells include subjecting the cell-matrix constructs to ultraviolet light or gamma irradiation. These means may be used in conjunction with hypotonic swelling of the cell-matrix construct with water, or other chemical devitalizing means or with air and freezing devitalizing means.

To decellularize a cell-matrix of the invention means to remove the cells from the cell-matrix such that cells, cell remnants are removed from the cell-matrix to result in a extracellular matrix without the cells that produced it. The cell-matrix constructs of the invention may be decellularlized, in other words, the matrix-producing cells that produce the endogenous extracellular matrix components to form a the cell-matrix constructs are removed from the cell-matrix. When the cells are removed, a cell-matrix endogenously produced by cultured cells now remains but without those cells that formed it. One preferred method for decellularizing the cell-matrix constructs of the invention uses a series of chemical treatments to remove the cells, cell remnants, and residual cellular DNA and RNA. Other non-collagenous and non-elastinous extracellular matrix components may also be removed or reduced with the agents and methods used to decellularized the cell-matrix constructs, such as glycoproteins, glycosaminoglycans, proteoglycans, lipids, and other non-collagenous proteins. The removal of cells and non-collagenous and non-elastinous components from the cell-matrix yields a cell-matrix that is acellular and comprised of all or substantially all collagen with some lesser amounts of elastin.

The cell-matrix construct is first treated by contacting it with an effective amount of chelating agent, preferably physiologically alkaline to controllably limit swelling of the cell-matrix. Chelating agents enhance removal of cells, cell debris and basement membrane structures from the matrix by reducing divalent cation concentration. Alkaline treatment dissociates glycoproteins and glycosaminoglycans from the collagenous tissue and saponifies lipids. Chelating agents known in the art which may be used include, but are not limited to, ethylenediaminetetraacetic acid (EDTA) and ethylenebis(oxyethylenitrilo)tetraacetic acid (EGTA). EDTA is a preferred chelating agent and may be made more alkaline by the addition of sodium hydroxide (NaOH), calcium hydroxide Ca(OH)₂, sodium carbonate or sodium peroxide. EDTA or EGTA concentration is preferably between about 1 to about 200 mM; more preferably between about 50 to about 150 mM; most preferably around about 100 mM. NaOH concentration is preferably between about 0.001 to about 1 M; more preferably between about 0.001 to about 0.10 M; most preferably about 0.01 M. Other alkaline or basic agents can be determined by one of skill in the art to bring the pH of the chelating solution within the effective basic pH range. The final pH of the basic chelating solution should be preferably between about 8 and about 12, but more preferably between about 11.1 to about 11.8. In the most preferred embodiment, the cell-matrix is contacted with a solution of 100 mM EDTA/10 mM NaOH in water. The cell-matrix is contacted preferably by immersion in the alkaline chelating agent while more effective treatment is obtained by gentle agitation of the construct and the solution together for a time for the treatment step to be effective.

The cell-matrix is then contacted with an effective amount of acidic solution, preferably containing a salt. Acid treatment also plays a role in the removal of glycoproteins and glycosaminoglycans as well as in the removal of non-collagenous proteins and nucleic acids such as DNA and RNA. Salt treatment controls swelling of the collagenous matrix during acid treatment and is involved with removal of some glycoproteins and proteoglycans from the collagenous matrix. Acid solutions known in the art may be used and may include but are not limited to hydrochloric acid (HCl), acetic acid (CH₃COOH) and sulfuric acid (H₂SO₄). A preferred acid is hydrochloric acid (HCl) at a concentration preferably between about 0.5 to about 2 M, more preferably between about 0.75 to about 1.25 M; most preferably around 1 M. The final pH of the acid/salt solution is preferably between about 0 to about 1, more preferably between about 0 and 0.75, and most preferably between about 0.1 to about 0.5. Hydrochloric acid and other strong acids are most effective for breaking up nucleic acid molecules while weaker acids are less effective. Salts that may be used are preferably inorganic salts and include but are not limited to chloride salts such as sodium chloride (NaCl), calcium chloride (CaCl₂), and potassium chloride (KCl) while other effective salts may be determined by one of skill in the art. Preferably chloride salts are used at a concentration preferably between about 0.1 to about 2 M; more preferably between about 0.75 to about 1.25 M; most preferably around 1 M. A preferred chloride salt for use in the method is sodium chloride (NaCl). In the most preferred embodiment, the cell-matrix is contacted with 1 M HCl/1 M NaCl in water. The cell-matrix is contacted preferably by immersion in the acid/salt solution while effective treatment is obtained by gentle agitation of the construct and the solution together for a time for the treatment step to be effective.

The cell-matrix is then contacted with an effective amount of salt solution which is preferably buffered to about a physiological pH. The buffered salt solution neutralizes the material while reducing swelling. Salts that may be used are preferably inorganic salts and include but are not limited to chloride salts such as sodium chloride (NaCl), calcium chloride (CaCl₂), and potassium chloride (KCl); and nitrogenous salts such as ammonium sulfate (NH₃SO₄) while other effective salts may be determined by one of skill in the art. Preferably chloride salts are used at a concentration preferably between about 0.1 to about 2 M; more preferably between about 0.75 to about 1.25 M; most preferably about 1 M. A preferred chloride salt for use in the method is sodium chloride (NaCl). Buffering agents are known in the art and include but are not limited to phosphate and borate solutions while others can be determined by the skilled artisan for use in the method. One preferred method to buffer the salt solution is to add phosphate buffered saline (PBS) preferably wherein the phosphate is at a concentration from about 0.001 to about 0.02 M and a salt concentration from about 0.07 to about 0.3 M to the salt solution. A preferred pH for the solution is between about 5 to about 9, more preferably between about 7 to about 8, most preferably between about 7.4 to about 7.6. In the most preferred embodiment, the tissue is contacted with 1 M sodium chloride (NaCl)/10 mM phosphate buffered saline (PBS) at a pH of between about 7.0 to about 7.6. The cell-matrix is contacted preferably by immersion in the buffered salt solution while effective treatment is obtained by gentle agitation of the tissue and the solution together for a time for the treatment step to be effective.

After chemical cleaning treatment, the cell-matrix is then preferably rinsed free of chemical cleaning agents by contacting it with an effective amount of rinse agent. Agents such as water, isotonic saline solutions and physiological pH buffered solutions can be used and are contacted with the cell-matrix for a time sufficient to remove the cleaning agents. A preferred rinse solution is physiological pH buffered saline such as phosphate buffered saline (PBS). Other means for rinsing the cell-matrix of chemical cleaning agents can be determined by one of skill in the art. The cleaning steps of contacting the cell-matrix with an alkaline chelating agent and contacting the cell-matrix with an acid solution containing salt may be performed in either order to achieve substantially the same cleaning effect. The solutions may not be combined and performed as a single step, however.

The result of decellularizing a cell-matrix construct is an endogenously produced collagenous matrix produced by cultured cells that has been decellularized of the cells that produced it. A further result of decellularized cell-matrix construct is an endogenously produced collagenous matrix produced by cultured cells that has been decellularized of the cells that produced it and has a removal or reduction of non-collagenous and non-elastinous extracellular matrix components.

In some embodiments, the cell-matrix constructs may be first devitalized to terminate the cells and then decellularlized to remove the devitalized cells.

The devitalized or decellularized cell-matrix constructs may be used in a current state but they may be further modified with chemical treatments, physical treatments, the addition of other substances such as drugs, growth factors, cultured cells, other matrix components of natural, biosynthetic, polymeric origin, and they may be combined with medical devices such as stents and closure devices for treating patent foramen ovale defects in the heart.

Crosslinking The decellurized or devitalized cell-matrix may be crosslinked using a crosslinking agent to control its rate of bioremodeling and to either increase its persistence when implanted or engrafted into a living body. It may be crosslinked and used as a single layer construct or it may be combined or manipulated to create different types of constructs. The crosslinking methods of the invention also provide for methods of bonding cell-matrix sheets, or portions thereof, together.

The cell-matrix is preferably a planar sheet structure that can be used to fabricate various types of cell-matrix constructs to be used as a prosthesis with the shape of the prosthesis ultimately depending on its intended use. To form prostheses of the invention, the devitalized or decellularized cell-matrix sheets should be fabricated using a method that preserves the bioremodelability of the matrix sheets but also is able to enhance its strength and structural characteristics for its performance as a replacement tissue. Flat-sheet constructs of the invention comprise either devitalized or decellularized cell-matrix sheets, or devitalized and decellularized matrix sheets (such as one devitalized cell-matrix sheet and one decellularized cell-matrix sheet) layered to contact another, and bonded together. Tubular constructs of the invention comprise either a devitalized or decellularized matrix sheet rolled over itself to at least a minimum degree to contact itself. The area of contact between matrix sheets or a matrix sheet to itself is a bonding region.

Multilayer crosslinked constructs. In a preferred embodiment, the prosthetic device of this invention has two or more superimposed matrix sheets that are bonded together to form a flat-sheet construct. As used herein, “bonded collagen layers” means composed of two or more cell-matrix sheets of the same or different origins or profiles treated in a manner such that the layers are superimposed on each other and are sufficiently held together by self-lamination and chemical bonding.

A preferred embodiment of the invention is directed to flat sheet prostheses, and methods for making and using flat sheet prostheses, comprising of two or more matrix sheets that are bonded and crosslinked. Due to the flat sheet structure of the matrix sheets, the prosthesis is easily fabricated to comprise any number of layers, preferably between 2 and 20 layers, more preferably between 2 and 10 layers, with the number of layers depending on the strength and bulk necessary for the final intended use of the construct. Alternatively, as the ultimate size of a superimposed arrangement is limited by the size of the matrix sheets, the layers may be staggered, in a collage arrangement to form a sheet construct with a surface area larger than the dimensions of any individual matrix sheet but without continuous layers across the area of the arrangement.

In the fabrication of a multilayer construct comprising matrix sheets, an aseptic environment and sterile tools are preferably employed to maintain sterility. To form a multilayer construct of matrix sheets, a first sterile rigid support member, such as a rigid sheet of polycarbonate, is laid down. If the matrix sheets are still not in a hydrated state from the devitalizing or decellularizing processes, they are hydrated in aqueous solution, such as water or phosphate buffered saline. Matrix sheets are blotted with sterile absorbent cloths to absorb excess water from the material. A first matrix sheet is laid on the polycarbonate sheet and is manually smoothed to the polycarbonate sheet to remove any air bubbles, folds, and creases. A second matrix sheet is laid on the top of the first sheet, again manually removing any air bubbles, folds, and creases. This layering is repeated until the desired number of layers for a specific application is obtained.

After layering the desired number of matrix sheets, they are then dehydrated together. While not wishing to be bound by theory, dehydration brings the extracellular matrix components, such as collagen fibers, in the layers together when water is removed from between the fibers of the adjacent matrix sheets. The layers may be dehydrated either open-faced on the first support member or, between the first support member and a second support member, such as a second sheet of polycarbonate, placed before drying over the top layer and fastened to the first support member to keep all the layers in flat planar arrangement together with or without compression. To facilitate dehydration, the support member may be porous to allow air and moisture to pass through to the dehydrating layers. The layers may be dried in air, in a vacuum, or by chemical means such as by acetone or an alcohol such as ethyl alcohol or isopropyl alcohol. Dehydration by air-drying may be done to room humidity, between about 0% Rh to about 60% Rh, or less; or about 10% to about 40% w/w moisture, or less. Dehydration may be easily performed by angling the superimposed matrix layers to face a sterile airflow of a laminar flow cabinet for at least about 1 hour up to 24 hours at ambient room temperature, approximately 20° C., and at room humidity. Dehydration conducted by vacuum or chemical means will dehydrate the layers to moisture levels lower than those achieved by air-drying.

In an optional step, the dehydrated layers are rehydrated or, alternatively, rehydrated and dehydrated again. As mentioned above, the dehydration brings the extracellular matrix components of adjacent matrix layers together and crosslinking those layers together forms chemical bonds between the components to bond the layers. To rehydrate the layers, they are peeled off the porous support member together and are rehydrated in an aqueous rehydration agent, preferably water, by transferring them to a container containing aqueous rehydration agent for at least about 10 to about 15 minutes at a temperature between about 4° C. to about 20° C. to rehydrate the layers without separating or delaminating them. The matrix layers are then crosslinked together by contacting the layered matrix sheets with a crosslinking agent, preferably a chemical crosslinking agent that preserves the bioremodelability of the matrix layers.

Crosslinking the bonded prosthetic device also provides strength and durability to the device to improve handling properties. Various types of crosslinking agents are known in the art and can be used such as carbodiimides, genipin, transglutaminase, ribose and other sugars, nordihydroguaiaretic acid (NDGA), oxidative agents, ultraviolet (UV) light and dehydrothermal (DHT) methods. Besides chemical crosslinking agents, the layers may be bonded together with biocompatible fibrin-based glues or medical grade adhesives such as polyurethane, vinyl acetate or polyepoxy. One preferred biocompatible adhesive is silk fibroin, that is a 4-8% silk fibroin solution disposed at the bonding region between adjacent layers of tissue matrix that is activated using methyl alcohol. Biocompatible glues or adhesives may be used to bond crosslinked or uncrosslinked layers, or both, together to form bioengineered constructs of the invention.

A preferred biocompatible adhesive is silk fibroin, that is about a 2-8% silk fibroin solution disposed at the bonding region between adjacent layers of tissue matrix. In one aspect, two or more cell-matrix constructs described above can be combined using biocompatible adhesive biomaterials. As an example, the silk fibroin solution can be obtained from Bombyx mori silkworm, which can be processed to obtain a sericin-free compound, which, in one aspect, can be used as a biocompatible, silk adhesive. Bombyx mori consists primarily of glycine and alanine repeats that dominate the structure. The fibroin chain consists of two basic polypeptide sequences, crystalline and less ordered polypeptides that alternate regularly. The basic sequence of the ‘crystalline’ polypeptides is of -(Ala-Gly)ₙ- that adopts a β-sheet structure, whereas the ‘less ordered’ polypeptides contain additional amino acids, in particular, tyrosine, valine and acidic as well as basic amino acids. It is to be appreciated that a silk fibroin derived from recombinant source may be used to achieve a similar biocompatible adhesive properties to carry out the invention.

Briefly, in one aspect, cocoons of B. mori silkworm are boiled for 20 to 30 minutes in an aqueous solution comprising 0.02 M Na₂CO₃. In order to extract the glue-like sericin proteins, the cocoons are subsequently rinsed. In one embodiment, the extracted silk fibroin is dissolved in 9.3 M Lithium-Bromide (LiBr) solution at about 60° C. for about 4 hours, which yields a 20% weight by volume (w/v) solution. The resulting solution is subsequently dialyzed against distilled water using a Slide-a-Lyzer dialysis cassette (MWCO 3,500, Pierce) at room temperature for 48 h to remove the salt, however any dialyzing procedure is within the contemplation of the invention. The resulting dialysate is centrifuged in duplicate, each at −20° C. for 20 minutes in order to remove impurities and aggregates formed during the dialysis step.

A preferred crosslinking agent is 1-ethyl-3-(3-dimethylaminopropyl)carbodiimide hydrochloride (EDC). In an another preferred method, sulfo-N-hydroxysuccinimide is added to the EDC crosslinking agent as described by Staros, J. V., Biochem. 21, 3950-3955, 1982. In the most preferred method, EDC is solubilized in water at a concentration preferably between about 0.1 mM to about 100 mM, more preferably between about 1.0 mM to about 10 mM, most preferably at about 1.0 mM. Besides water, phosphate buffered saline or (2-[N-morpholino]ethanesulfonic acid) (MES) buffer may be used to dissolve the EDC. Other agents may be added to the solution, such as acetone or an alcohol, up to 99% v/v in water, typically 50%, to make crosslinking more uniform and efficient. These agents remove water from the layers to bring the matrix fibers together to promote crosslinking between those fibers. The ratio of these agents to water in the crosslinking agent can be used to regulate crosslinking EDC crosslinking solution is prepared immediately before use as EDC will lose its activity over time. To contact the crosslinking agent to the matrix layers, the hydrated, bonded matrix layers are transferred to a container such as a shallow pan and the crosslinking agent gently decanted to the pan ensuring that the matrix layers are both covered and free-floating and that no air bubbles are present under or between the matrix layers. The container is covered and the matrix layers are allowed to crosslink for between about 4 to about 24 hours, more preferably between 8 to about 16 hours at a temperature between about 4° C. to about 20° C. Crosslinking can be regulated with temperature: At lower temperatures, crosslinking is more effective as the reaction is slowed; at higher temperatures, crosslinking is less effective as the EDC is less stable.

Alternatively, bioengineered constructs can be crosslinked using dehydrothermal (DHT) crosslinking methods that form covalent bonds between the adjacent carboxy and amino groups on the protein fibers via a condensation reaction when the implants are exposed to controlled heat while under a vacuum (typically 120° C. dry heat for up to 24 hrs). In this treatment, water molecules are driven off from the individual fibers often leading to complex changes in the molecular positioning of amino acids in the collagen chain and possible oxidative damage. DHT can be advantageous over chemical cross-linking for certain regenerative medicine applications since this process does not introduce potentially cytotoxic or inflammatory chemicals into the implants for therapeutic use which would stimulate the patient's immune responses.

DHT has potential to provide high strength to collagen matrixes (˜50 MPa), but it is known to partially denature the collagen fibers due to the molecular repositioning of the amino acids within the collagen fibers. The greater number of crosslinks made in a material will typically provide greater durability when the material is exposed to digestive enzymes. However, it is also known that certain protein enzymes only cleave at specific target sites which can not be exposed within triple helical domains of collagen fibers unless and until the protein has been denatured. The level of denaturation that occurs during cross-linking of collagen implantables can be minimized in order to avoid the possible rapid degradation of the matrices by non-specific proteases upon implantation into the patient. Levels of DHT crosslinking in collagenous matrices is typically measured by changes in shrinkage temperature, mechanical loading or sensitivity to enzymatic digestions (e.g., collagenase, trypsin, etc.) of the collagen fibers. The effects of drying and thermal treatment of collagen can also be observed using X-ray diffraction to observe the changes in axial packing of collagen molecules in fibers as dehydration occurs. Layered and/or crosslinked bioengineered constructs can be formed into a number of form factors, such as tubular constructs, based on well known techniques (see, for example, U.S. Pat. No. 5,712,163 to Parenteau, PCT Publication No. WO 95/31473, PCT Publication No. WO 00/29553, and PCT Publication No. WO 2009/070720).

After crosslinking, the crosslinking agent is decanted and disposed of and the crosslinked multi-layer matrix constructs are rinsed by contacting them with a rinse agent to remove residual crosslinking agent. A preferred rinse agent is water or other aqueous solution. Preferably, sufficient rinsing is achieved by contacting the crosslinked multi-layer matrix constructs three times with equal volumes of sterile water for about one minute and forty-five minutes for each rinse.

Tubular constructs. In another preferred embodiment, the matrix construct of this invention is a tubular construct formed from a single, generally rectangular matrix sheet. The matrix sheet is rolled so that one edge meets and overlaps an opposing edge. The overlap serves as a bonding region. The tubular construct formed from a matrix sheet may be fabricated in various diameters, lengths, and number of layers and may incorporate other components depending on the indication for its use.

To form a tubular construct, a mandrel is chosen with a diameter measurement that will determine the diameter of the formed construct. The mandrel is preferably cylindrical or oval in cross section and made of glass, stainless steel or of a nonreactive, medical grade composition. The mandrel may be straight, curved, angled, it may have branches or bifurcations, or a number of these qualities. The number of layers intended for the tubular construct to be formed corresponds with the number of times a matrix sheet is wrapped around a mandrel and over itself. The number of times the matrix sheet can be wrapped depends on the dimensions of the processed matrix sheet. For a two layer tubular construct, the width of the matrix sheet must be sufficient for wrapping the sheet around the mandrel at least twice. It is preferable that the width be sufficient to wrap the sheet around the mandrel the required number of times and an additional percentage more as an overlap, between about 5% to about 20% of the mandrel circumference, to secure the bonding region and to ensure a tight seam. Similarly, the length of the mandrel will dictate the length of the tube that can be formed on it. For ease in handling the construct on the mandrel, the mandrel should be longer than the length of the construct so the mandrel, and not the construct being formed, is contacted when handled.

It is preferred that the mandrel is provided with a covering of a nonreactive, medical grade quality, elastic, rubber or latex material in the form of a sleeve. While a tubular matrix sheet construct may be formed directly on the mandrel surface, the sleeve facilitates the removal of the formed tube from the mandrel and does not adhere to, react with, or leave residues on the matrix sheet. To remove the formed construct, the sleeve may be pulled from one end off the mandrel to carry the construct from the mandrel with it. Because the matrix sheet only lightly adheres to the sleeve and is more adherent to other matrix sheet, fabricating tubes from matrix sheets is facilitated as the tubulated contract may be removed from the mandrel without stretching or otherwise stressing or risking damage to the construct. In the most preferred embodiment, the sleeve comprises KRATON® (Shell Chemical Company), a thermoplastic rubber composed of styrene-ethylene/butylene-styrene copolymers with a very stable saturated midblock.

For simplicity in illustration, a two-layer tubular construct with a 4 mm diameter and a 10% overlap is formed on a mandrel having about a 4 mm diameter. The mandrel is provided with a KRATON® sleeve approximately as long as the length of the mandrel and longer than the construct to be formed on it. A matrix sheet is trimmed so that the width dimension is about 28 mm and the length dimension may vary depending on the desired length of the construct. In the sterile field of a laminar flow cabinet, the matrix sheet is then formed into a tube by the following process. The matrix sheet is moistened along one edge and is aligned with the sleeve-covered mandrel and, leveraging the adhesive nature of the matrix sheet, it is “flagged” along the length of the sleeve-covered mandrel and dried in position for at least 10 minutes or more. The flagged matrix sheet is then hydrated and wrapped around the mandrel and then over itself one full revolution plus 10% of the circumference, for a 110% overlap, to serve as a bonding region and to provide a tight seam.

For the formation of single layer tubular construct, the matrix sheet must be able to wrap around the mandrel one full revolution and at least about a 5% of an additional revolution as an overlap to provide a bonding region that is equal to about 5% of the circumference of the construct. For a two-layer construct, the matrix sheet must be able to wrap around the mandrel at least twice and preferably an additional 5% to 20% revolution as an overlap. While the two-layer wrap provides a bonding region of 100% between the matrix sheet surfaces, the additional percentage for overlap ensures a tight, impermeable seam. For a three-layer construct, the matrix sheet must be able to wrap around the mandrel at least three times. The construct may be prepared with any number of layers as limited by the dimensions of the matrix sheet and the specifications desired. Typically, a tubular construct will have 10 layers or less, such as between 2 to 6 layers or between 2 or 3 layers with varying degrees of overlap. After wrapping, any air bubbles, folds, and creases are smoothed out from under the material and between the layers.

Matrix sheets may be rolled onto the mandrel either manually or with the assistance of an apparatus that aids for even tensioning and smoothing out of air or water bubbles or creases that can occur under the mandrel or between the layers of the wrapped matrix sheet. The apparatus would have a surface that the mandrel can contact along its length as it is turned to wrap the matrix sheet.

The layers of the wrapped matrix sheet are then bonded together by employing the methods and agents used in bonding and crosslinking flat-sheet constructs made from matrix sheets. After crosslinking and rinsing, the wrapped dehydrated ICL constructs may be then pulled off the mandrel via the sleeve or left on for further processing. The constructs may be rehydrated in an aqueous solution, preferably water, by transferring them to a room temperature container containing rehydration agent for at least about 10 to about 15 minutes to rehydrate the layers without separating or delaminating them.

### Combination Products

Other materials may be added to ECMs to further enhance bioactivity or function when administered in vivo.

For example, antimicrobial agents, drugs, growth factors, cytokines, genetic material and cultured cells can be incorporated in or on the bioengineered constructs, layers therein, and/or scaffolds.

Where the bioengineered constructs contact with blood in their use, as in the circulatory system, they can be rendered non-thrombogenic by applying heparin to the construct, to all surfaces of the construct or one side only in a flat-sheet construct or either luminally or abluminally for a tubular construct. Heparin can be applied to the construct, by a variety of well-known techniques. For illustration, heparin can be applied to the construct in the following three ways. First, benzalkonium heparin (BA-Hep) isopropyl alcohol solution is applied to the prosthesis by vertically filling the lumen or dipping the prosthesis in the solution and then air-drying it. This procedure treats the collagen with an ionically bound BA-Hep complex. Second, EDC can be used to activate the heparin and then to covalently bond the heparin to the collagen fiber. Third, EDC can be used to activate the collagen, then covalently bond protamine to the collagen and then ionically bond heparin to the protamine.

Synthetic materials can be disposed upon at least one surface of the bioengineered constructs. The synthetic material can be in the form of a sheet, superimposed or staggered upon the bioengineered construct to form a synthetic layer on the bioengineered layer. One class of synthetic materials, preferably biologically compatible synthetic materials, comprises polymers. Such polymers include but are not limited to the following: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolid-es) (PLGA), polyanhydrides, and polyorthoesters or any other similar synthetic polymers that can be developed that are biologically compatible. The term “biologically compatible, synthetic polymers” also includes copolymers and blends, and any other combinations of the forgoing either together or with other polymers generally. The use of these polymers will depend on given applications and specifications required. For example, biologically compatible synthetic materials can also be biodegradable such that, when implanted into the body of a subject, biodegrade over time. When disposed on a bioengineered construct, the combination construct comprises a biodegradable layer and a bioremodelable layer. A more detailed discussion of these polymers and types of polymers is set forth in Brannon-Peppas, Lisa, “Polymers in Controlled Drug Delivery,” Medical Plastics and Biomaterials, November 1997, which is incorporated by reference as if set forth fully herein.

An example of another synthetic material that can be used as a backing layer is silicone. A silicone layer in the form of a porous or microporous membrane or a non-porous film is applied and adhered to a matrix construct. When used in wound healing, the silicone layer can be used to handle and maneuver the matrix construct to a skin wound and seal the wound periphery to enclose the matrix construct to treat the wound. The silicone also forms a moisture barrier to keep the wound from drying. Following successful formation of the healed wound tissue, typically at around 21 days, the silicone is peeled back carefully from the edges of the healed or healing wound with forceps.

Proteins can also be added to bioengineered constructs. Examples of useful extracellular matrix proteins include, but are not limited to, collagen, fibrin, elastin, laminin, and fibronectin, proteoglycans. Fibrinogen, when combined with thrombin, forms fibrin. Hyaluronan (also called hyaluronic acid or hyaluronate) is a non-sulfated glycosaminoglycan distributed widely throughout connective, epithelial, and neural tissues. It is one of the chief components of the extracellular matrix, contributes significantly to cell proliferation and migration and is used to reduce post-operative adhesions. There are multiple types of each of these proteins that are naturally-occurring, as well as types that can be or are synthetically manufactured or produced by genetic engineering. Collagen occurs in many forms and types. The term “protein” further includes, but is not limited to, fragments, analogs, conservative amino acid substitutions, and substitutions with non-naturally occurring amino acids with respect to each named protein. The term “residue” refers to an amino acid (D or L) or an amino acid mimetic that is incorporated into a protein by an amide bond. As such, the amino acid can be a naturally occurring amino acid or, unless otherwise limited, can encompass known analogs of natural amino acids that function in a manner similar to the naturally occurring amino acids (i.e., amino acid mimetics). Moreover, an amide bond mimetic includes peptide backbone modifications well known to those skilled in the art. For example, peptides can be used to enhance cellular effects (e.g., human dermal fibroblast infiltration into a silk scaffold and improve the ability to recruit host cells, such as epithelial cells). Such peptides could be RGD, Gofoger, laminin 1-10, and pronectin. More specifically, laminin 5 and laminin 10 function particularly well to increase epithelial cell infiltration/migration. Peptides can also be used to enhance endothelial cell migration. More particularly, peptides such as thrombin and fibrinogen can enhance endothelial cell migration, especially for indications benefiting from neovascularization.

Cell adhesion molecule can also be incorporated into or onto the polymer matrix to attach the scaffold composition to the local tissue site and prevent diffusion of the bioengineered construct. Such molecules are incorporated into the polymer matrix prior to polymerization of the matrix or after polymerization of the matrix. Examples of cell adhesion molecules include but are not limited to peptides, proteins and polysaccharides such as fibronectin, laminin, collagen, thrombospondin 1, vitronectin, elastin, tenascin, aggrecan, agrin, bone sialoprotein, cartilage matrix protein, fibrinogen, fibrin, fibulin, mucins, entactin, osteopontin, plasminogen, restrictin, serglycin, SPARC/osteonectin, versican, von Willebrand Factor, polysaccharide heparin sulfate, connexins, collagen, RGD (Arg-Gly-Asp) and YIGSR (Tyr-Ile-Gly-Ser-Arg) peptides and cyclic peptides, glycosaminoglycans (GAGs), hyaluronic acid (HA), condroitin-6-sulfate, integrin ligands, selectins, cadherins and members of the immunoglobulin superfamily. Other examples include neural cell adhesion molecules (NCAMs), intercellular adhesion molecules (ICAMs), vascular cell adhesion molecule (VCAM-1), platelet-endothelial cell adhesion molecule (PECAM-1), L1, and CHL1.

### ECM Proteins and Peptides and Role in Cell Function

| | | SEQ. | |
|---|---|---|---|
| | | ID | |
| Protein | Sequence | No: | Role |
| Fibronectin | RGDS | | Adhesion |
| | LDV | | Adhesion |
| | REDV | | Adhesion |
| Vitronectin | RGDV | | Adhesion |
| Laminin A | LRGDN | | Adhesion |
| | IKVAV | | Neurite extension |
| Laminin B1 | YIGSR | | Adhesion of cells, |
| | | | via 67 kD laminin |
| | | | receptor |
| | PDSGR | | Adhesion |
| Laminin B2 | RNIAEIIKDA | | Neurite extension |
| Collagen 1 | RGDT | | Adhesion of most |
| | | | cells |
| | DGEA | | Adhesion of platelets |
| | | | and other cells |
| Thrombospondin | RGD | | Adhesion of most |
| | | | cells |
| | VTXG | | Adhesion of platelets |

Additional examples of suitable cell adhesion molecules are shown below.

Amino Acid Sequences Specific for Proteoglycan Binding from Extracellular Matrix Proteins

| | SEQ. | |
|---|---|---|
| SEQUENCE | ID. NO. | PROTEIN |
| XBBXBX* | | Consensus |
| | | sequence |
| PRRARV | | Fibronectin |
| YEKPGSPPREVVPRPRPGV | | Fibronectin |
| RPSLAKKQRFRHRNRKGYRSQRGHSRGR | | Vitronectin |
| rIQNLLKITNLRIKFVK | | Laminin |

Particularly preferred cell adhesion molecules are peptides or cyclic peptides containing the amino acid sequence arginine-glycine-aspartic acid (RGD) which is known as a cell attachment ligand and found in various natural extracellular matrix molecules. A polymer matrix with such a modification provides cell adhesion properties to the scaffold, and sustains long-term survival of mammalian cell systems, as well as supporting cell growth.

Growth factors can also be introduced into the bioengineered constructs and/or onto the scaffold structures. Such substances include BMP, bone morphogenetic protein; ECM, extracellular matrix proteins or fragments thereof; EGF, epidermal growth factor; FGF-2, fibroblast growth factor 2; NGF, nerve growth factor; PDGF, platelet-derived growth factor; PIGF, placental growth factor; TGF, transforming growth factor, VEGF, vascular endothelial growth factor, MCP1, and IL4. Cell-cell adhesion molecules (cadherins, integrins, ALCAM, NCAM, proteases, Notch ligands) are optionally added to the scaffold composition. Exemplary growth factors and ligands are provided in the tables below.

### Growth Factors Used for Angiogenesis

| Growth factor | Abbreviation | Relevant activities |
|---|---|---|
| Vascular endothelial | VEGF | Migration, proliferation and |
| growth factor | | survival of ECs |
| Basic fibroblast | bFGF-2 | Migration, proliferation and survival |
| growth factor | | of ECs and many other cell types |
| Platelet-derived | PDGF | Promotes the maturation of blood |
| growth factor | | vessels by the recruitment of smooth |
| | | muscle cells |
| Angiopoietin-1 | Ang-1 | Strengthens EC-smooth muscle cell |
| | | interaction |
| Angiopoietin-2 | Ang-2 | Weakens EC-smooth muscle cell |
| | | interaction |
| Placental growth | PIGF | Stimulates angiogenesis |
| factor | | |
| Transforming | TGF | Stabilizes new blood vessels by |
| growth factor | | promoting matrix deposition |

### Growth Factors Used for Wound Healing

| Growth Factor | Abbreviation | Relevant activities |
|---|---|---|
| Platelet-derived | PDGF | Active in all stages of healing |
| growth factor | | process |
| Epidermal growth | EGF | Mitogenic for keratinocytes |
| factor | | |
| Transforming | TGF-β | Promotes keratinocyte migration, |
| growth factor-β | | ECM synthesis and remodeling, and |
| | | differentiation of epithelial cells |
| Fibroblast | FGF | General stimulant for wound healing |
| growth factor | | |

### Growth Factors Used for Tissue-Engineering

| | | Moleular | | Representative supplier |
|---|---|---|---|---|
| Growth factor | Abbreviation | weight (kDa) | Relevant activities | of rH growth factor |
| Epidermal growth | EGF | 6.2 | Proliferation of epithelial, mesenchymal, and | PeproTech Inc. (Rocky |
| factor | | | fibroblast cells | Hill, NJ, USA) |
| Platelet-derived | PDGF-AA | 28.5 | Proliferation and chemoattractant agent for | PeproTech Inc. |
| growth factor | PDGF-AB | 25.5 | smooth muscle cells; extracellular matrix | |
| | PDGF-BB | 24.3 | synthesis and deposition | |
| Transforming | TFG-α | 5.5 | Migration and proliferation of keratinocytes; | PeproTech Inc. |
| growth factor-α | | | extracellular matrix synthesis and deposition | |
| Transforming | TGF-β | 25.0 | Proliferation and differentiation of bone | PeproTech Inc. |
| growth factor-β | | | forming cells; chemoattractant for fibroblasts | |
| Bone morphogenetic | BMP-2 | 26.0 | Differentiation and migration of bone | Cell Sciences Inc. |
| protein | BMP-7 | 31.5 | forming cells | (Norwood, MA, USA) |
| Basic fibroblast | bFGF/FGF- | 17.2 | Proliferation of fibroblasts and initiation of | PeproTech Inc. |
| growth factor | 2 | | angiogenesis | |
| Vascular endothelial | VEGF₁₆₅ | 38.2 | Migration, proliferation, and survival of | PeproTech Inc. |
| growth factor | | | endothelial cells | |

rH, recombinant human

### Immobilized Ligands Used in Tissue Engineering

| Immobilized | | |
|---|---|---|
| ligand* | ECM molecule source | Application |
| RGD | Multiple ECM molecules, | Enhance bone and cartilage tissue |
| | including fibronectin, | formation in vitro and in vivo |
| | vitronectin, laminin, collagen | Regulate neurite outgrowth in vitro |
| | and thrombospondin | and in vivo |
| | | Promote myoblast adhesion, |
| | | proliferation and differentiation |
| | | Enhance endothelial cell adhesion |
| | | and proliferation |
| IKVAV YIGSR | Laminin | Regulate neurite outgrowth in vitro |
| RNIAEIIKDI | | and in vivo |
| Recombinant | Fibronectin | Promote formulation of focal |
| fibronectin | | contacts in pre-osteoblasts |
| fragment | | |
| (FNIII₇₋₁₀) | | |
| Ac-GCRDGPQ- | Common MMP substrates, (e.g. | Encourage cell-mediated proteolytic |
| GIWGQDRCG | collagen, fibronectin, laminin) | degradation, remodeling and bone |
| | | regeneration (with RGD and BMP-2 |
| | | presentation) in vivo |

*Sequences are given in single-letter amino acid code. MMP, matrix metalloproteinase.

In order to enhance blood vessel formation in vivo, devitalized bioengineered constructs can be soaked in proteins such as platelet-derived growth factor (PDGF), fibroblast growth factor (FGF), hepatocyte growth factor/scatter factor (HGF/SF), insulin-like growth factor (IGF), vascular endothelial growth factor (VEGF) and other kinds of pro-angiogenic factors. In one aspect, 50 micrograms of recombinant human PDGF-BB powder was reconstituted in 0.5 ml 4 mM HCl, and then added with an additional 0.5 ml phosphate buffered saline (PBS). The resulting 1 mL solution was used to soak a devitalized bioengineered construct prior to implantation in a full thickness wound in nude and normal mice. In addition, 50 micrograms of recombinant human basic fibroblast growth factor (bFGF) was reconstituted in 1 mL of PBS. Bioengineered constructs were soaked in the 1 mL bFGF solution for 5 minutes prior to implantation in a full thickness wound in nude and normal mice. In another embodiment, 50 micrograms of recombinant human PDGF-BB was reconstituted in 0.5 ml 4 mM HCL and subsequently mixed with 0.5 mL PBS-reconstituted recombinant human bFGF. Bioengineered constructs were soaked in the resulting 1 mL solution for five minutes prior to implantation in a full thickness wound on nude and normal mice. In another embodiment, bioengineered constructs are produced as in Example 30. The conditioned culture medium from any of the multiple feeds over the course of the culture time can be collected. In particular, conditioned culture medium was collected after day 11 and concentrated (e.g. 100-fold). Devitalized bioengineered constructs of the invention were subsequently soaked in the concentrated conditioned media immediately prior to implantation.

Supplements can also be introduced into the chemically defined culture medium in order to selectively enhance desired extracellular matrix attributes and/or to achieve desired in vivo outcomes. Chemically defined culture medium comprises the following:

| Component | Concentration (Volume for 1 L) | |
|---|---|---|
| DMEM | 96.0% (960 mL in 1 L) | |
| L-Glutamine | 1060 | mg/L |
| Hydrocortisone | 0.4 | mg/L |
| Selenious acid | 6.78 | μg/L |
| ITT (2.5 mg/mL Insulin + 2.5 mg/mL | 2 | mL |
| Transferrin + 6.74 ng/mL | | |
| Triiodothyronine) | | |
| EOP ((3.103 g/L monoethalonamine + | 2 | mL |
| 7.06 g/L o-Phosphorylethanolamine) | | |
| EGF | 10.0 | μg/L |
| Mg Ascorbate | 50 | mg/L |
| L-Proline | 213.6 | mg/L |
| Glycine | 101.4 | mg/L |
| Long TGFα | 20 | ng/mL |
| Prostaglandin 2 | 0.038 | μg/mL |

In order to increase the amount of hyaluronic acid (HA) in the bioengineered construct and to enhance new blood vessel formation in vivo, the chemically defined culture medium can be supplemented with 2× long TGFα (40 ng/mL). Additionally, the chemically defined culture medium can further be supplemented with 25 ng/ml of PDGF at day 5, 25 ng/ml of bFGF at day 10, and 25 ng/ml of hepatocyte growth factor (HGF) at day 15. Alternatively, the chemically defined cultured media comprises supplementation with 2× long TGFα (40 ng/mL), 25 ng/ml of bFGF at day 5, 25 ng/ml of PDGF at day 10, and 25 ng/ml of bFGF at day 15. An additional alternative chemically defined media formulation is 2× long TGFα (40 ng/mL), 25 ng/ml of pDGF on day 5, 25 ng/ml bFGF on day 10, and 25 ng/ml of HGF on day 15. Alternatively, bioengineered constructs of the invention can be produced to comprise an elevated amount of sulfated glycosaminoglycans (sGAG) by supplementing the chemically defined culture medium to comprise 10× long TGFα (200 ng/mL). More particularly, when comparing bioengineered constructs produced by supplementing the chemically defined culture media with 10× long TGFα (200 ng/mL) and 1× TGFα (20 ng/mL), approximately 1100 ug sGAG/construct was observed in the bioengineered constructs produced by the medium supplemented with 10× long TGFα (200 ng/mL), as opposed to 600 ug sGAG/construct in bioengineered constructs produced in medium supplemented with 1× TGFα (20 ng/mL). It is to be appreciated that the changes in media supplementations disclosed herein can be used to treat the silk scaffold with or without HDFs seeded thereon without straying from the scope of the invention.

Bioengineered constructs can be treated with a surface modification to enhance the adhesiveness or tissue-attachment properties. The surface modification providing the adhesive “means” can be included on the apical, basal, or both opposing surfaces which functions to increase the binding of a construct when intimately applied to a patient's tissues and organs in vivo. The adhesive-enhancing “means” can be one or more of any of the following: (a) the incorporation of a plurality of self-assembled microstructure and/or nanostructures molded onto and protruding from the bioengineered surface; (b) an added biocompatible and biodegradable adhesive material, such as a film, gel, hydrogel, liquid, or glue, bonded, coated or applied directly onto the bioengineered surface; or, (c) an electrospun sticky fiber matrix that is overlaid or spun onto the bioengineered surface.

The adhesive-enhancing means can be restricted to one outer surface (either basal or apical, depending on the preferred manufacturing design). This adhesive construct can be used for organ repairs, bulking, reinforcing or reconstruction. The adhesive construct is not meant to adhere to surrounding tissues adjacent to the wound but only to adhere directly to surface of the organ in need of healing. However, both basal and apical surfaces can contain an adhesive-enhancing means, either the same or different means on each surface, depending on the intended therapeutic use of the composition (e.g., for purposely holding internal tissues or organs in tight proximity to one another, or alternatively, for adhering a patient's tissue tightly to the surface of an exogengous, implantable therapeutic device or sensor).

Certain manufacturing methods can be used to produce the various embodiments, whether they are made to contain the self-assembled micro- and/or nano-structures or are made to include the biocompatible and biodegradable adhesive materials. For example, the implant's shape can be a patch that is circular, oval, elliptical, triangular, or various sizes of rectangles and squares dependent on its intended therapeutic use (e.g. long, narrow rectangles for certain applications similar to a tape format where the composition has a length substantially larger than its width, for example for bone or other organ wraps, while other uses can require more square-like patches, for example, for hernia repair. The implant can be further trimmed by the surgeon, as necessary, to match the particular size and shape of the patient's defect. Furthermore, the tape or patch can include one or more drugs to discourage bacterial infection, such as colloidal silver or microbial toxins, and to discourage post-surgical bleeding, such as fibrinogen or thrombin. In a further embodiment, the construct could be mitotically inactivated by gamma irradiation, treatment with mitomycin-C, or any other means known in the art prior to shipment which would allow the donor cells to continue to secrete their biological healing factors but would prevent their long term engraftment in the patient host. At least a portion of the adhesive article can have an adhesive strength of equal to or greater than approximately 0.05 Newton per square centimeter of projected area when measured according to ASTM standard D4501, D4541, or D6862-04.

Adhesive means include a plurality of self-assembled microstructures molded onto the basal surface of the bioengineered constructs produced with fibroblasts and/or mesenchymal progenitor cells unit that is formed by the cells and their secreted extracellular matrix which mimic the modified pore surface of culture insert membranes of the bioreactor system. Essentially the plating system surface acts as as a micromold containing numerous engineered cavities or void structures where the cells would settle into these voids upon culturing and then secrete proteins, lipids, GAGs and other matrix factors to fill these voids, thereby creating the protusions or tissue “grippers” covering all or a portion of the basal surfaces of the bioengineered constructs which are formed in mirror image to the nanoscale topography of the plating surfaces upon removal of the bioengineered constructs from the bioreactor. The microfabricated topography of the plating surfaces can be formed using a variety of techniques known in the art, including but not limited to, lithography, nanodrawing, microetching, and photolithography followed by etching or nanomolding. The protrusions can be formed in a variety of shapes and sizes including cones, spikes, cylinders, prisms, pyramids, polygonal, patterned grooves, suction cups, or the shapes that mimic the nanoscale setae and spatulae topography found on the footpads of geckos. The protrusions can include a second, a third or additional sets of protrusions extending from the main protrusions of the bioengineered construct basal or apical surface. The protrusions would be an inherent feature of the bioengineered constructs and can be uniform in their shape and size on a surface or can be arranged in combinations of shapes and sizes, depending on the intended use and level of adhesiveness required. The protrusions can be arranged in various patterns and in various densities on the surface. The density of protrusions, or the number of protrusions per unit area, ranges from approximately 10 protrusions/cm² to approximately 1×10¹⁰ protrusions/cm². The protrusions can be arranged in a pattern, or regularly, irregularly or randomly arranged, depending on the intended application of the tape or patch. In some embodiments the protrusions have an average height of less than approximately 1,000 micrometers. The protrusions can have an average height of from approximately 0.2 μm to approximately 150 μm. The protrusions can have an average tip width of from approximately 0.05 μm to approximately 150 μm. The protrusions can have an average base width of from approximately 0.05 μm to approximately 150 μm. The protrusions can have an average center-to-center pitch of from approximately 0.2 μm to approximately 500 μm. The protrusions can have an average height to base width ratio of from approximately 0.1:1 to approximately 500:1. The protrusions can have an average base width to tip width ratio of from approximately 1000:1 to approximately 0.1:1. In some embodiments, the self-assembled protrusions can be capable of piercing the tissue of the patient upon application by the surgeon.

Alternatively, the adhesive-enhancing means is an adhesive material applied either to the surface of the bioreactor prior to initial plating of the cells, or alternatively, applied directly to the surface of the self-assembled bioengineered constructs after culturing has been completed but before final packaging (i.e., post-liquid growth medium removal but prior to shipment of the units). Important features for adhesives useful in the invention include ones that are biodegradable, biocompatible, flexible, elastic, able to form strong bonds to tissue surfaces (even in moist or wet environments). The adhesive material should be capable of forming a chemical bond with the cell matrix construct's surface, such as a covalent bond or non-covalently through van der Waals, electrostatic, or hydrogen interactions. The adhesive material can be added to the surface of the construct either by spraying, rolling or dipping. A variety of adhesive materials known in the art can be used to form the adhesive surface including, but are not limited to cellulose, carboxymethyl cellulose, hydroxypropyl methyl cellulose or combinations thereof. Other materials for use in the adhesive surface can include but are not limited to poly(glycerol sebacate) (PGS), poly(glycerol sebacate acrylate) (PGSA), poly(lactic-co-glycolic acid) (PLGA), polycaprolactone (PCL), polyglycolide (PGA), polylactic acid (PLA), poly-3-hydroxybutyrate (PHB), phosphoester polyamines, polyurethane, parylene-C, keratin, carbon nanotubes, poly(anhydride), polyvinylpyrrolidone, polypropylene glycol, hyaluronic acid, dextrans, collagen, chitin, chitosan, silk fibroin, glycosaminoglycans, fibrin, fibrinogen or the like.

The adhesive-enhancing means can also be made from nanofibers or microfibers having inherent adhesive properties that are electrospun directly onto the surface of the self-assembled constructs after culturing has been completed but before final packaging (i.e. post-liquid growth medium removal but prior to shipment of the units). The electrospun nanofibers or microfibers can be, but are not limited to, collagen, poly(lactic-co-glycolic acid) (PLGA), polycaprolactone (PCL), polyglycolide (PGA), polylactic acid (PLA), and combinations thereof.

e. Meshed Bioengineered Constructs

Bioengineered constructs can also be meshed prior to grafting to a subject in need of wound care. When used in wound healing, meshing improves conformation to the wound bed and provides a means for draining wound exudate from beneath the graft. The term ‘meshing’ is defined as a mechanical method by which a tissue is perforated with slits to form a net-like arrangement. Meshed constructs can be expanded by stretching the skin so that the slits are opened and then applied to the wound bed. Expanded meshed constructs provide a wound area with maximal coverage. Alternatively, meshed constructs can be applied without expansion, simply as a sheet with an arrangement of unexpanded slits. The meshed construct can be applied alone or with the subject's own skin from another area of the body. Constructs can also have perforations or fenestrations and pores provided by other means. Fenestrations can be applied manually using a laser, punch, scalpel, needle or pin. Bioengineered constructs can also be provided with holes that communicate between both planes of the construct. Holes are perforations that are introduced in a regular or irregular pattern. One could also manually score or perforate a tissue with a scalpel or a needle.

f. Terminally Sterilized Bioengineered Constructs

Constructs can be terminally sterilized using means known in the art. A preferred method for sterilization is by contacting the constructs with sterile 0.1% peracetic acid (PA) treatment neutralized with a sufficient amount of 10 N sodium hydroxide (NaOH), according to U.S. Pat. No. 5,460,962, the disclosure of which is incorporated herein. Decontamination is performed in a container on a shaker platform, such as 1 L Nalge containers, for between 16-20 hours (e.g., 18 hours). Constructs can then be rinsed by contacting them with three volumes of sterile water for 10 minutes each rinse.

Constructs can be sterilized by gamma irradiation. Constructs can be packaged in containers made from material suitable for gamma irradiation and sealed using a vacuum sealer, which were in turn placed in hermetic bags for gamma irradiation between 15.0 and 40.0 kGy. Gamma irradiation significantly, but not detrimentally, decreases susceptibility to construct degradation, Young's modulus and shrink temperature. The mechanical properties after gamma irradiation are still sufficient for use in a range of applications and gamma is a preferred means for sterilizing as it is widely used in the field of implantable medical devices.

### V. Treatment Methods and Medical Uses

Bioengineered constructs, with or without cells, can be delivered to a subject, for example, to treat a damaged or diseased organ or tissue, to repair the organ or tissue and/or to restore its intended functionality. The bioengineered constructs of the invention have properties that, when implanted into a subject in a therapeutically effective amount, induce site-appropriate tissue repair and regeneration. A therapeutically effective amount of a construct can be provided to a subject in one or more administrations or applications. Due to the differentiation potential of mesenchymal progenitor cells, inclusion of these multipotential cell populations will improve the the rate and quality of healing of bone, cartilage, tendon, ligament, muscle, and skin). The bioengineered constructs are angiogenic, anti-inflammatory, osteogenic, adipogenic or fibrogenic, or a combination thereof, when implanted adjacent to, or in contact with, the tissue or organ to be treated as appropriate for that site of implantation.

The bioengineered constructs of the invention have angiogenic properties, meaning that they induce the growth of new blood vessels, which is important for wound healing and formation of granulation tissue of cutaneous wounds and other surgical applications of the bioengineered constructs. Angiogenesis is detected by way of, for example, standard histology techniques (such as by way of αSMA staining) or other assays as disclosed herein.

The bioengineered constructs of the invention have anti-inflammatory properties when implanted, meaning that host inflammatory cell infiltration is minimized so that host cells rather will migrate into the implanted bioengineered construct for bioremodeling of the construct and repair of host tissue. Host cell migration from the host tissues into the implanted bioengineered construct will, as part of the regenerative healing response. Histological techniques can be used to determine the extent of inflammatory cell infiltration and host cell migration. The bioengineered constructs of the invention also have osteogenic properties, meaning that new bone formation will occur at a treatment site. Osteogenesis is measured by detection of new connective and ossifying tissue, higher cell activity detection and turnover of the newly formed tissues. Standard histology techniques and other techniques can be used to measure the cellular effect as well as bone density and bone surface area at the treatment site. The bioengineered constructs are adipogenic, forming new adipose (fat) tissue, when implanted at a treatment site. Fibrogenic properties of the bioengineered constructs may be realized when implanted at a treatment site. Bioengineered constructs of the invention can be used for a variety of human and non-human (i.e., veterinary) therapeutic applications.

The invention includes medical uses and methods for treating subjects in need of wound healing using a bioengineered construct of the invention to treat surgical wounds; burn wounds; chronic wounds; diabetic lower extremity ulcers; venous ulcers; pressure ulcers (with or without negative pressure wound therapy); arterial ulcers; tunneling wounds such as those that tunnel away from a chronic wound cavity; sinuses (e.g., pilonidal, post-surgical dehiscences) and fistulae (e.g., anal, enterocutaneous, vesico-vaginal, oro-antral, broncho-pleural).

Other medical uses and methods for treating subjects in need of treatment include cardiac applications, applications to hard and soft tissues of the oral cavity (e.g., treatment of receded gingival tissue, guided bone regeneration to repair bone defects or deteriorated bone, guided tissue regeneration and repair of connective tissues of the oral cavity),

Additional medical uses and treatment methods for using the bioengineered constructs include cosmetic applications including dermal soft tissue fillers (e.g., contouring for cosmesis), breast reconstruction applications (e.g., augmentation, lift, and/or mastopexy) and neurological applications, such as a dura mater repair patch or a graft for peripheral nerve repair, a wrap for nerve bundles or tube for guided nerve regeneration.

Further uses of the bioengineered constructs include, but are not limited to, the application to suture lines or open wounds to improve the sealing and strength capabilites for certain surgical procedures where leakage of air or fluids would be detrimental to the subject's health and require additional corrective surgical procedures to prevent complications, such as infection, abscess formation, or internal bleeding (e.g., gastric bypass; colostomies; stomach and large and small bowel resections; vascular grafts; vascular implants; coronary artery bypass grafts; abdominoplasty; abdominal surgeries (e.g. laparotomy); Caesarean-sections; tracheostomy sites; catheter implant sites; sealing of pericardium, pleura, and dural trauma); application as a prophylactic treatment to heal or prevent rupture of organs (e.g., vulnerable plaque stabilization; abdominal aortic/aneurysm ruptures; stomach or small intestine ulcer perforations; Crohn's disease; inflammatory bowel disease); “holes” that need filling for cell growth repair (e.g., urinary incontinence; nose or septum repairs; anal fistulas; ostomies; muscle tears; cartilage tears; joint coating material; soft tissue and muscle wall hernia repairs;);

Still further uses of the bioengineered constructs include, but are not limited to bone grafts and repairs (e.g. compound fractures; osteotomies; artificial periosteal membrane; stump covering for limb and appendage amputations; foot and ankle fusions); cardiovascular tissue repair and regeneration (post-myocardial infarction; congestive heart failure); myocardial ischemia; stroke; peripheral arterial disease; neuropathies; coronary artery disease); nerve repair applications; liver regeneration applications (fibrosis; acute, subacute and chronic hepatisis; cirrhosis; fulminant hepatic failure; covering of the outer surface following lobe transplantation); kidney regeneration applications during acute renal failure; surgical wound closures; abdominal surgical adhesion prevention; cardiovascular, salivary duct, or bile-duct stent covering.

The bioengineered constructs can be applied or implanted to a treatment site by contacting it with damaged or diseased tissue, by filling a void in a tissue space or by placement where a subject's tissue does not or no longer exists. Application or implantation of the bioengineered constructs may be achieved through pressure touch directly to an organ's surface, via wrapping circumferentially around the organ, or affixed to the treatment site using surgical adhesvies, sutures or staples. The bioengineered constructs may also be delivered as a flat sheet, rolled, wadded, or injected to a treatment site. The bioengineered construct can be delivered intraoperably during open surgical procedures, percutaneously, or laparoscopically by passing the construct through a cannula to the defect. Regardless of the mode of delivery, the device functions to stimulate regenerative healing processes by locally delivering the repair building blocks and cell signalling compounds at relevant physiologic concentrations, including cells along with their complex array of secreted cytokines, ECM proteins, glycosaminoglycans, lipids, matrix reorganizing enzymes, and collagen materials which can be reorganized to meet the needs of the wounded organ or function to locally recruit the endogenous regenerative cells of the host. Alternatively, the bioengineered constructs can incorporate genetically modified cells that function to deliver local cell-based gene therapy to certain organs of a subject in need thereof The construct can also incorporate a drug to function as a drug delivery vehicle for small molecule therapeutics, biological therapeutics or pharmaceuticals for the internal, local, sustained, slow-release delivery of therapeutics to a subject in need thereof.

The following examples are provided to better explain the practice of the present invention and should not be interpreted in any way to limit the scope of the present invention. Those skilled in the art will recognize that various modifications can be made to the methods described herein while not departing from the spirit and scope of the present invention.

### EXAMPLES

### Example 1

### Formation of a Collagenous Matrix by Human Neonatal Foreskin Fibroblasts

Human neonatal foreskin fibroblasts (originated at Organogenesis, Inc. Canton, Mass.) were seeded at 5×10⁵ cells/162 cm² tissue culture treated flask (Costar Corp., Cambridge, Mass., cat #3150) and grown in growth medium. The growth medium consisted of: Dulbecco's Modified Eagle's medium (DMEM) (high glucose formulation, without L-glutamine, BioWhittaker, Walkersville, Md.) supplemented with 10% newborn calf serum (NBCS) (HyClone Laboratories, Inc., Logan, Utah) and 4 mM L-glutamine (BioWhittaker, Walkersville, Md.). The cells were maintained in an incubator at 37±1° C. with an atmosphere of 10±1% CO₂. The medium was replaced with freshly prepared medium every two to three days. After 8 days in culture, the cells had grown to confluence, that is, the cells had formed a packed monolayer along the bottom of the tissue culture flask, and the medium was aspirated from the culture flask. To rinse the monolayer, sterile-filtered phosphate buffered saline was added to the bottom of each culture flask and then aspirated from the flasks. Cells were released from the flask by adding 5 mL trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the suspension to stop the action of the trypsin-versene. The cell suspension was removed from the flasks and evenly divided between sterile, conical centrifuge tubes. Cells were collected by centrifugation at approximately 800-1000×g for 5 minutes.

Cells were resuspended using fresh medium to a concentration of 3.0×10⁶ cells/ml, and seeded onto 0.4 micron pore size, 24 mm diameter tissue culture treated inserts (TRANSWELL®, Corning Costar) in a six-well tray at a density of 3.0×10⁶ cells/insert (6.6×10⁵ cells/cm²). The cells were maintained in an incubator at 37±1° C. with an atmosphere of 10±1% CO₂ and fed fresh production medium every 2 to 3 days for 21 days. The production medium comprised: a 3:1 base mixture of DMEM and Hams F-12 medium (Quality Biologics Gaithersburg, Md.), 4 mM GlutaMAX-1™ (Gibco BRL, Grand Island, N.Y.) and additives to a resultant concentration of: 5 ng/ml human recombinant epidermal growth factor (Upstate Biotechnology Lake Placid, N.Y.), 2% newborn calf serum (Hyclone, Logan, Utah), 0.4 μg/m1 hydrocortisone (Sigma St. Louis, Mo.), 1×10⁻⁴M ethanolamine (Fluka, Ronkonkoma, N.Y. ACS grade), 1×10⁻⁴ M o-phosphoryl-ethanolamine (Sigma, St. Louis,), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Chemicals USA, Inc.#013-12061), 0.2 μg/ml L-proline (Sigma, St. Louis, Mo.), 0.1 μg/ml glycine (Sigma, St. Louis, Mo.) and 0.05% poly-ethylene glycol (PEG) 3400-3700 MW (cell culture grade) (Sigma, St. Louis, Mo.).

Samples for histological analysis were taken at days 7, 14 and 21 and fixed in formalin, then embedded in paraffin. The formalin fixed samples were embedded in paraffin and 5 micrometer section were stained with hematoxylin-eosin (H&E) according to procedures known in the art. Using H&E stained slides, thickness measurements were made to ten randomly picked microscopic fields utilizing a 10× eyepiece loaded with a 10 mm/100 micrometer reticle.

Results for two different cell strains of human dermal fibroblasts are summarized in Table 1, which shows the thickness of the cell-matrix construct as it develops.

TABLE 1
Thickness (microns)

| | Day 0 | Day 7 | Day 14 | Day 21 |
|---|---|---|---|---|
| B119 | 0 | 30.33 ± 2.61 | 63.33 ± 4.40 | 84.00 ± 4.67 |
| Average | | | | |
| (n = 3) | | | | |
| B156 | 0 | 42.00 ± 5.14 | 63.85 ± 4.50 | 76.25 ± 8.84 |
| Average | | | | |
| (n = 4) | | | | |

Samples were also submitted for collagen concentration analysis on days 7, 14, and 21. Collagen content was estimated by employing a colorimetric assay for hydroxyproline content known in the art (Woessner, 1961). At those same timepoints cell number was also determined. Table 2 is a summary of collagen concentration and Table 3 is a summary of the cell data from cell-matrix constructs produced from two different cell strains (B156 and B119) using the procedure described above.

TABLE 2
Collagen (μg/cm²)

| | Day 0 | Day 7 | Day 14 | Day 21 |
|---|---|---|---|---|
| B119 | 0 | 93.69 ± 22.73 | 241.66 ± 21.08 | 396.30 ± 29.38 |
| Average | | | | |
| (n = 3) | | | | |
| B156 | 0 | 107.14 ± 17.16 | 301.93 ± 23.91 | 457.51 ± 25.00 |
| Average | | | | |
| (n = 3) | | | | |

TABLE 3
Cells (cells/cm²)

| | Day 0 | Day 7 | Day 14 | Day 21 |
|---|---|---|---|---|
| B119 | 6.6 × 10⁵ | 11.8 ± 4.4 × 10⁵ | 11.4 ± 1.7 × 10⁵ | 13.9 ± 1.2 × 10⁵ |
| Average | | | | |
| (n = 3) | | | | |
| B156 | 6.6 × 10⁵ | 13.1 ± 0.5 × 10⁵ | 14.0 ± 2.1 × 10⁵ | 17.1 ± 1.7 × 10⁵ |
| Average | | | | |
| (n = 3) | | | | |

Samples of the human cell derived dermal matrix from days 7, 14, and 21 were analyzed by delayed reduction SDS-PAGE to determine collagen composition revealing type I and type III collagen alpha bands in the samples.

Biochemical characteristics of the dermal matrix were determined using immunohistochemical methods. Fibronectin identification was carried out on paraffin fixed sections using the Zymed Histostain strepavidin-biotin system (Zymed Laboratories Inc., South San Francisco, Calif.). Tenascin presence was determined by primary anti-tenascin antibody staining (Dako, Carpintheria, Calif.) followed by anti-mouse horseradish peroxidase labeled antibody (Calbiochem) as a secondary antibody. Samples were visualized by applying diaminobenzyne (Sigma St. Louis, Mo.) and counterstained with Nuclear Fast red.

Glycosaminoglycan (GAG) quantification was performed on 21 day samples using the previously described method (Farndale, 1986). The assay showed the presence of 0.44 grams of GAG per cm² in a sample of human cell derived dermal matrix taken 21 days post seeding.

### Example 2

### In Vitro Formation of a Collagenous Matrix By Human Neonatal Foreskin Fibroblasts in Chemically Defined Medium

Human neonatal foreskin fibroblasts were expanded using the procedure described in Example 1. Cells were then resuspended to a concentration of 3×10⁶ cells/ml, and seeded on to 0.4 micron pore size, 24 mm diameter tissue culture treated membrane inserts in a six-well tray at a density of 3.0×10⁶ cells/TW (6.6×10⁵ cells/cm²). These cells were then maintained as Example 1 with newborn calf serum omitted from the media throughout. More specifically the medium contained: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 0.4 μg/ml hydrocortisone (Sigma, St. Louis, Mo.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo.). Samples were checked at day 7, 14, and 21 for collagen concentration and cell number using described procedures. Results are summarized in tables 4 (cell number) and 5 (collagen). Samples were also formalin fixed and processed for hemotoxylin and eosin staining for light microscope analysis as described in Example 1. Histological evaluation demonstrated that the constructs grown in defined medium was similar to those grown in the presence of 2% newborn calf serum. Samples also stained positively for fibronectin, using procedure described in Example 1.

TABLE 4
Collagen (μg/cm²)

| | Day 0 | Day 7 | Day 14 | Day 21 |
|---|---|---|---|---|
| Average amount of | 0 | 107.63 ± 21.96 | 329.85 ± 27.63 | 465.83 ± 49.46 |
| collagen in each | | | | |
| construct (n = 3) | | | | |

TABLE 5
Cells (cells/cm²)

| | Day 0 | Day 7 | Day 14 | Day 21 |
|---|---|---|---|---|
| Average number | 6.6 × 10⁵ | 7.8 ± | 9.6 ± | 1.19 ± 2.1 × 10⁵ |
| of cells in each | | 2.2 × 10⁵ | 2.5 × 10⁵ | |
| construct (n = 3) | | | | |

Besides endogenously produced fibrillar collagen, decorin and glycosaminoglycan were also present in the cell-matrix construct.

### Example 3

### In Vitro Formation of a Collagenous Matrix by Human Achilles Tendon Fibroblasts

Cell-matrix constructs were formed using the same method described in Example 1 replacing the human neonatal foreskin fibroblasts with human Achilles tendon fibroblasts (HATF.). Following 21 days in production medium, samples were also submitted for H&E staining and thickness determination using the procedure described in Example 1. The resulting construct was visualized as a cell matrix tissue like construct with a thickness of 75.00±27.58 microns (n=2). Endogenously produced fibrillar collagen, decorin and glycosaminoglycan were also present in the construct.

### Example 4

### In Vitro Formation of a Collagenous Matrix by Transfected Human Neonatal Foreskin Fibroblasts

Transfected human dermal fibroblasts were produced using the following procedure. One vial of jCRIP-43 platelet derived growth factor (PDGF) viral producers (Morgan, J, et al.) was thawed, and the cells were seeded at 2×10⁶ cells/162 cm² flask (Corning Costar, Cambridge, Mass.). These flasks were fed a growth medium, and maintained in an incubator at 37±1° C. with an atmosphere of 10±1% CO₂. The growth medium consisted of: Dulbecco's modified Eagle's medium (DMEM) (high glucose formulation, without L-glutamine, BioWhittaker, Walkersville, Md.) supplemented with 10% newborn calf serum (HyClone Laboratories, Inc., Logan, Utah) and 4 mM L-glutamine (BioWhittaker, Walkersville, Md.). On the same day, 1 vial of human neonatal foreskin fibroblast (HDFB156) was also thawed and plated at 1.5×10⁶ cells/162 cm² flask (Corning Costar, Cambridge, Mass.). After three days the jCRIP PDGF-43 viral producers were fed with fresh growth medium. The HDFB156 were fed with the above growth medium plus 8 μg/ml polybrene (Sigma, St. Louis, Mo.). The next day the HDFB156′s cells were infected as follows. The spent medium from the jCRIP PDGF-43 viral producers was collected and filtered through a 0.45 micron filter. 8 μg/ml polybrene was added to this filtered spent medium. The spent medium was then placed on the HDF. On the next two days the HDF were fed fresh growth medium. The day after the HDF were passed from p5 to p6 and seeded at a density of 2.5×10⁶ cells/162 cm² flask (Corning Costar, Cambridge, Mass.). Cells were passed as follows; spent medium was aspirated off. The flasks were then rinsed with a phosphate buffered saline to remove any residual newborn calf serum. Cells were released from the flask by adding 5 mL trypsin-versene to each flask and gently rocking to ensure complete coverage of the monolayer. Cultures were returned to the incubator. As soon as the cells were released, 5 mL of SBTI (Soybean Trypsin Inhibitor) was added to each flask and mixed with the suspension to stop the action of the trypsin-versene. The cell/Trypsin/SBTI suspension was removed from the flasks and evenly divided between sterile, conical centrifuge tubes. Cells were collected by centrifugation at approximately 800-1000×g for 5 minutes.). The cells were resuspended in the growth media for seeding at the density listed above. After two days the cells were fed fresh growth medium. The following day the cells were harvested as above, and diluted to a density of 1.5×10⁶ cells/ml in growth medium containing 10% newborn calf serum (NBCS) with 10% dimethyl sulfoxide (DMSO) (Sigma, St. Louis, Mo.). The cells were then frozen 1 ml/cryovial at about −80 ° C.

Production of the collagenous matrix for this example utilize the same procedure as Examples 1 and 3, replacing the human neonatal foreskin fibroblasts with human neonatal foreskin fibroblasts transformed to produce high levels of platelet derived growth factor (PDGF) as described above. Samples were taken for H&E staining as described above on day 18 post seeding. Samples were also stained using the avidin-biotin methods for the presence of fibronectin listed in Example 10. Samples were taken on day 18 post seeding for H&E staining as described in Example 1, and exhibited a similar cell-matrix gross appearance to that described in Example 1, with a measured thickness of 123.6 microns (N=1). PDGF output of the transfected cells in the cell-matrix construct was measured to be 100 ng/mL by ELISA throughout the duration of the culture (18 days) while control output of PDGF was undetectable.

### Example 5

### In Vitro Formation Of A Matrix By Human Corneal Keratocytes

Human corneal keratocyte cells (originated at Organogenesis, Inc. Canton, Mass.) were used in the production of a stromal construct of cornea. Confluent cultures of human keratocytes were released from their culture substrates using trypsin-versene. When released, soybean trypsin inhibitor was used to neutralize the trypsin-versene, the cell suspension was centrifuged, the supernatant discarded and the cells were then resuspended in base media to a concentration of 3×10⁶ cells/ml. Cells were seeded onto 0.4 micron pore size, 24 mm diameter tissue culture treated transwells in a six-well tray at a density of 3.0×10⁶ cells/TW (6.6×10⁵ cells/cm²). These cultures were maintained overnight in seed medium. The seed medium was composed of: a base 3:1 mixture of Dulbecco's Modified Eagle's Medium (DMEM) and Hams F-12 Medium (Quality Biologics Gaithersburg, Md. cat.), 4 mM GlutaMAX (Gibco BRL, Grand Island, N.Y.) and additives: 5 ng/ml human recombinant epidermal growth factor (EGF) (Upstate Biotechnology Lake Placid, N.Y.), 0.4 μg/ml hydrocortisone (Sigma St. Louis, Mo.), 1×10⁻⁴ M ethanolamine (Fluka, Ronkonkoma, N.Y.), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.). Following this the cultures were fed fresh production medium. The production medium was composed of: a base 3:1 mixture of DMEM, Hams F-12 medium (Quality Biologics Gaithersburg, Md.), 4 mM GlutaMAX (Gibco BRL., Grand Island, N.Y.) and additives: 5 ng/ml Human Recombinant Epidermal growth factor (Upstate Biotechnology Lake Placid, N.Y.), 2% newborn calf serum (Hyclone, Logan, Utah), 0.4 μg/ml hydrocortisone (Sigma, St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis,), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO pure chemical company), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo., cell culture grade).

The cells were maintained in an incubator at 37±1° C. with an atmosphere of 10%±1% CO₂ and fed fresh production medium every 2-3 days for 20 days (for a total of 21 days in culture. After 21 days in culture, the keratocytes had deposited a matrix layer of about 40 microns in thickness, as measured by the method described in Example 1. Endogenously produced fibrillar collagen, decorin and glycosaminoglycan were also present in the cell-matrix construct.

### Example 6

### In Vitro Formation of a Collagenous Matrix by Human Neonatal Foreskin Fibroblasts Seeded in Production Media

Human neonatal foreskin fibroblasts (originated at Organogenesis, Inc. Canton, Mass.) were seeded at 1×10⁵ cells/0.4 micron pore size, 24 mm diameter tissue culture treated carriers in a six-well tray (TRANSWELL®, Costar Corp. Cambridge, Mass.) and grown in growth medium. The growth medium consisted of: Dulbecco's Modified Eagle's medium (DMEM) (high glucose formulation, without L-glutamine, BioWhittaker, Walkersville, Md.) supplemented with 10% newborn calf serum (HyClone Laboratories, Inc., Logan, Utah) and 4 mM L-Glutamine (BioWhittaker, Walkersville, Md.). The cells were maintained in an incubator at 37±1° C. with an atmosphere of 10±1% CO₂. The medium was replaced every two to three days. After 9 days in culture the medium was aspirated from the culture dish, and replaced with production medium. The cells were maintained in an incubator at 37±1 ° C. with an atmosphere of 10±1% CO₂ and fed fresh production medium every 2-3 days for 21 days. The production medium was composed of: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 2% newborn calf serum (Hyclone, Logan, Utah), 0.4 μg/ml hydrocortisone (Sigma St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis,), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Pure Chemical Company), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo., cell culture grade).

Samples were taken at day 21 and fixed in formalin, then embedded in paraffin. The formalin fixed samples were embedded in paraffin and 5 micrometer section were stained with hematoxylin-eosin (H&E) according techniques routinely used in the art. Using H&E stained slides, measurements were made at ten randomly picked microscopic fields utilizing a 10× Eyepiece (Olympus America Inc., Melville, N.Y.) loaded with a 10 mm/100 micrometer reticle (Olympus America Inc., Melville, N.Y.). The constructs created using this method are similar in structure and biochemical composition to those created with Example 1, and have a measured thickness of 82.00±7.64 microns.

### Example 7

### In Vitro Formation of a Collagenous Matrix by Pig Dermal Fibroblasts

Pig Dermal Fibroblasts (originated at Organogenesis, Inc. Canton, Mass.) were seeded at 5×10⁵ cells/162 cm² tissue culture treated flask (Costar Corp., Cambridge, Mass. cat #3150) and grown in growth medium as described below. The growth medium consisted of; Dulbecco's modified Eagle's medium (DMEM) (high glucose formulation, without L-glutamine, BioWhittaker, Walkersville, Md.) supplemented with 10% fetal calf serum (HyClone Laboratories, Inc., Logan, Utah) and 4 mM L-glutamine (BioWhittaker, Walkersville, Md.). The cells were maintained in an incubator at 37±1° C. with an atmosphere of 10%±1% CO₂. The medium was replaced every two to three days. Upon confluence, that is the cells had formed a packed layer at the bottom of the tissue culture flask, the medium was aspirated from the culture dish. To rinse the monolayer, sterile-filtered phosphate buffered saline was added to the monolayer and then aspirated from the dish. Cells were released from the flask by adding 5 ml trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the cell suspension to stop the action of the trypsin-versene. The suspension was removed from the flasks and evenly divided between sterile, conical centrifuge tubes. Cells were collected by centrifugation at approximately 800-1000×g for 5 minutes. Cells were resuspended and diluted to a concentration of 3×10⁶ cells/ml, and seeded onto 0.4 micron pore size, 24 mm diameter tissue culture treated transwells in a six-well tray at a density of 3.0×10⁶ cells/TW (6.6×10⁵ cells/cm²). Cells were maintained overnight in a seed medium. The seed medium consisted of; a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology Lake Placid, N.Y.), 0.4 μg/ml hydrocortisone (Sigma St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis,), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Pure Chemical Company), 0.2 μg/ml L-proline (Sigma, St. Louis, Mo.), and 0.1 μg/ml glycine (Sigma, St. Louis, Mo.). The cells were maintained in an incubator at 37±1° C. with an atmosphere of 10±1% CO₂ and fed fresh production medium every 2-3 days for 7 days. The production medium was composed of: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 2% newborn calf serum (Hyclone, Logan, Utah), 0.4 μg/ml hydrocortisone (Sigma St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis,), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Pure Chemical Company), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo.) cell culture grade. After 7 days the media was replaced with production medium without newborn calf serum. This media was fed fresh to the cells every 2-3 days for 20 more days, for a total of 28 days in culture.

Samples were taken at day 21 and fixed in formalin, then embedded in paraffin. The formalin fixed samples were embedded in paraffin and 5 micrometer section were stained with hematoxylin-eosin (H&E) according to techniques customarily used in the art. Using H&E stained slides, measurements were made at ten randomly picked microscopic fields utilizing a 10× Eyepiece (Olympus America Inc., Melville, N.Y.) loaded with a 10 mm/100 micrometer reticle (Olympus America Inc., Melville, N.Y.). The sample exhibited a structure composed of cells and matrix with a measured thickness of 71.20±9.57 microns. Besides endogenously produced fibrillar collagen, decorin and glycosaminoglycan were also present in the cell-matrix construct.

### Example 8

### In Vitro Formation of a Collagenous Matrix by Human Neonatal Foreskin Fibroblasts in Chemically Defined Medium

Human neonatal foreskin fibroblasts were expanded using the procedure described in Example 1. Cells were then resuspended to a concentration of 3×10⁶ cells/ml, and seeded on to 0.4 micron pore size, 24 mm diameter tissue culture treated membrane inserts in a six-well tray at a density of 3.0×10⁶ cells/TW (6.6×10⁵ cells/cm²). Cells in this example were cultured in chemically defined medium throughout.

The medium contained: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 μg/ml L-proline (Sigma, St. Louis, Mo.), 0.1 μg/ml glycine (Sigma, St. Louis, Mo.).

To the basic medium above, other components were added in these separate Conditions:
1. 5 μg/ml insulin (Sigma, St. Louis, Mo.), 0.4 μg/ml hydrocortisone (Sigma, St. Louis, Mo.), 0.05% poly-ethylene glycol (PEG) (Sigma, St. Louis, Mo.).
2. 5 μg/ml insulin (Sigma, St. Louis, Mo.), 0.4 μg/ml hydrocortisone (Sigma, St. Louis, Mo.).
3. 375 μg/ml insulin (Sigma, St. Louis, Mo.), 6 μg/ml hydrocortisone (Sigma, St. Louis, Mo.).

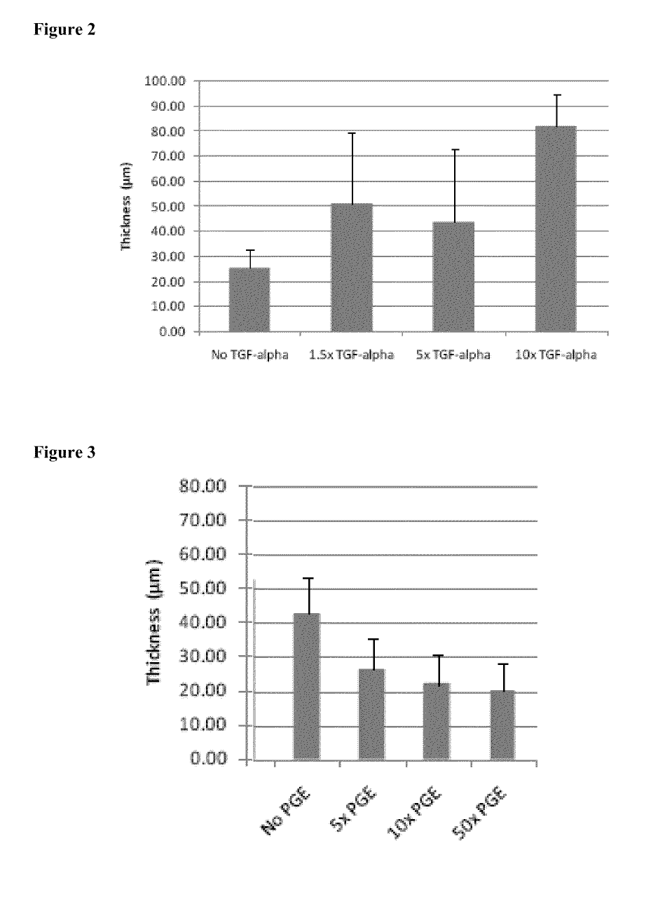

Samples were formalin fixed and processed for hemotoxylin and eosin staining for light microscope analysis. Visual histological evaluation demonstrated that the Condition 2 lacking PEG demonstrated a comparably similar matrix as Condition 1 containing PEG. Biochemical analysis measuring the collagen content of the construct showed nearly the same amount of collagen in both: 168.7±7.98 μg/cm² for Condition 1 with PEG as compared to 170.88±9.07 μg/cm² for Condition 2 without PEG. Condition 3 containing high levels of insulin and hydrocortisone showed a higher expression of matrix, including collagen, at a timepoint earlier than the other two conditions. Besides endogenously produced fibrillar collagen, decorin and glycosaminoglycan were also present in the cell-matrix constructs in all Conditions. The cultured dermal construct formed by the method of Condition 2 of this Example is shown in FIG. 2. Shown in FIG. 2 is a photomicrograph of a fixed, paraffin embedded, hematoxylin and eosin stained section of a cell-matrix construct formed from cultured human dermal fibroblasts in chemically defined medium at 21 days. The porous membrane appears as a thin translucent band below the construct and it can be seen that the cells grow on the surface of the membrane and do not envelope in integrate the membrane with matrix.

FIG. 3 shows transmission electron microscope (TEM) images of two magnifications of cultured dermal construct formed by the method of Condition 2 of this Example at 21 days. FIG. 3A is a 7600× magnification showing alignment of endogenous collagen fibers between the fibroblasts. FIG. 3B is a 19000× magnification of fully formed endogenous collagen fibers demonstrating fibril arrangement and packing

In all Conditions of this Example, the cultured dermal constructs formed comprise dermal fibroblasts and endogenously produced matrix. All have fully formed collagen fibrils in packed organization arranged between the cells. Their fibrous qualities, thickness, and cohesive integrity give the construct considerable strength to allow it to be peelably removed from the culture membrane and handled as it is transferred to a subject to be treated with the construct, as in a graft or implant.

### Example 9

### Formation of a Collagenous Matrix by Human Buccal Fibroblasts

The purpose of this experiment is to produce a cell-matrix construct from buccal fibroblasts isolated from human cheek tissue. Buccal were cultured in T-150 flasks in DMEM containing 10% NBCS medium. After 7 days, to expand the number of cells further, buccal cells were harvested and passaged into nine T-150 flasks at 4.0×10⁶ cells in DMEM containing 10% NBCS medium and cultured until confluence at which time the cells were harvested.

To harvest the cells, the medium was aspirated from the culture flask. To rinse the monolayer, sterile-filtered phosphate buffered saline was added to the bottom of each culture flask and then aspirated from the flasks. Cells were released from the flask by adding 5 mL trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the suspension to stop the action of the trypsin-versene. The cell suspension was removed from the flasks and evenly divided between sterile, conical centrifuge tubes. Cells were collected by centrifugation at approximately 800-1000×g for 5 minutes.

Cells were resuspended using fresh medium to a concentration of 3.0×10⁶ cells/ml, and seeded onto 0.4 micron pore size, 24 mm diameter tissue culture treated inserts (TRANSWELL®, Corning Costar) in a six-well tray at a density of 3.0×10⁶ cells/insert (6.6×10⁵ cells/cm²). The cells were maintained in an incubator at 37±1° C. with an atmosphere of 10±1% CO₂ and fed medium containing: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 0.4 μg/ml hydrocortisone (Sigma, St. Louis, Mo.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo.).

At day 1 post seeding, medium was replaced with Serum Free Production Media, exchanged every 2-3 days for 21 days. At day 21, samples were fixed in formalin for histology. Three samples were used for protein and collagen production analysis.

Collagen production for 24 mm diameter constructs averaged 519 μg per construct after 21 days in culture. Total protein production for 24 mm diameter constructs averaged 210 μg per construct after 21 days in culture. Morphologically, the buccal fibroblast cell-matrix construct, a cultured tissue construct of oral connective tissue, showed buccal fibroblasts surrounded by matrix while physically, the construct had physical bulk and integrity.

### Example 10

### Methods for Termination of Fibroblasts in Endogenously Produced Matrix Constructs to Form Devitalized Cell-Matrix Constructs

Termination of fibroblasts in endogenously produced cell-matrix constructs were assayed using the alamarBlue™ assay. The alamarBlue™ assay incorporates an oxidation-reduction indicator that changes in color in response to chemical reduction of growth medium as a direct result from cell metabolism. Metabolic activity from the cells will result in the reduction of alamarBlue™ to a reddish or pink color as exposure time is increased. Lack of viable cells should result in little or no color change.

Twelve 24 mm diameter matrix constructs made according to the method of Example 1 (cell-matrix construct) were grown on 24 mm culture inserts having 0.4 micron pores with each culture insert residing in a deep-well tray for 25 days. Four methods of fibroblast termination (n=3 for each condition) were carried out overnight: (1) air-drying at ambient room temperature and humidty; (2) rinsing in 100% ethanol; (3) snap freezing followed by air drying; and, (4) lyophilization. The 24 mm membrane culture inserts were transferred back to the original 6-well plates. Each well contained 11.0 mL of Dulbecco's modified Eagle's medium (DMEM) (high glucose formulation, without L-glutamine, BioWhittaker, Walkersville, Md.) with 10% AlamarBlue™. Media was sampled from each well at 8 hours and 24 hours in 200 μL samples, which were taken and aliquotted into a 96-well plate and stored at 2-8° C. After the second time point sample was taken, the samples in the 96-well plate were read on a plate reader at two wavelengths. The percent of AlamarBlue™ reduction was calculated from the absorbance values given by the plate reader.

After 24 hours of incubation, all conditions tested did not show significant metabolic activity suggesting that the cells in each cell-matrix construct had successfully been terminated using these termination methods. The results of this example were devitalized single-layer matrix constructs that may be used in the fabrication of multilayer, or tubular, or complex bioengineered constructs of the invention. The devitalization methods of this example may be applied to any of the living matrix constructs of the previous examples to arrive at a devitalized matrix construct.

### Example 11

### Crosslinking of Human Cell Derived Dermal Matrix

Two 75 mm diameter cell-matrix constructs made according to the method of Example 1 by culturing them on 75 mm diameter, 0.4 micron porous polycarbonate membranes. One cell-matrix construct was devitalized by air drying at room temperature and humidity and the other was devitalized by immersing it overnight in 100% ethanol and then air-drying to allow the ethanol to evaporate from the cell-matrix construct. Both cell-matrix constructs were then rehydrated using a volume of sterile water for injection. To form a 2-layer construct, the cell-matrix constructs were peeled from their respective membranes using curved tweezers and superimposed by layering onto each other using on a 100 mm culture dish lid. Approximately 15 mL of 1.0 mM EDC solution was added to the layered cell-matrix construct and allowed to cross-link overnight, or approximately 15-18 hours. The 2-layer construct was removed from the EDC solution, rinsed in sterile water and air-dried to result in a 2-layer, crosslinked, devitalized cell-matrix construct, endogenously produced by human fibroblast cells.

The construct was then re-hydrated. When rehydrated, the 2-layer construct quickly gained water mass and was smooth in texture. The rehydration process was nearly instantaneous, taking under a minute. The 2-layer construct looked and handled as a single piece of tissue. The construct was cut into four strips roughly 1.25×5 cm then tested on an Instron mechanical testing machine for strength. The average strength for each strip recorded by the Instron machine was 4.9 N.

### Example 12

### Layering and Cross-Linking Devitalized Tissue Constructs to Form Multilayered Constructs

Sixteen matrix constructs of were grown on 75 mm diameter culture insert membranes having pores of about 0.4 μm using methods and materials substantially similar to Example 1. To devitalize them, the tissue constructs were air-dried overnight in ambient room temperature and humidity to ensure fibroblast termination. The devitalized tissue constructs were then hydrated with water for injection (WFI) and the tissue constructs were peeled from each membrane using curved tweezers or blunt forceps. A devitalized tissue construct was fully spread out onto 100 mm culture dish lids (as a convenient working surface) and subsequent layers were added by superimposing the devitalized tissue constructs on top of one another to achieve 2 or 3 layers. The layers were allowed to air-dry in ambient room temperature and humidity overnight, or between about 15-18 hours. After drying, the superimposed layers adhered to each other. The adhered layers were hydrated and then superimposed on other adhered layers and then again allowed to dry overnight, or between about 15-18 hours. From the sixteen single layer devitalized tissue constructs, a 10-layer, a 5-layer and a single layer construct were formed with the layers of the 10-layer and 5-layer constructs adhered to each other by the drying and rehydration process. The constructs were then crosslinked with a crosslinking agent. The constructs were in vessels and to each vessel was added approximately 10 mL of 1.0 mM EDC in WFI solution. The layered devitalized ECM tissue constructs were allowed to cross-link overnight (approximately between 15-18 hours). The tissue constructs were removed from the EDC solution, rinsed with WFI and air-dried in ambient room temperature and humidity one final time resulting in a crosslinked, multilayer, devitalized human cell-derived tissue construct.

The two multi-layered constructs were cut into three strips roughly 1.25×5 cm then tested on an Instron mechanical testing machine to measure strength. The normalized tensile strength per layer of the 5 layer tissue construct was determined to be 1.425 N/layer ; for the 10 layer tissue construct the tensile strength was 1.706 N/layer.

The combination of air-drying, layering, and EDC cross-linking the non-cellular ECM tissue constructs was successful at giving a strength and uniformity to the non-cellular ECM. Non-cellular ECM tissue constructs can successfully be layered, dried and rehydrated. The handling and shape of the non-cellular ECM tissue constructs did not diminish from multiple drying and rehydration steps. Increasing the number of non-cellular ECM tissue construct layers to five and as many as ten increased the overall strength of the construct as well as maintained the shape of the tissue after being cut to size.

### Example 13

### Fabrication of Multi-Layer Cell-Matrix Constructs Crosslinked Using Transglutaminase or Transglutaminase/EDC

Devitalized cell-matrix constructs were layered and crosslinked using transglutaminase or transglutaminase followed with EDC in MES buffer to bond the layers together. Transglutaminase (“TGM”) is a collective term for a family of naturally occurring enzymes that act to link proteins. TGM catalyzes covalent bond formation between free amine groups and γ-carboxamide group specifically acting on lysine and glutamine. There are several forms of TGM, but the type focused on in this experiment will be Activa™ food-grade TGM, which is made through a fermentation process and is used as a binding agent. It comes ready to use a dry powder but can also be mixed as a slurry or solution.

Twelve cell-matrix constructs were grown in 75 mm diameter culture inserts having 0.4 μm porous membranes and cultured for 25 days according methods and using materials substantially similar to those presented in Example 1. The cell-matrix constructs were devitalized by air-drying them overnight, approximately 15-18 hours, at ambient room temperature and humidity. The dried, devitalized cell-matrix constructs were hydrated with sterile water for injection (WFI) and the constructs were peeled from their respective membrane using blunt forceps. Cell-matrix constructs were fully spread out onto a 100 mm culture dish with superimposed cell-matrix constructs layered over them to form three 4-layer constructs. When layering, each cell-matrix layer was contacted with 5.0 mL TGM solution of varying concentration (the concentrations for each 4-layer construct were 28 U, 280 U, and 700 U) added in between individual layers to ensure that the entire cell-matrix layer was treated with crosslinking agent. The cell-matrix constructs were layered loosely in cross-linking solution to evaluate fusing of layers without application of pressure or a drying step. The three 4-layer cell-matrix constructs were placed into a 40° C. incubator overnight, or approximately 15-18 hours. The three 4-layer cell-matrix constructs were removed from the 40° C. incubator and allowed to air-dry at ambient room temperature and humidity. After fully air-drying, the cell-matrix constructs were hydrated with sterile WFI. The cell-matrix constructs were cut in half with half of each construct then treated with 1.0 mM EDC in MES buffer and allowed to cross-link overnight at 4° C. The other halves of each construct that were crosslinked with TGA alone were also stored overnight at 4° C. in WFI. The following day all 6 pieces were tested for tensile strength and suture retention on the Instron machine.

Although results varied from sample to sample, generally, all conditions yielded 4-layer, bonded constructs of devitalized endogenously produced cell-matrix layers having comparable strength per layer measures when comparing TGA treatment alone against TGA/EDC crosslinked and bonded constructs.

### Example 14

### Fabrication of Multi-Layer Cell-Matrix Constructs Crosslinked Using Food-Grade Transglutaminase or Recombinant Transglutaminase

Two forms of transglutaminase (TGM) on multilayered, devitalized cell-matrix constructs were compared. The forms of the enzyme compared were Activa™ food-grade and recombinant human transglutaminase.

Thirty-two cell-matrix constructs were grown in 75 mm diameter culture inserts having 0.4 μm porous membranes and cultured for 25 days according to methods and using materials substantially similar to those presented in Example 1. All cell-matrix constructs were devitalized by air drying in ambient room temperature and humidity. The dried, devitalized single-layer cell-matrix constructs were hydrated 24 hours later with sterile water for injection (WFI) and the constructs were then peeled from their respective membranes using blunt forceps. Cell-matrix constructs having eight layers were then fabricated.

The cell-matrix sheets were layered in pairs by superimposing them to yield sixteen 2-layer cell-matrix constructs. These were again allowed to air-dry in ambient room temperature and humidity in aseptic conditions. Four sets of 2-layered constructs were spread out onto a 100 mm plate and each had 20 mL of a specific type and activity of TGM or rhTGM solutions was added: (1) 8-layer food-grade TGM 5 U; (2) 8-layer food-grade TGM 10 U; (3) 8-layer rhTGM 15 U; and (4) 8-layer rhTGM 30 U. The constructs were placed into a 40° C. incubator and allowed to crosslink overnight. The following day, each condition of 2-layered constructs was layered into 4-layer constructs. This process was repeated again but without TGM to achieve one 8-layer construct for each crosslinking condition. These were air-dried in ambient room temperature and humidity and treated with the same TGM crosslinking agent for a second and final time. The constructs were placed into a 40° C. incubator and allowed to crosslink overnight, approximately 18-24 hours. After a final air-drying, the constructs were hydrated with sterile WFI. The thickness of each construct was measured using a laser. Three pieces from each 8-layered unit were tested for tensile strength and suture retention on the Instron machine.

Cross-linking with rhTGM was more successful than food-grade TGM for 8-layered constructs in this experiment. The lower concentration of rhTGM resulted in better tensile strength and suture retention than other conditions.

### Example 15

### Connective Tissue Construct

Fibroblasts were seeded retrieved from scale-up cultures where fibroblasts were cultured on microcarriers in bioreactors. Fibroblasts were strained using a stainless steel sieve set-up to separate the fibroblasts from the microcarriers. This removed all microcarriers and cell clumps from the cell suspension. Approximately 3.0×10⁷ cells were seeded to a 0.4 micron porous membrane of approximately 44 cm² in surface area bathed in about 140 mL chemically defined matrix production medium. This seeding density was at super-confluence.

The chemically defined matrix production medium contained a base of DMEM (high glucose, without L-glutamine) supplemented with approximate amounts of the following: 4 mM L-glutamine; 10 ng/ml human recombinant epidermal growth factor; 1×10⁻⁴ M ethanolamine; 1×10⁻⁴ M o-phosphoryl-ethanolamine; 5 μg/ml transferrin, 20 ρM triiodothyronine, 5 μg/ml insulin; 6.78 ng/ml selenious acid; 50 ng/ml magnesium ascorbate; 0.2 μg/ml L-proline; 0.1 μg/ml glycine; 0.02 μg/ml human recombinant long chain TGF-alpha; 0.0038 μg/ml prostaglandin E₂ (PGE2); 0.4 μg/ml hydrocortisone. Matrix production medium was exchanged with fresh matrix production medium every 3-4 days for 18 days. During this time, an endogenous cell-matrix construct had formed by the cells.

### Example 16

### Bilayer Skin Construct

A skin construct having a fibroblast layer and a keratinocyte layer was formed in a fully chemically defined culture media system. Fibroblasts were seeded retrieved from scale-up cultures where fibroblasts were cultured on microcarriers in bioreactors. Fibroblasts were strained using a stainless steel sieve set-up to separate the fibroblasts from the microcarriers. This removed all microcarriers and cell clumps from the cell suspension. Approximately 1.0×10⁷ cells were seeded to a 0.4 micron porous membrane of approximately 44 cm² in surface area bathed in about 130 mL chemically defined matrix production medium. This seeding density was at super-confluence.

The chemically defined matrix production medium contained:

| Component | Concentration | |
|---|---|---|
| DMEM | 96.0% | |
| L-Glutamine | 1060 | mg/L |
| Hydrocortisone | 0.4 | mg/L |
| Selenious acid | 6.78 | μg/L |
| Ethanolamine | 0.1 | mM |
| o-Phosphorylethanolamine | 14.0 | Mg/L |
| EGF | 10.0 | μg/L |
| Mg Ascorbate | 50 | mg/L |
| L-Proline | 213.6 | mg/L |
| Glycine | 101.4 | mg/L |
| Long TGFα | 10.0 | μg/L |

Fibroblasts were cultured in the matrix production medium for 11 days with media changes made periodically, every 3-4 days.

At day 11, a suspension of keratinocytes was seeded onto the surface of the cell-matrix construct at an approximate density of 3.3×10⁶ cells in a medium containing approximately:

| Component | Concentration | |
|---|---|---|
| DMEM:HAM's F-12 3:1 | 96.10% | |
| L-Glutamine | 1060 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| o-Phosphorylethanolamine | 14.0 | Mg/L |
| Selenious acid | 6.78 | μg/L |
| Adenine | 24.4 | mg/L |
| Mg Ascorbate | 50.0 | mg/L |
| Progesterone | 0.63 | μg/L |
| EGF | 10.0 | μg/L |
| Long TGFα | 10.0 | μg/L |
| Lipid Concentrate | Arachidonic Acid | 0.004 mg/L |
| | Cholesterol | 0.220 mg/L |
| | DL-α-Tocopherol- | 0.140 mg/L |
| | Acetate | |
| | Linoleic Acid | 0.020 mg/L |
| | Linolenic Acid | 0.020 mg/L |
| | Myristic Acid | 0.020 mg/L |
| | Oleic Acid | 0.020 mg/L |
| | Palmitoleic Acid | 0.020 mg/L |
| | Palmitic Acid | 0.020 mg/L |
| | Pluronic ® F-68 | 200.0 mg/L |
| | Stearic Acid | 0.020 mg/L |
| | Tween ® 80 | 4.4 mg/L |

At day 13, differentiation was induced by adding use of a differentiation medium containing the following:

| Component | Concentration | |
|---|---|---|
| DMEM:HAM's F-12 3:1 | 96.3% | |
| L-Glutamine | 1060 | mg/L |
| Hydrocortisone | 0.40 | mg/L |
| Insulin | 5.0 | mg/L |
| Transferrin | 5.0 | mg/L |
| Triiodothyronine | 13.5 | ng/L |
| Selenious acid | 0.00678 | mg/L |
| Ethanolamine | 0.1 | mM |
| o-Phosphorylethanolamine | 14.0 | Mg/L |
| Adenine | 24.4 | mg/L |
| Mg Ascorbate | 50.0 | mg/L |
| Progesterone | 0.63 | μg/L |
| CaCl2 | 265 | mg/L |
| Lipid Concentrate | Arachidonic Acid | 0.004 mg/L |
| | Cholesterol | 0.220 mg/L |
| | DL-α-Tocopherol- | 0.140 mg/L |
| | Acetate | |
| | Linoleic Acid | 0.020 mg/L |
| | Linolenic Acid | 0.020 mg/L |
| | Myristic Acid | 0.020 mg/L |
| | Oleic Acid | 0.020 mg/L |
| | Palmitoleic Acid | 0.020 mg/L |
| | Palmitic Acid | 0.020 mg/L |
| | Pluronic ® F-68 | 200.0 mg/L |
| | Stearic Acid | 0.020 mg/L |
| | Tween ® 80 | 4.4 mg/L |

At day 15, the medium formulation was changed to induce cornification of the developing keratinocyte layer in a medium containing approximately:

| Component | Concentration | |
|---|---|---|
| DMEM | 48.0% | |
| HAM's F-12 | 48.0% | |
| L-Glutamine | 658 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| o-Phosphorylethanolamine | 14.0 | Mg/L |
| Selenius acid | 6.78 | μg/L |
| Adenine | 24.4 | mg/L |
| Mg Ascorbate | 50.0 | mg/L |
| Long TGFα | 10.0 | μg/L |

| | | | |
|---|---|---|---|
| MEM Non-Essential Amino Acid | L-Alanine | 1.78 | mg/L |
| Solution | L-Asparagine | 2.64 | mg/L |
| | L-Aspartic Acid | 2.66 | mg/L |
| | L-Glutamic Acid | 2.94 | mg/L |
| | Glycine | 1.5 | mg/L |
| | L-Proline | 2.3 | mg/L |
| | L-Serine | 2.1 | mg/L |
| MEM Vitamin Solution | NaCl | 17 | mg/L |
| | D-Ca | 0.2 | mg/L |
| | Pantothenate | | |
| | Choline | 0.2 | mg/L |
| | Chloride | | |
| | Folic Acid | 0.2 | mg/L |
| | i-Inositol | 0.4 | mg/L |
| | Nicotinamide | 0.2 | mg/L |
| | Pyridoxal HCl | 0.2 | mg/L |
| | Riboflavin | 0.020 | mg/L |
| | Thiamine HCl | 0.2 | mg/L |
| Lipid Concentrate | Arachidonic | 0.004 | mg/L |
| | Acid | | |
| | Cholesterol | 0.220 | mg/L |
| | DL-α- | 0.140 | mg/L |
| | Tocopherol- | | |
| | Acetate | | |
| | Linoleic Acid | 0.020 | mg/L |
| | Linolenic Acid | 0.020 | mg/L |
| | Myristic Acid | 0.020 | mg/L |
| | Oleic Acid | 0.020 | mg/L |
| | Palmitoleic Acid | 0.020 | mg/L |
| | Palmitic Acid | 0.020 | mg/L |
| | Pluronic ® F-68 | 200.0 | mg/L |
| | Stearic Acid | 0.020 | mg/L |
| | Tween ® 80 | 4.4 | mg/L |

Cornfication medium was changed every 2-3 days.

Skin constructs matured and maintained during days 22 through 35 and were fed a maintenance medium with changes every 2-3 days with fresh maintenance medium containing:

| Component | Concentration | |
|---|---|---|
| DMEM | 48.0% | |
| HAM's F-12 | 48.0% | |
| L-Glutamine | 658 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| O-phosphorylethanolamine | 14.0 | Mg/L |
| Selenius acid | 6.78 | μg/L |
| Adenine | 24.4 | mg/L |
| Long TGFα | 10.0 | μg/L |

| | | | |
|---|---|---|---|
| MEM Non-Essential Amino Acid | L-Alanine | 1.78 | mg/L |
| Solution | L-Asparagine | 2.64 | mg/L |
| | L-Aspartic Acid | 2.66 | mg/L |
| | L-Glutamic Acid | 2.94 | mg/L |
| | Glycine | 1.5 | mg/L |
| | L-Proline | 2.3 | mg/L |
| | L-Serine | 2.1 | mg/L |
| MEM Vitamin Solution | NaCl | 17 | mg/L |
| | D-Ca | 0.2 | mg/L |
| | Pantothenate | | |
| | Choline | 0.2 | mg/L |
| | Chloride | | |
| | Folic Acid | 0.2 | mg/L |
| | i-Inositol | 0.4 | mg/L |
| | Nicotinamide | 0.2 | mg/L |
| | Pyridoxal HCl | 0.2 | mg/L |
| | Riboflavin | 0.020 | mg/L |
| | Thiamine HCl | 0.2 | mg/L |
| Lipid Concentrate | Arachidonic | 0.004 | mg/L |
| | Acid | | |
| | Cholesterol | 0.220 | mg/L |
| | DL-α- | 0.140 | mg/L |
| | Tocopherol- | | |
| | Acetate | | |
| | Linoleic Acid | 0.020 | mg/L |
| | Linolenic Acid | 0.020 | mg/L |
| | Myristic Acid | 0.020 | mg/L |
| | Oleic Acid | 0.020 | mg/L |
| | Palmitoleic Acid | 0.020 | mg/L |
| | Palmitic Acid | 0.020 | mg/L |
| | Pluronic ® F-68 | 200.0 | mg/L |
| | Stearic Acid | 0.020 | mg/L |
| | Tween ® 80 | 4.4 | mg/L |

When fully formed the cultured skin constructs exhibited a cell-matrix layer of endogenously produced extracellular matrix and its fibroblasts with a differentiated keratinocyte layer disposed atop the cell-matrix layer.

### Example 17

### Fabrication of Collagenous Material-Synthetic Polymer Constructs Using Silk Fibroin Adhesive Solution

Adhesion properties of a purified xenogeneic collagen layer (e.g. “ICL”) with various objects were tested. More particularly, without having the intention of being limited to the following embodiments, adhesion properties between (a) two ICL layers; (b) at least one ICL layer and at least one polyhydroxyalkanoates-(PHA)-based polymeric frame; and (c) at least one ICL layer to at least one PHA-based polymeric rod were analyzed. Preparation of the samples are described in the following examples:
(a) A dried single layer ICL was cut into pieces of 1×2.5 cm in size. Two pieces of ICL was attached with an overlapping size of 0.5 cm and a drop of silk solution was applied between the layers. It is to be appreciated that the dimensions of the cut ICL pieces, and the overlapping size, are not limited to the above examples.
(b) A PHA polymeric-based frame was dipped in silk fibroin solution for at least one minute to coat the frame surface struts with a thin layer of the silk fibroin solution. A piece of ICL (about 1.5×1.5 cm) was then attached to the leading end of the frame.
(c) A PHA polymeric-based rod was dipped in silk fibroin solution for at least one minute in an amount sufficient to coat the surface of the rod with a thin layer of silk fibroin solution. A piece of ICL (about 1×1.5 cm) was then attached to the rod.

Subsequently, all the samples were dried at room temperature for about 1.5 hours. The samples were then immersed in a methanol-based solvent for 5 to 10 minutes, and subsequently dried for 10 minutes. The above samples were soaked in deionized water for at least 24 hours. Adhesion properties achieved by the silk fibroin solution are then evaluated by introducing mechanical strain to the samples using procedures known to one of ordinary skill in the art.

Without having the intention of being limited, it is to be appreciated that employing the silk fibroin aqueous solution as an adhesive can be implemented, for example, between ICL and electrospun collagen, electrospun fibrin, metallic-based materials, ceramic-based materials, tissue-engineered constructs, synthetic polymer materials, natural materials.

### Example 18

### Fabrication of Multi-Layer Cell-Matrix Constructs Using Silk Fibroin Adhesive Solution

In order to make multi-layer cell-tissue constructs, units were first removed from culture, growth and/or matrix production media were aspirated, and the units were allowed to air dry at least until the cells were devitalized. The units were then rehydrated in 10 ml of WFI for about 10 minutes. Additionally and/or alternatively, the units were rehydrated in 5 mL or exposed to about 0.5 mL of about an 8% silk solution, and the units were layered together. The units are then dried overnight, and then subsequently dipped in 3 mL of methanol for at least 10 minutes. The above steps can be repeated until 5 or more layer cell-tissue constructs are formed. As an alternative, it is to be appreciated that transglutaminase can be applied between the layers of the multi-layer cell tissue-constructs. Additionally, after the step of treating the layers with the 8% silk solution, the units can be rolled flat using a roller.

The 5 or more layer cell-tissue constructs can subsequently be lyophilized by placing the constructs in a lyophilizer for at least about 17 hours. It is to be appreciated that the silk solution serves as an adhesive to prevent the layered construct from delamination.

### Example 19

### Bioengineered Construct Produced by Mesenchymal Stem Cells (MSCs)

Generation of bioengineered constructs comprising mesenchymal stem cells grown under conditions to produce a layer of extracellular matrix which is synthesized and assembled by the mesenchymal stem cells is exemplified using human umbilical cord perivascular cells (HUCPVC). Specifically, skilled artisans have heretofore been unable to define preparatory conditions for allowing MSCs to synthesize and assemble extracellular matrix components to any appreciable thickness. Prior to seeding the HUCPVC, culture inserts were coated with about 5 ug/cm² of human plasma-derived fibronectin. The bioengineered constructs were produced by initially seeding 3×10⁶ HUCPVC per 24 mm insert. Subsequent to seeding the cells upon a culture insert with a porous membrane in a insert, the cells were maintained in culture for 18 days, with replacement with fresh culture media at days 5, 8, 12, and 15, in the following chemically defined culture medium:

| Component | Concentration (Volume for 1 L) | |
|---|---|---|
| DMEM | 96.0% (960 mL in 1 L) | |
| L-Glutamine | 1060 | mg/L |
| Hydrocortisone | 0.4 | mg/L |
| Selenious acid | 6.78 | μg/L |
| ITT (2.5 mg/mL Insulin + 2.5 mg/mL | 2 | mL |
| Transferrin + 6.74 ng/mL | | |
| Triiodothyronine) | | |
| EOP ((3.103 g/L monoethalonamine + | 2 | mL |
| 7.06 g/L o-Phosphorylethanolamine) | | |
| EGF | 10.0 | μg/L |
| Mg Ascorbate | 50 | mg/L |
| L-Proline | 213.6 | mg/L |
| Glycine | 101.4 | mg/L |
| Long TGFα (Novozymes A/S) | 200 | ng/mL |
| Prostaglandin 2 | 0.038 | μg/mL |

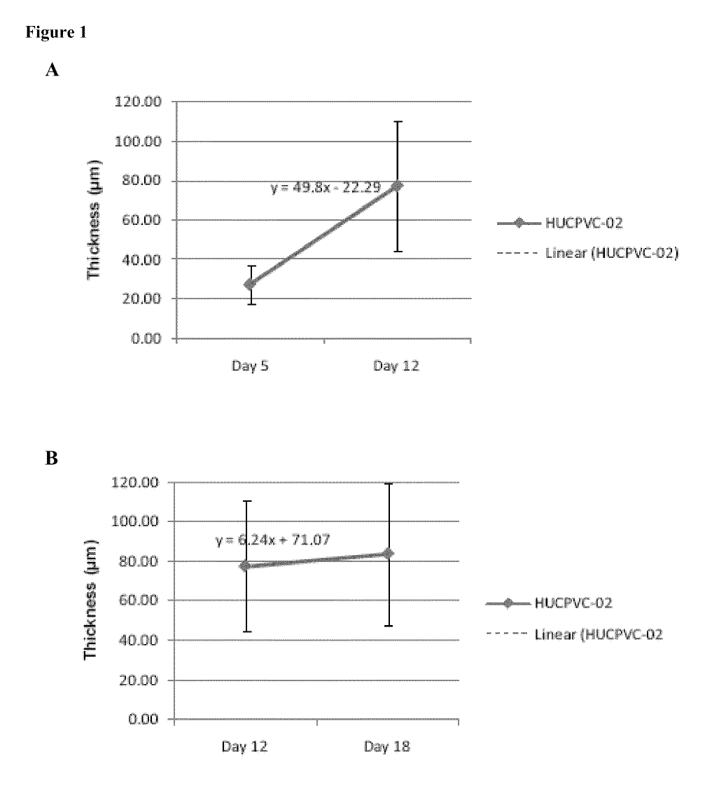

The resulting bioengineered constructs generate extracellular matrices that are at least 30 microns thick. Time-course analysis of extracellular matrix formation was conducted to correlate MSC-derived bioengineered construct thickness with lengths of culture time. FIGS. 1A and 1B demonstrate that the greatest increases in bioengineered construct thickness can be achieved by twelve days of culturing.

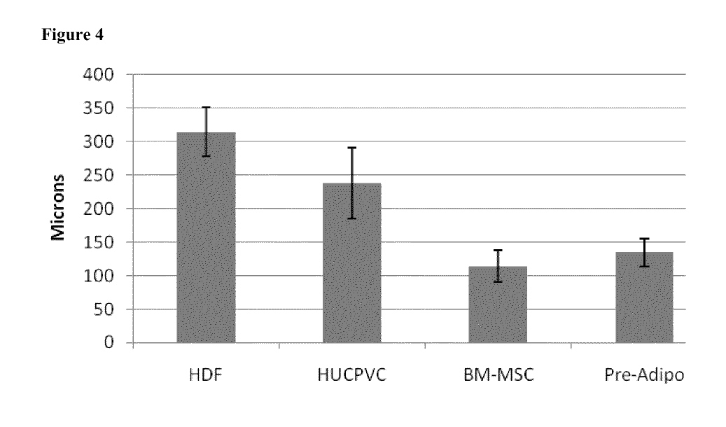

In order to further define the factors contributing to efficient extracellular matrix synthesis and assembly by mesenchymal stem cells, the role of TGF-alpha and prostaglandin 2 were assessed. FIG. 2 demonstrates the correlation between increasing bioengineered construct thickness as a function of increased TGF-alpha concentration in the culture media after culturing 3×10⁶ HUCPVC per 24 mm insert for 18 days. FIG. 3 demonstrates the correlation between decreasing bioengineered construct thickness as a function of increased prostaglandin 2 concentration in the culture media after culturing 3×10⁶ HUCPVC per 24 mm insert for 18 days. Accordingly, the amount of extracellular matrix synthesized and assembled by mesenchymal stem cells can be modulated based on culture media components and, in particular, appreciable thicknesses of the resulting bioengineered construct can be achieved. In addition, culture media supplementation can synergize with increased seeding densities (such as superconfluent densities containing 3×10⁶ to 10×10⁶ cells or more per 24 mm insert) to produce even thicker extracellular matrices in MSC-derived bioengineered constructs, including those derived from HUCPVC, bone marrow-derived MSCs, and pre-adipocytes (FIG. 4). In a specific embodiment, superconfluent cell seeding was performed using 30×10⁶ cells per 75 mm insert, which is equivalent to 9.6×10⁶ cells per 24 mm insert.

### Example 20

### Biophysical Properties of Bioengineered Constructs Produced by Mesenchymal Stem Cells (MSCs)

In addition to generating appreciable amounts of synthesized and assembled extracellular matrix by mesenchymal stem cells to produce bioengineered construct having significant thicknesses, such bioengineered constructs have additional biophysical properties that distinguish them from extracellular matrices formed by other cell types.

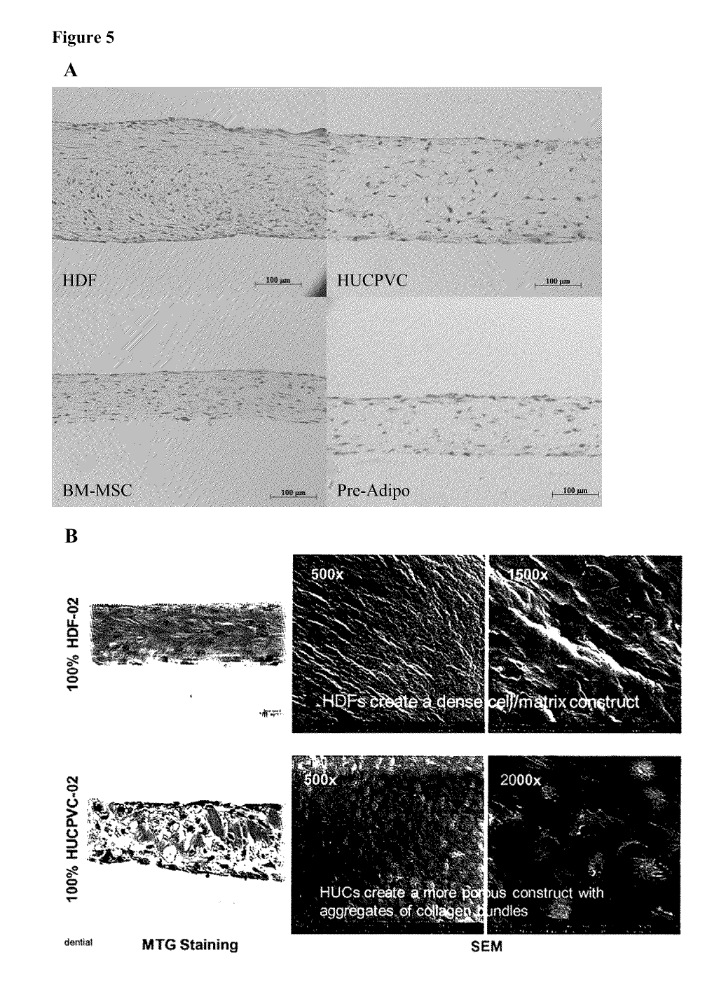

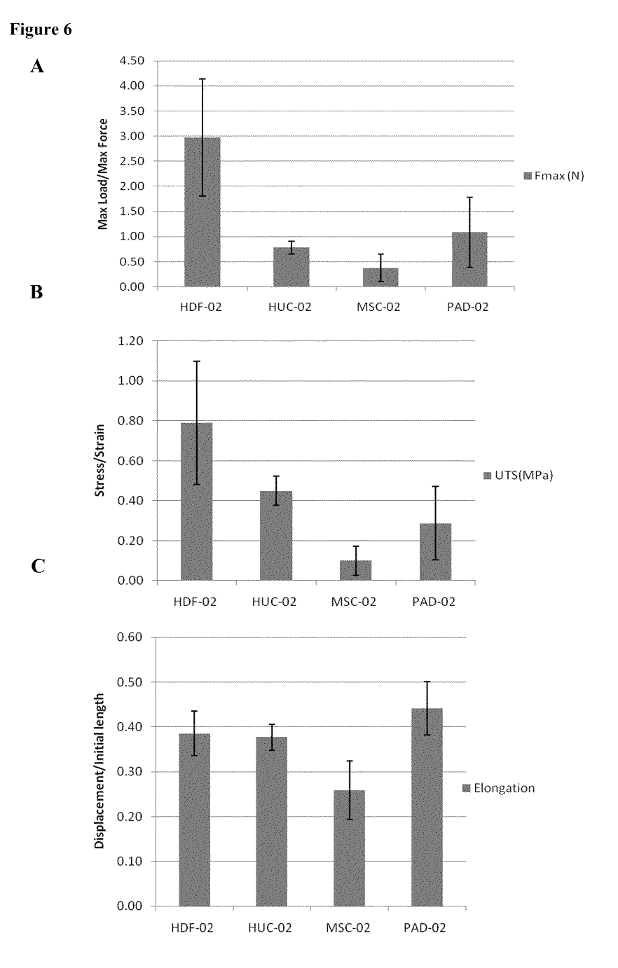

MSC-derived bioengineered constructs seeded at superconfluency and cultured for 18 days according to the methods and culture media defined in Example 19 exhibited a significant difference in collagen arrangement and overall matrix morphology from similarly cultured HDF-derived bioengineered constructs (except using 20 ng/mL TGF-alpha). In particular, the extracellular matrix containes pore, is less dense, and contains aggregates of collagen bundles (FIGS. 5A-5B). Thus, MSC-derived bioengineered constructs have a porosity, which can be represented as the percentage area that is represented by pores in a histological section relative to the total area of the histological section. Such a porous extracellular matrix is desirable for many wound healing indications since it allows for greater migration and infiltration of host cells and angiogenesis-related molecules once grafted into a wound. However, such porous extracellular matrices should also maintain mechanical integrity to allow a physician to apply the bioengineered construct with minimal difficulty. Accordingly, mechanical testing of the MSC-derived and HDF-derived bioengineered constructs were performed to evaluate several mechanical properties. Specifically, Fmax (also known as Max load/Max force, e.g. 1, 2, 3, 4, 5, 6, 7, 8, 9, 10 N) is the maximum load that can be applied onto a material before it breaks. Ultimate tensile strength (also known as UTS, e.g. 5, 10, 15, 20, 25, 30, 35, 40, 45, 50 N/cm²) is the maximum pressure load sustained by a specimen prior to rupture. Elasticity modulus (also known as elongation, e.g. 0.1, 0.2, 0.3, 0.4, 0.5, 0.6, 0.7, 0.8, 0.9 Displacement/Initial length) is a measure of the stiffness of a material within the linear region whereby the material will return to a starting condition if the load is removed. FIGS. 6A-6C show that MSC-derived bioengineered constructs have similar mechanical integrity to HDF-derived bioengineered constructs despite having a more porous extracellular matrix, with HUCPVC-derived bioengineered constructs having the most similar mechanical integrity and thickness profile.

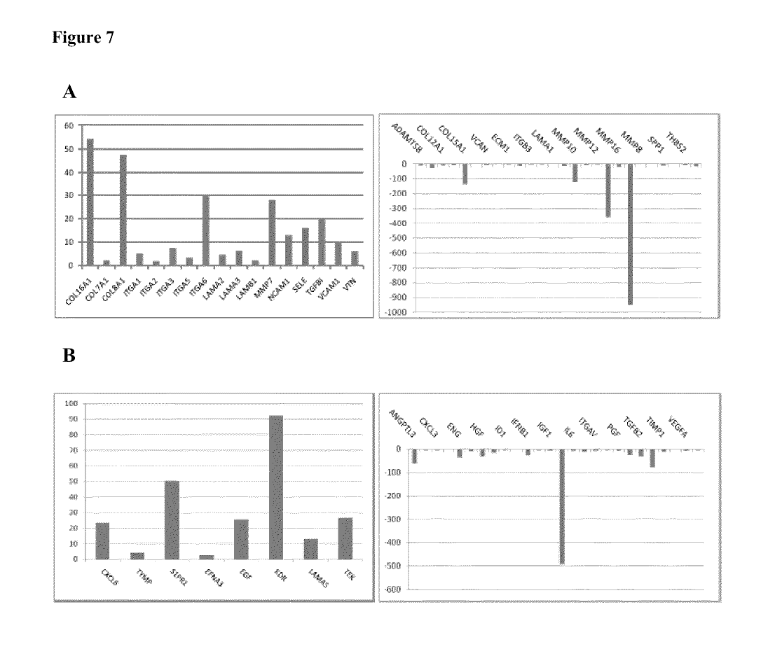

A bioengineered construct having a porous extracellular matrix with strong mechanical properties would be further useful for treating wounds by allowing the diffusion of growth factors at the delivery site that promote wound healing. In order to characterize differences in extracellular matrix components, adhesion components, and/or growth factors present between the MSC-derived bioengineered constructs and those generated using other cell types, quantitative PCR (qPCR) assays were performed using cDNA isolated from MSC-derived bioengineered constructs seeded at superconfluency and cultured for 18 days (according to the methods and culture media defined in Example 19) or human dermal fibroblast (HDF)-derived bioengineered constructs seeded at superconfluency and cultured for 18 days (according to the methods and culture media defined in Example 19, except that the culture media was supplemented with 20 ng/mL Long TGFα). Real-time PCR primers from the Human ECM and Adhesion Molecules Array (SuperArray PAHS-013A) and the Human Growth Factor Array (SuperArray PAHS-041A) according to the manufacturer's protocol. FIG. 7 shows a summary of differences in growth factors between MSC-derived and HDF-derived bioengineered constructs. For example, the increased collagen expression in HUCPVC-derived bioengineered constructs is consistent with the collagen bundling characteristics observed in FIG. 5. Increased expression of CXCL6, a chemoattractive molecule; KDR, an indicator of VEGF-induced proliferation, migration, tubular morphogenesis, and endothelial sprouting; and laminin alpha 5 (LAMAS), an indicator of embryonic cell organization, was also observed in HUCPVC-derived bioengineered constructs. These results demonstrate that, in addition to the appreciable thicknesses of extracellular matrix achieved using MSC-derived bioengineered constructs, such constructs also exhibit upregulation of genes useful in treating a wound environment, such as promoting healing rates and angiogenesis (FIG. 7).

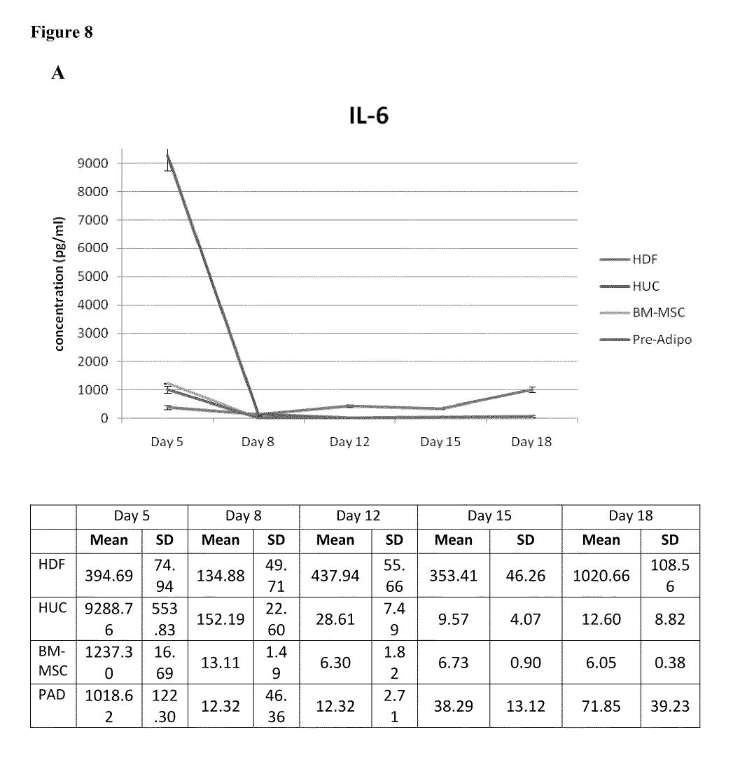

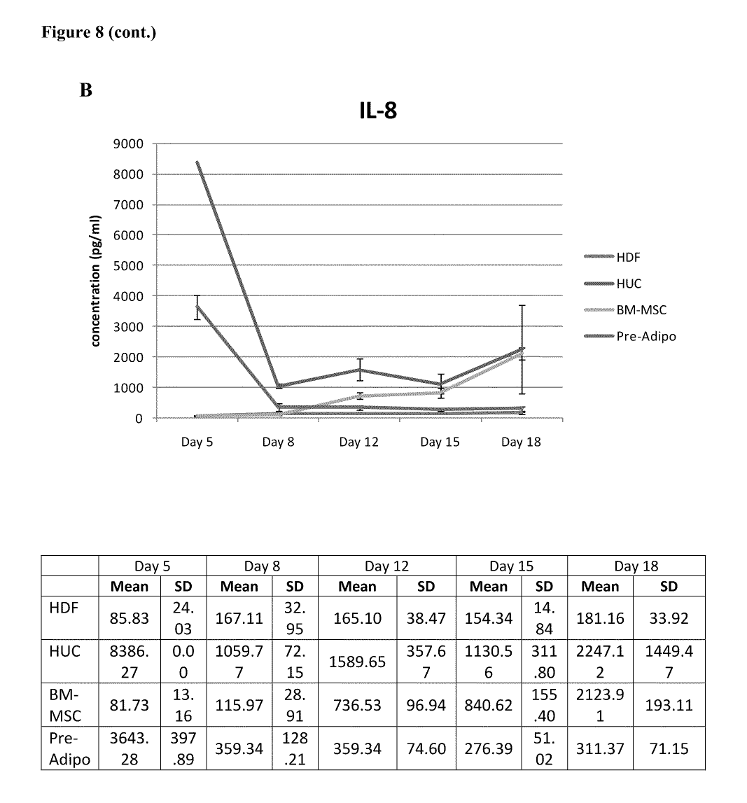

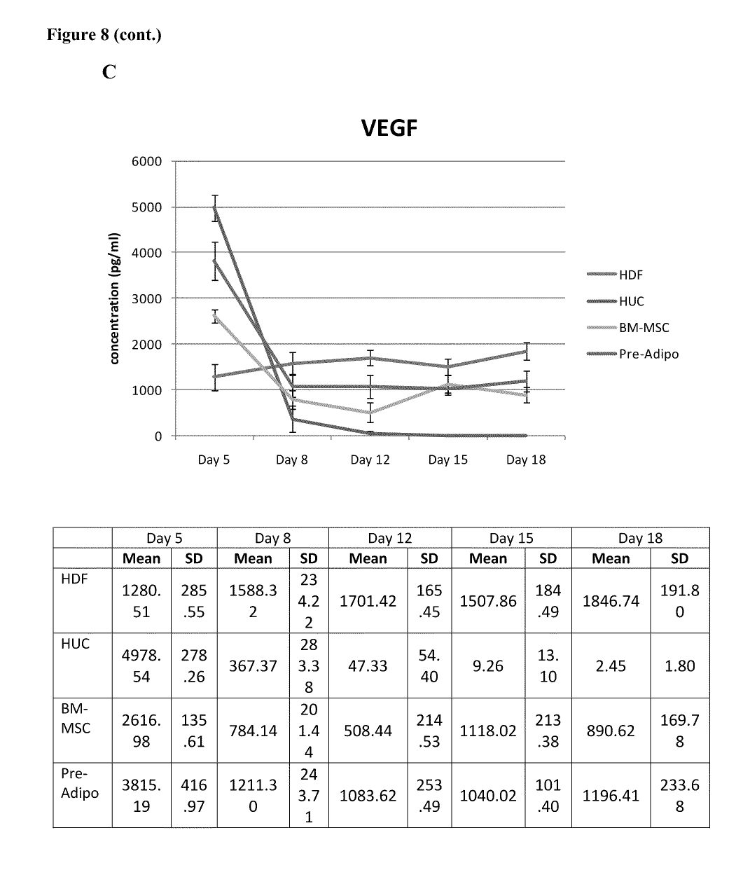

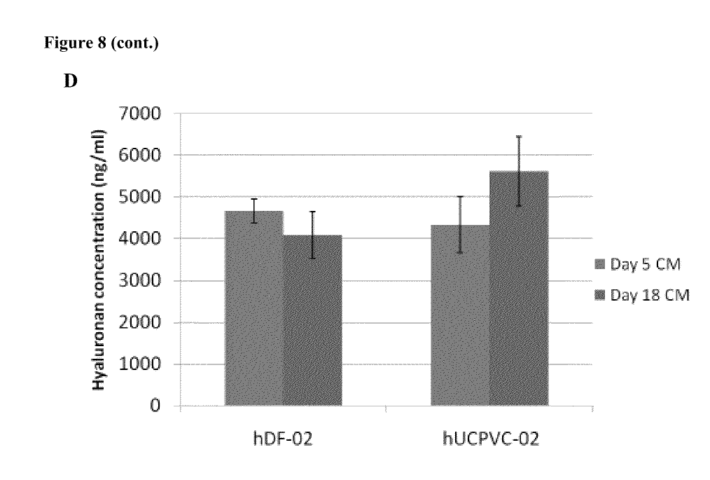

In addition, protein-based assays for detecting IL-6, IL-8, and VEGF levels using the cytometric bead array system (CBA) from Becton Dickinson were performed using MSC-derived bioengineered constructs seeded at superconfluency and cultured for 18 days (according to the methods and culture media defined in Example 19) or human dermal fibroblast (HDF)-derived bioengineered constructs seeded at superconfluency and cultured for 18 days (according to the methods and culture media defined in Example 19, except that the culture media was supplemented with 20 ng/mL Long TGFα), according to the manufacturer's protocol. FIGS. 8A-8C show a time-course comparison of IL-6, IL-8, and VEGF levels within the conditioned media generated by the MSC-derived and HDF-derived bioengineered constructs. IL-6 expression in MSC-derived bioengineered constructs peaked early during the culture time-course and was over 9 times that of the HDF-derived bioengineered constructs at day 5 of culturing HUCPVC-derived bioengineered constructs (FIG. 8A). Besides its role in the immune response, IL-6 is also secreted by osteoblasts to promote osteoclast formation. IL-8 expression was also significantly overexpressed in MSC-derived bioengineered constructs relative to HDF-derived bioengineered constructs over the entire length of culture (FIG. 8B). Besides its role in the immune response, IL-8 is also secreted by epithelial cells as a potent angiogenic factor since via binding to such receptors as CXCR1 and CXCR2. Similarly, VEGF is another potent angiogenic factor and is significantly overexpressed in MSC-derived bioengineered constructs relative to HDF-derived bioengineered constructs during the early phases of culture (FIG. 8C). It is believed that the drop in detectable VEGF levels in the culture media are due to the high levels of KDR expression by HUCPVCs and other MSCs, which is the receptor for VEGF and sequesters the molecules within the bioengineered construct so as to preclude detection in the media. In addition, CSF-3 and vitronectin are upregulated in HUCPVC-derived bioengineered constructs relative to HDF-derived bioengineered constructs. An ELISA assay was further performed on conditioned media samples of culturing HDF-derived and MSC-derived bioengineered constructs according to the methods of Example 19 (i.e., 10× TGF-alpha for both conditions) to quantify the amount hyaluronan (HA) production after 5 and 18 days. FIG. 8D shows that whereas HA levels in culture media of HDF-derived bioengineered constructs decreased from 4,664 ng/mL on day 5 to 4,085 ng.mL on day 18, HA levels in culture media of HUCPVC-derived bioengineered constructs increased from 4,333 ng/Ml on day 5 to 5,615 ng/mL on day 18. In addition, MSC-derived bioengineered constructs exhibited 38-fold more vitronectin, 21-fold more CSF-3, 15-fold more NCAM1, and 4-fold more CXCL1 relative to HDF-derived bioengineered constructs.

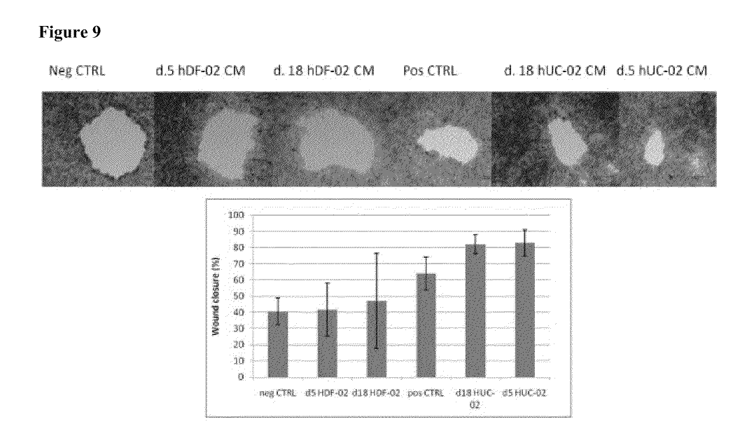

Finally, MSC-derived bioengineered constructs seeded at superconfluency and cultured for 18 days according to the methods and culture media defined in Example 19, yielded a conditioned medium having components that increase the ability for cells to migrate relative to HDF-derived bioengineered constructs cultured under identical conditions, except that the culture media was supplemented with 20 ng/mL Long TGFα (FIG. 9).

### Example 21

### Multilineage Potential Properties of Bioengineered Constructs Produced by Mesenchymal Stem Cells (MSCs)

Assays were performed to determine the multilineage potential properties of cells isolated from bioengineered constructs produced by MSCs, as well as from MSCs within the native bioengineered construct environment. MSC-derived bioengineered constructs were seeded at superconfluency and cultured for 18 days according to the methods and culture media defined in Example 19. At day 18, the bioengineered constructs were either digested with collagenase to determine cell yields and cell digests for multilineage potential assays or directly cultured in induction media. Non-induced MSC control groups of cells and bioengineered constructs were maintained for each of the induced cell and bioengineered construct groups, wherein alpha MEM media supplemented with 10% fetal bovine serum (FBS) was used in the place of induction media. Media changes occurred every 2-3 days. In addition, HDF-derived control groups of cells and bioengineered constructs were maintained for each of the induced cell and bioengineered construct groups.

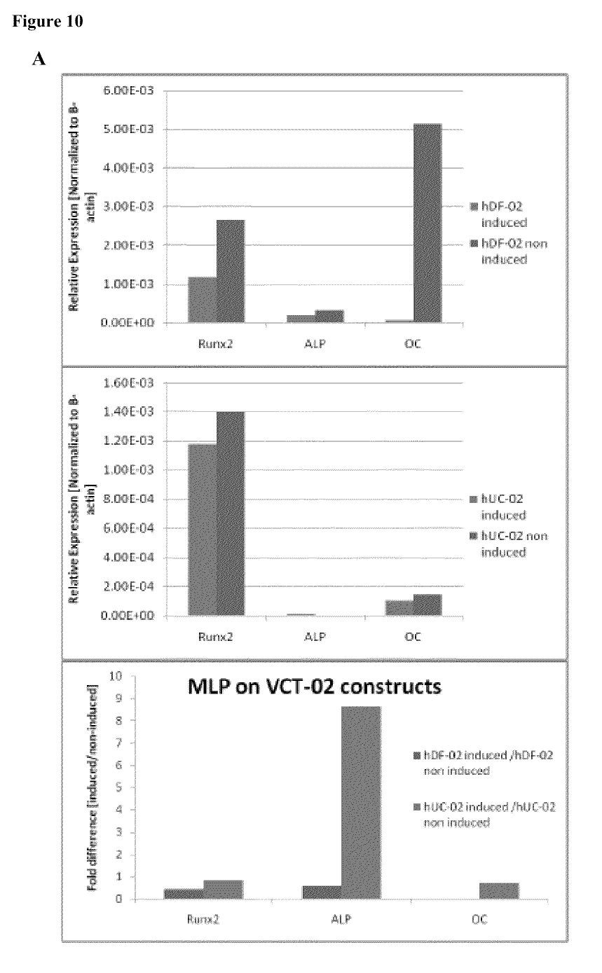

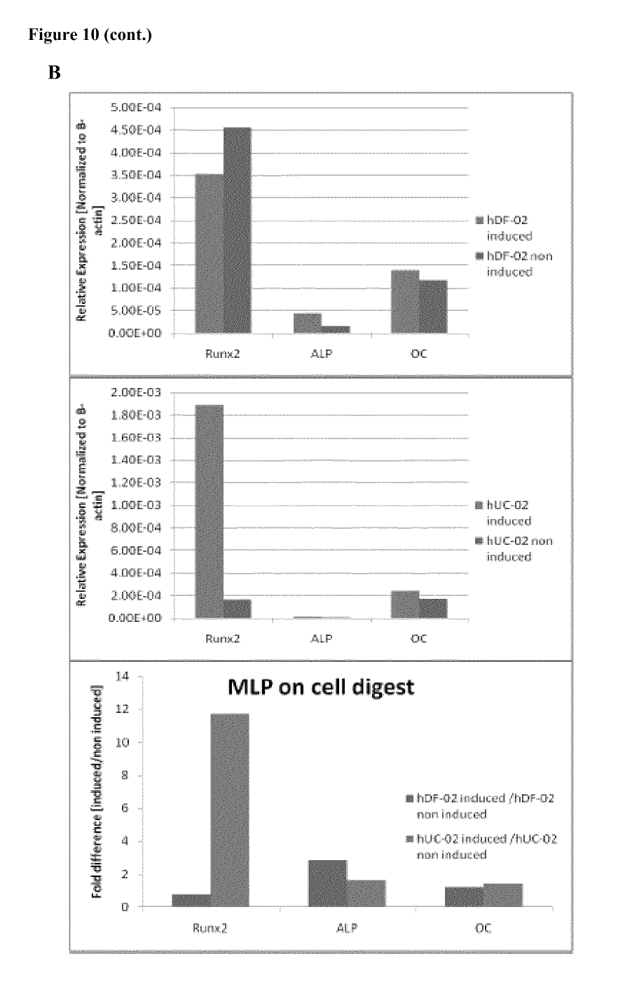

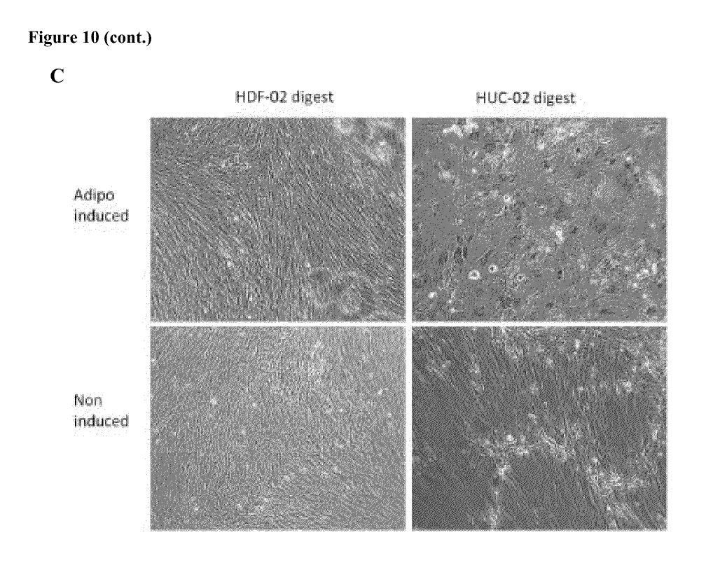

For the osteogenic induction assay, bioengineered constructs were directly cultured in osteogenic induction media and cells resulting from collagenase digestion were seeded at 20,000 cells/cm² in 12-well plates for osteogenic induction. The defined culture media shown in Example 19 was replaced at day 18 of culture with the following osteogenic induction media: Complete DMEM base media supplemented with 10⁻³ M dexamethasone (DEX), 1M β-glycerophosphate (BGP), and 50 mg/mL ascorbic acid (AA). Osteogenic induction culturing occurred for days prior to analysis of gene expression of Runx2 (a transcription factor expressed in the later stages of osteoblastic differentiation), ALP, and osteoclacin (OC) using RNA isolated from the bioengineered constructs or cultured cells. An 8-fold increase in the expression of ALP was observed in the induced MSC-derived bioengineered construct relative to non-induced MSC-derived bioengineered constructs (FIG. 10A). In addition, an 11-fold increase in the expression of Runx2 was observed in isolated MSC-derived bioengineered construct cells that were induced in osteogenic induction media relative to such cells that were not induced in osteogenic induction media (FIG. 10B). Thus, MSCs within an intact bioengineered construct or isolated from such constructs can be induced toward an osteogenic lineage based on environmental signaling cues.

For the adipogenic induction assay, bioengineered constructs were directly cultured in adipogenic induction media and cells resulting from collagenase digestion were seeded at 20,000 cells/cm² in 12-well plates for adipogenic induction. The defined culture media shown in Example 19 was replaced at day 18 of culture with the following adipogenic induction media: Complete DMEM base media supplemented with 10⁻³ M dexamethasone (DEX), 10 mg/mL insulin, and 0.5 mM 3-isobutyl-1-methylxanthine (IBMX). Osteogenic induction culturing occurred for days prior to analysis of neutral triglycerides and lipids from the bioengineered constructs or cultured cells using standard Oil Red-0 staining Only isolated MSC-derived bioengineered construct cells that were induced in adipogenic induction media were observed to have a significant number of positively stained cells relative to such cells that were not induced in adipogenic induction media (FIG. 10C). Thus, MSCs within an intact bioengineered construct or isolated from such constructs can be induced toward an adipogenic lineage based on environmental signaling cues.

Thus, MSCs within and isolated from an intact bioengineered construct can be induced toward several cell lineages based on environmental signaling cues while a sub-population is maintained with stem-like potential.

### Example 22

### In vivo Vascularization Properties of Bioengineered Constructs Produced by Mesenchymal Stem Cells (MSCs)

The purpose of this study was to graft bioengineered constructs produced by the methods of Example 19 onto nude mice, and analyze their response in vivo when implanted subcutaneously. More particularly, alpha-Smooth Muscle Actin (αSMA) staining was used to qualitatively and quantitatively analyze vascularization within the construct in the mice. Units were grafted in a subcutaneous implantation model in female Swiss nude mice at 8 weeks old.

After 1 week following subcutaneous implantation of the various bioengineered constructs, 5 animals from each group listed in the following table were sacrificed:

| | | | # of |
|---|---|---|---|
| | Time- | | units per |
| Groups | points | Total # units/readouts | groups |
| A: 100% MSC-derived | 1 week | 3 × 24 mm for Histology | 5 plates/ |
| bioengineered construct | 4 weeks | 3 × 24 mm for qPCR | group |
| B: 50% admixed | | (stem cell panel) | (30 units) |
| bioengineered construct | | 6 × 24 mm for CFU-F | |
| C: 10% admixed | | and FC (post-digesting) | |
| bioengineered construct | | 10 × 14 mm punch for | |
| D: (control) 100% HDF- | | grafting (but round up | |
| derived bioengineered | | to 3 plates/group) | |
| construct | | | |

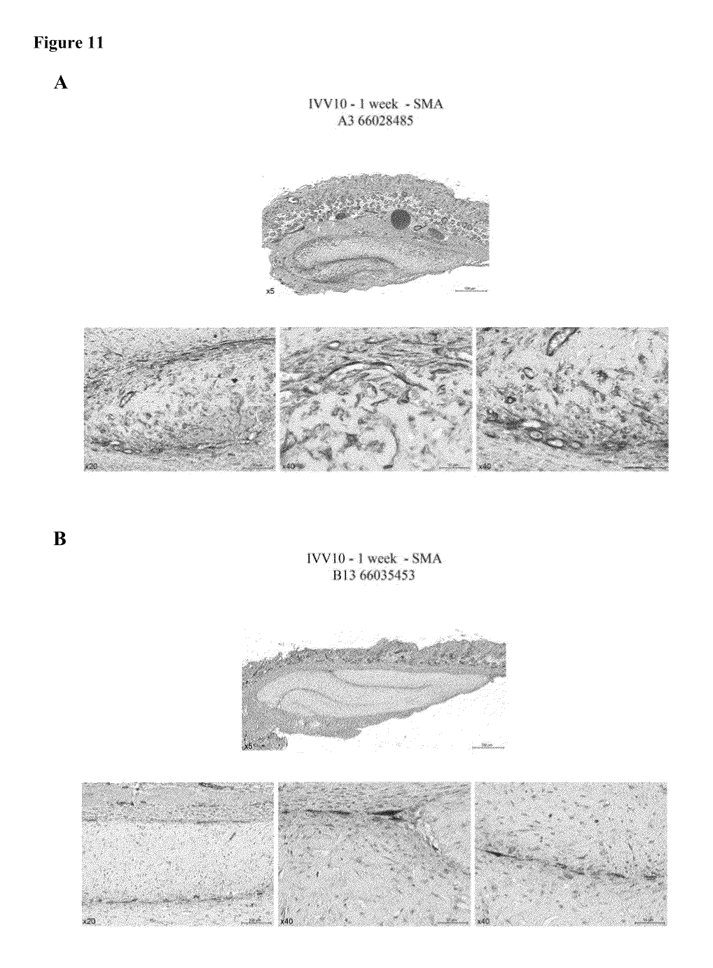

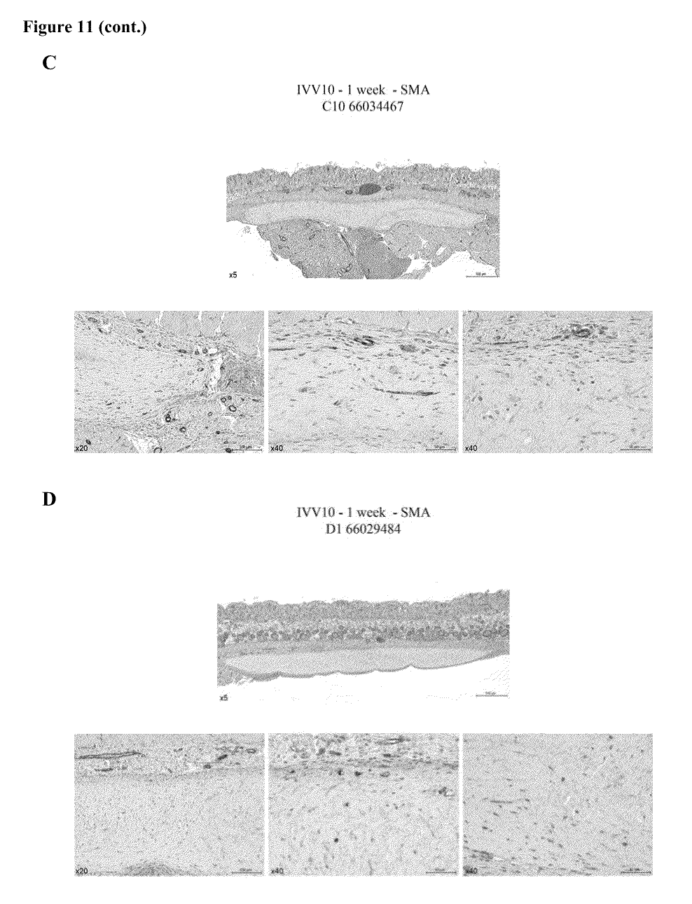

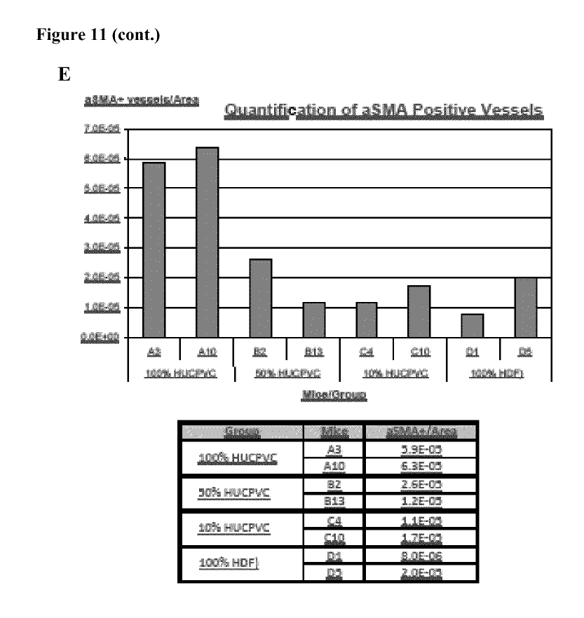

The implant area was removed and processed for histological examination. In particular, histological sections from n=2 animals from each group were stained with αSMA. FIGS. 11A-11D show representative sections taken of αSMA stained sections from 100% HUCPVC-derived bioengineered constructs, 50% HUCPVC-50% HDF-derived bioengineered constructs, 10% HUCPVC-90% HDF-derived bioengineered constructs, and 100% HDF-derived bioengineered constructs, respectively. All bioengineered constructs were produced as described in Example 19, with the exception of 100% HDF-derived constructs were cultured with 20 ng/mL TGF-alpha. The bioengineered constructs in FIG. 11A appear to have a more pronounced number of αSMA positive staining within the implant area as compared to the constructs of FIGS. 11B-11D. The αSMA staining is specifically associated around newly formed vessels, which is clearly seen in FIG. 11A at 40× magnification. Quantification of αSMA revealed that the 100% HUCPVC produced bioengineered constructs had greater numbers of vessels within the implant area related to the other groups (FIG. 11D). While not wishing to be bound by theory, the HUCPVC can secrete cytokines/growth factors, such as those describe above in Examples 20 and 21 that act in a paracrine fashion to recruit mouse endothelial cells, which then subsequently form new vessels. In addition, the matrix and its associated organization that is generated by the HUCPVC can provide a more suitable provisional matrix for cell recruitment and infiltration into the implant area, leading to the higher vessel formation seen at 1 week relative to other groups. Additionally, standard angiogenesis assays can be performed to further confirm the increased ability of HUCPVC-derived bioengineered constructs to promote angiogenesis, such as assaying for the ability of constructs to form and/or maintain tubule from endothelial cells (e.g., an angiogenesis tube formation assay from Millipore) and gene expression analyses of angiogenesis biomarkers (e.g., angiogenesis ELISA assays from Q-Plex and, angiogenesis proteome profiler array assays from R&D Systems).

### Example 23

### Controlling Contraction of Bioengineered Constructs

Bioengineered constructs were produced by seeding human neonatal foreskin fibroblasts onto 75 mm membrane inserts with plasma-treated (COOH) PES membranes that comprise 5 micron pores. The initial cell-seeding density was 30 million cells per membrane insert. Cells were suspended in chemically defined culture media (containing no undefined non-human components) with 20 ml of suspension seeded directly onto the insert, and 110 ml of media in the culture reservoir to allow for bilateral feeding of the cells. The medium contained: a base 3:1 mixture of DMEM, 2 mM L-Glutamine (Invitrogen Inc.)4 mM GlutaMAX (Gibco BRL, grand Island, N.Y.) and additives: 5 ng/ml human recombinant epidermal growth factor (Upstate Biotechnology, Lake Placid, N.Y.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 ug/ml transferrin (Sigma, St. Louis, Mo.), 20 pM triiodothyronine

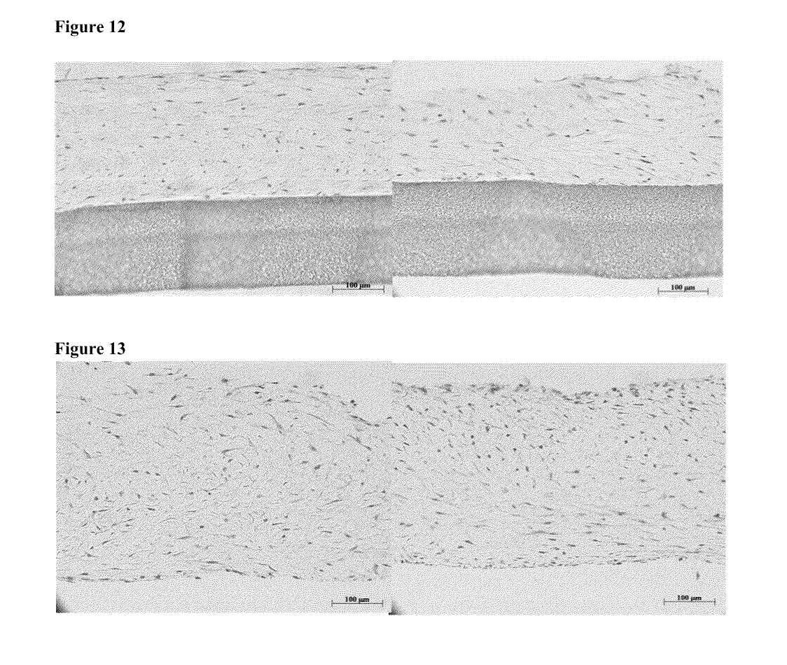

(Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 ug/ml L-proline (Sigma, St. Louis, Mo.), 0.1 ug/ml glycine (Sigma, St. Louis, Mo.), 20 ng/ml TGF-alpha (i.e., 1× TGF-alpha) and 10 nM PGE₂. The cells were cultured in this manner for 18 days prior to harvesting the bioengineered constructs. In some embodiments, 2× TGF-alpha or more can be preferable. Several bioengineered constructs were immediately formalin-fixed for histology analysis so as to prevent natural contraction (FIG. 12), whereas the remaining bioengineered constructs were control contracted, as described further below.

Specifically, sterile forceps were used to detach the bioengineered constructs from the Transwell membrane such that they were left floating in the culture dish. In order to produce a porous bioengineered construct while still retaining strong mechanical properties, the bioengineered constructs were contracted in a controlled manner by returning the floating constructs to an incubator and allowing the bioengineered constructs to naturally contract for two hours. After two hours, the media was removed, rinsed in RODI water, and formalin-fixed for histology analysis (FIG. 13). Bioengineered constructs having undergone controlled contraction (FIG. 12) show an approximately 2-fold increase in average bioengineered construct thickness (e.g., 400-800 μm average thicknesses versus 200-300 μm average thicknesses) relative to those not having undergone controlled contraction (FIG. 13).

In another embodiment, after the two hours of floating incubation, the bioengineered constructs were subsequently soaked in 1 mM EDC solution at 4° C. overnight, though the construct could alternatively be soaked in 0.2 mM EDC, 0.5 mM EDC, 5 mM EDC, or 10 mM EDC in the culture dishes without straying from the scope of the present invention. After EDC cross-linking, the construct was rinsed with reverse osmosis deionized (RODI) water three times, drained and laid flat. After rinsing with RODI water, the bioengineered constructs were cooled down from room temperature (˜20° C.) at a rate of 0.5° C. per minute for 2 hours until a final freezing temperature of −40° C. was reached. After the bioengineered construct reached a temperature of −40° C., the bioengineered construct was annealed at −40° C. for at least 2 hours. All bioengineered constructs were then subjected to a vacuum environment of lower than 200 mTorr in a lyophilizing apparatus and treated for twenty four hours at 0° C. It is to be appreciated that the freezing cycle can be performed in an appropriately-enabled lyophilizing apparatus or in any freezer, such as a control-rate freezer. It is to be further appreciated that the bioengineered constructs can be subjected to a vacuum environment between 0 mTorr and 350 mTorr without straying from the scope of the invention. In an alternative embodiment, theconstruct was allowed to air dry for 8 hours after EDC cross-linking without undergoing lyophilization (i.e., freeze drying).

In another embodiment, after the two hours of floating incubation, the media was removed, and the bioengineered constructs were rinsed in MES buffer until the constructs no longer had a pink color. The constructs were then soaked in reverse osmosis deionized (RODI) water for about one hour before being drained and laid flat. After rinsing with RODI water, the bioengineered constructs were cooled down from room temperature (˜20° C.) at a rate of 0.5° C. per minute for 2 hours until a final freezing temperature of −40° C. was reached. After the bioengineered construct reached a temperature of −40° C., the bioengineered construct was annealed at −40° C. for 2 hours. All constructs were then subjected to a vacuum environment of lower than 200 mTorr in a lyophilizing apparatus for twenty four hours at 0° C. The bioengineered constructs were subsequently placed in a vacuum oven for 24 hours at 100° C. to form dehydrothermal crosslinks (DHT) in the bioengineered constructs. In some embodiments, lyophilization can be preferred in the absence of crosslinking steps.

### Example 24

### Bioengineered Constructs have in Vivo Osteogenic and Barrier Function

Bioengineered constructs as those produced using the methods of Example 23 (i.e., EDC crosslinked, DHT crosslinked, and uncrosslinked bioengineered constructs, collectively referred to in this Example as the “test constructs”) in addition to a negative control (no construct) and a positive control (a 25×25 mm standard bioabsorbable barrier membrane of Bioguide, which comprises a porcine type I and III collagen membrane from Osteohealth, One Luitpold Drive, P.O. Box 9001, Shirley, N.Y. 11967) were implanted into each of the four quandrants of the jaw of Gottingen minipigs (maxillary right, maxillary left, mandibular right and mandibular left).

Specifically, Four male adult minipigs were co-housed in a separate room throughout the study at a temperature of 22+/−2° C. Each pig was anesthetized for 8 hours, during which time all bony defects were prepared and treated. The surgical procedure for applying each construct took approximately 2 hours. The second and fourth premolar teeth were extracted after 1) elevation of a full thickness gingival flap, 2) separation of the roots using a multiblade bur, and 3) incision of the periodontal ligament with an Orban scalpel. Before extractions, the buccal plate of the alveolar bone surrounding the teeth were penetrated with a round bur at various points and were cut using a carbide fissure bur by connecting the round bur holes. The buccal plate was surgically removed using bone chisels and bone scissors to create bone defects (1.2 cm² each). All constructs were 25×25 mm sections and were placed in randomly selected 4 maxillary and 4 mandibular sites so as to extend the mesial, distal and apical borders of the defect by 2-3 mm. Ligatures were used to tie the construct borders to the surrounding host gingival soft tissue. All surgical procedures were performed in aseptic conditions and using general anesthesia and endotracheal intubation provided by LASC veterinary services.

After 4, 8, and 12 weeks, the designated animals were sacrificed and the test/control sites were retrieved together with the adjacent bone in block sections and fixed in a 10% formalin solution. Half of the block sections at each group were decalcified using a decalcification agent. After decalcification and dehydration, the blocks were immersed in paraffin, and subsequently 5 micrometer sections were cut and stained with hematoxylin-eosin for light microscopy and identification of the cellular composition of inflammatory infiltratem as well as for histopathologic and histomorphologic examination. Sections were also stained with masson's trichrome to detect new collagen deposition and new bone formation. The other half of the block sections were fixed in 4% formalin solution after scraping away the overlying soft tissue, dehydrated in ascending grades of alcohol, and embedded in methylmethacrylate for future staining with toluidine blue for evaluation of new bone and collagen deposits. Alveolar bone structure and newly formed tissue compositions were examined by quantitative microcomputed tomography (MicroCT) following defect treatment. MicroCT Scans were performed using a Scanco microCT 80 system (Scanco Medical, Bassersdorf, Switzerland) located at the Boston University Orthopaedic and Development Biomechanics laboratory at the Department of Mechanical Engineering. Immediately before scanning, the jaws of the 4 minipigs were removed from storage and allowed to calibrate to room temperature.

The test sites treated with test constructs showed higher cell activity and turnover of newly formed tissues, i.e., connective and osteoid tissues). At 8 weeks, healthy connective tissue and highly organized newly formed osteoid tissue filled the defect areas and the contours of buccal bone were almost fully reformed. At 12 weeks, the test sites treated with test constructs showed almost complete healing with well-connected new bone formation with the old bone while some sections showed continued healing with some osteoclasts at the bone surface indicating bone turnover.

### Example 25

### Controlling Pore Size of Bioengineered Constructs

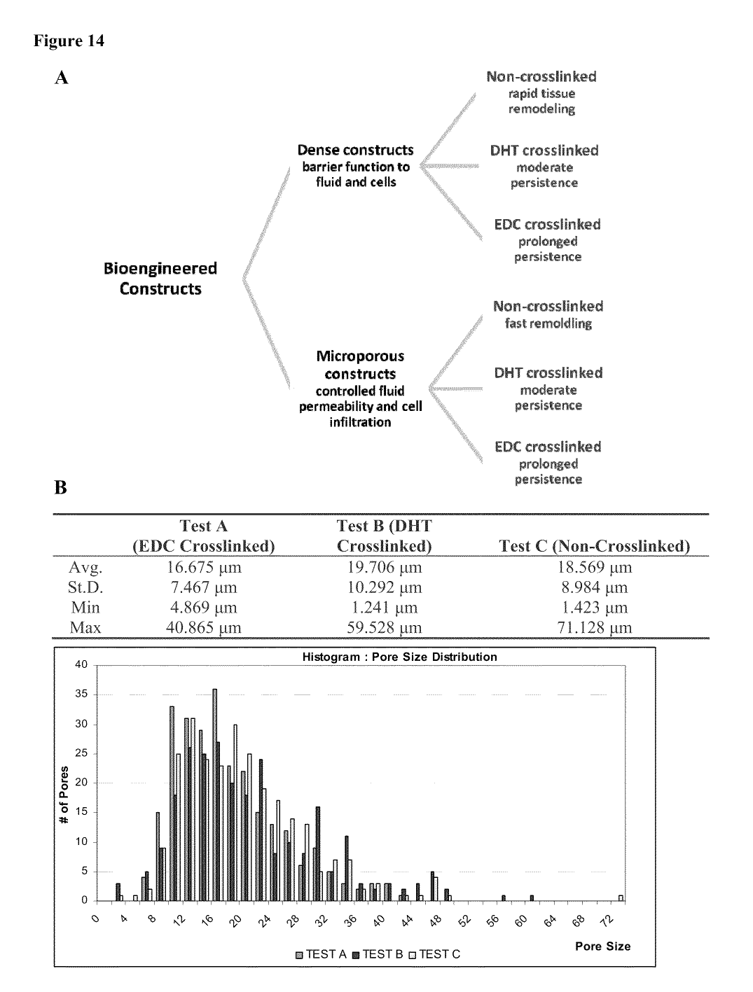

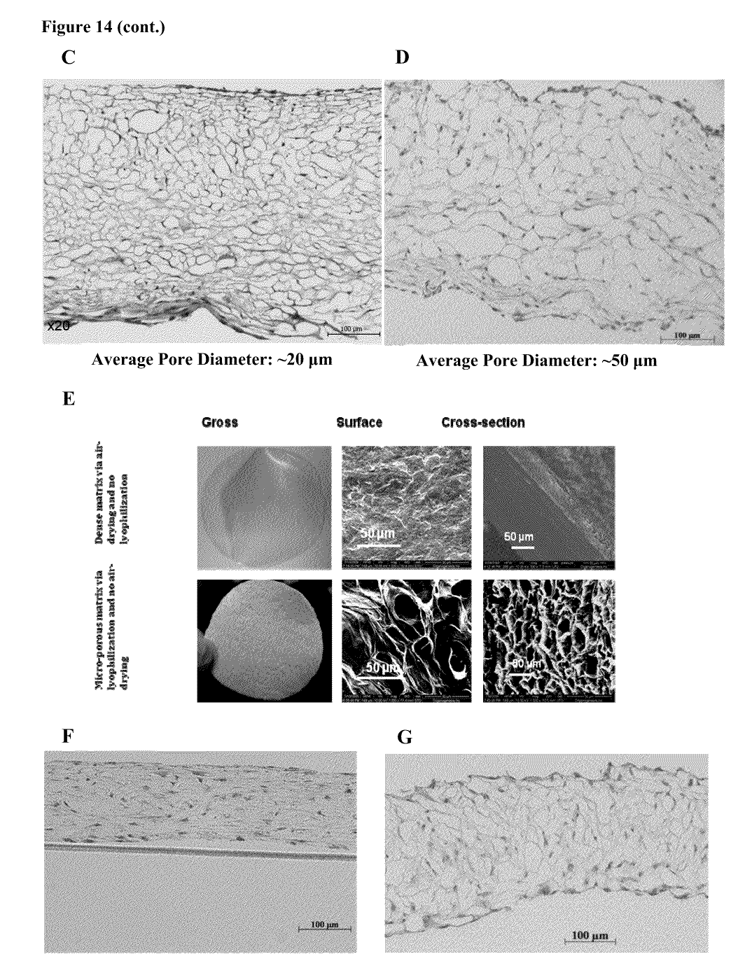

The average pore size within the extracellular matrix of the bioengineered constructs of the invention can be engineered to form dense or porous extracellular matrix. Combined with a type and/or degree of crosslinking, defined average pore sizes can be chosen and controlled to yield constructs that have different rates of in vivo persistence and/or cell infiltration, ranging from “rapidly bioremodelable” to “moderately bioremodelable” to “prolonged bioremodelable” bioengineered constructs for tailored applicability to therapeutic uses (FIG. 14A). HDF-derived bioengineered constructs produced according to the methods of Example 23, were analyzed after 18 days in culture to determine the pore size and distribution characteristics. FIG. 13 demonstrates that such bioengineered constructs that have not been lyophilized have essentially no pores. However, bioengineered constructs were further subjected to controlled contraction, lyophilization, and either not crosslinked, crosslinked with EDC, or crosslinked using DHT methods according the methods of Example 23. The magic wand tool of the Scandium® image analysis program (Olympus) was used to statistically analyze pores lengths and areas on representative histological sections. Since pores are not precise circles, pore diameter was back calculated assuming the measured area of a given pore was derived from a circle. Two histology images per group were used to generate the measurements. FIG. 14B shows that ramping to a final freezing temperature of −40° C., at a rate of 0.5° C. per minute, resulted in average pore sizes of between 15 and 20 μm. In addition, FIG. 14C further demonstrates that average pore size is determined by the final freezing temperature irrespective of crosslinking status. By contrast, FIG. 14D shows that ramping the bioengineered constructs to a final freezing temperature of −10° C., which is a warmer freezing temperature than −40° C., at a rate of 0.5° C. per minute, resulted in average pore sizes of at least 50 μm (e.g., ranging between 30 μm and 100 μm). FIG. 14E further demonstrate that the average pore size is independent of controlled contraction. Specifically, HDF-derived bioengineered constructs produced according to the methods of Example 23, and which were simply air dried after controlled contraction, produced a dense matrix with very small pores, if any). By contrast, bioengineered constructs that were processed as those shown in FIG. 14B produced average pore sizes of between 15 and 20 μm. Similarly, the average pore size of MSC-derived bioengineered constructs generated according to the methods of Example 19 (FIG. 14F) can be increased upon controlled contraction, rinsing, freezing from room temperature to −20° C., and lyophilization (FIG. 14G).

### Example 26

### Controlling Bioengineered Construct Thickness and ECM Composition

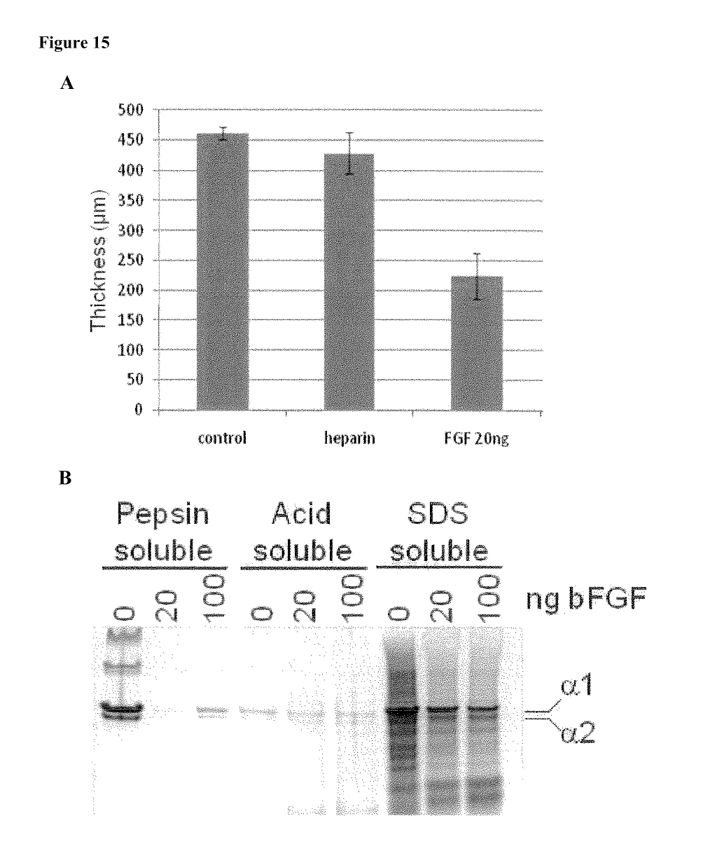

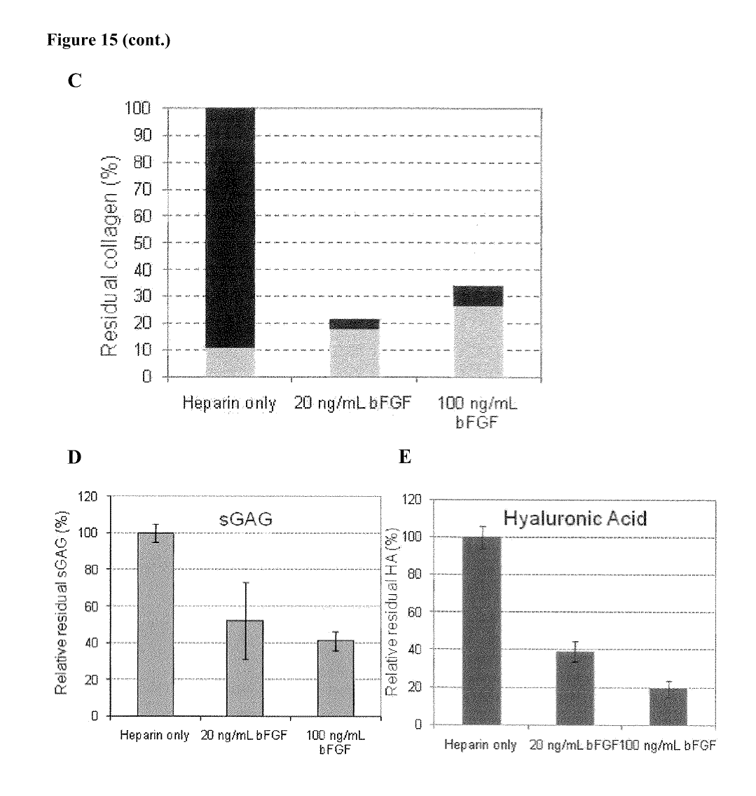

HDFs were seeded at superconfluency (i.e., 30×10⁶ cells per 75 mm insert) and cultured for 18 days according to the methods of Example 19, except that 20 ng/mL TGF-alpha was used. Heparin was also supplemented in the media at 5 μg/mL. To test the effect of basic fibroblast growth factor (bFGF; Peprotech Inc.) on the resulting bioengineered constructs, bFGF was supplemented and maintained in the culture media either upon initial seeding or after 5 days in culture. FIG. 15A shows that supplementing the chemically defined culture media with 20 ng/mL bFGF significantly reduced bioengineered construct thicknesses that were more easily tearable when handled with forceps relative to controls. Heparin supplementation had no effect on bioengineered construct thicknesses. Bioengineered constructs produced using 2 ng/mL bFGF had thicknesses similar to untreated controls.

The thinner bFGF-supplemented bioengineered constructs indicated that the extracellular matrix contained less matrix protein, less glycosaminoglycans, or both. FIG. 15B shows the results of bFGF dose response analysis in which collagen accumulation decreased as bFGF supplementation increased. Since collagen populations form sequentially during extracellular matrix production (i.e., reversibly crosslinked acid-soluble collagen, then pepsin-soluble collagen that is irreversibly crosslinked and must be isolated by cutting off crosslinks with pepsin, and then SDS soluble collagen, which is highly crosslinked and is neither acid-nor pepsin-soluble), each of these populations collagen populations were extracted from control and bFGF-supplemented bioengineered constructs using standard techniques. The total collagen accumulation in the bFGF-supplemented bioengineered constructs is lower relative to controls and there is an especially significant deficiency in the accumulation of pepsin soluble collagen (FIG. 15B). Heparin alone did not affect collagen accumulation.

Acid- and pepsin-soluble collagen amounts were independently assayed and quantified using a Sircol collagen assay on the bioengineered constructs analyzed in FIG. 15B. Since SDS-soluble collagen is not triple helical, the Sircol assay does not detect this class of collagen. FIG. 15C shows relative levels of both acid- and pepsin-soluble collagen (black) relative to total collagen and other collagen (grey). The combined amount of acid- and pepsin-soluble collagen in bioengineered constructs supplemented with 20 ng/mL or 100 ng/mL bFGF was 20% and 35%, respectively, of control amounts.

Differential scanning calorimetry (DSC) was subsequently performed to determine the total number of protein crosslinks in the bFGF-supplemented bioengineered constructs relative to controls. The peak area in bioengineered constructs supplemented with bFGF at either seeding or after 5 days in culture was decreased or zero relative to controls supplemented with heparin alone, indicating fewer crosslinks in the bFGF-supplemented bioengineered constructs.

In addition to changes in collagen amounts, sulfated glycosaminoglycan (sGAG), which are responsible for binding growth factors and help regulate ECM hydration, as well as hyaluronic acid (HA), accumulated to lower levels in bFGF-supplemented bioengineered constructs relative to controls (FIGS. 15D and 15E). Histological staining assays independently confirmed that the bFGF-supplemented bioengineered constructs were less dense, contained less sGAG (Alcian blue staining), and contained fewer elastic fibers (van Gieson staining).

The alterations in extracellular matrix composition caused the bFGF-supplemented bioengineered constructs to turn to powder when dehydrated indicating that such constructs could easily be micronized by grinding. Bioengineered constructs produced using 20 ng/mL cracked when lyophilized in a temperature-controlled freeze dryer cracked during lyophilization, but the fragments remained as pliable as control units. However, the fragments were also less thick and significantly more porous than the control units. Immediately prior to lyophilization, the bFGF-supplemented bioengineered constructs were placed in a −80° C. freezer for 2 hours. It is to be appreciated that the bFGF-supplemented bioengineered constructs could be kept in a freezer ranging in temperature from −10° C. to −80° C. anywhere from 1 hour to 3 days without straying from the scope of the invention. Alternatively, the bFGF-supplemented bioengineered constructs can be taken out of culture and placed directly into the lyophilizer. All bFGF-supplemented bioengineered constructs were then subjected to a vacuum environment of lower than 200 mTorr in a lyophilizing apparatus and treated for twenty four hours at 0° C. It is to be appreciated that the bioengineered constructs can be subjected to a vacuum environment between 0 mTorr and 350 mTorr without straying from the scope of the invention. In another embodiment, the bFGF-supplemented bioengineered constructs can be air-dried overnight at room temperature instead of treated in a lyophilizer.

The air-dried powder or lyophilized bFGF-supplemented bioengineered constructs, as well as controls, were micronized by grinding either using a mortar and pestle at room temperature or a tissue mill in which the constructs were kept frozen in liquid nitrogen. Similar amounts of ground constructs were rehydrated in phosphate buffered saline (PBS) in a microcentrifuge tube for 10 minutes prior to observation of fluid consistency. Rehydrated bFGF-supplemented constructs were significantly less viscous and floated more freely than control samples. This translated into an enhanced ability of the rehydrated bFGF-supplemented constructs to pass through a syringe needle (i.e., they could pass through 23 gauge and 27 gauge, but not 30 gauge needles, whereas controls could not pass through any such gauged syringe needles). Since scanning electron microscopy at 1000× magnification have determined that the particles in ground bFGF-supplemented constructs relative to controls are similar in size, it is believed that the viscosity of control particles impedes their passage through syringe needles. It is further believed that a finer or more consistent particle size can be achieved using finer tissue mills such that the rehydrated bFGF-supplemented constructs can pass through even finger gauged syringe needles.

### Example 27

### Porous Silk Scaffolds for Use with Bioengineered Constructs

Porous silk-based scaffolds were fabricated from degummed silk fiber of a Bombyx mori silkworm cocoon. The silk fibers were dissolved in 9 M LiBr solution at 6-10 wt % concentration for 6-10 hours while stirring under room conditions. The solution was dialyzed against water using a cellulose dialysis membrane for 3 days, changing the water every 10 hours. The fibroin aqueous solution was concentrated by standing the solution in a cellulose dialysis membrane. Insoluble portions were removed by centrifugation at 20,000 rpm for 30 minutes. The final concentration of the silk solution was about 7.5-8%.

The silk stock solution was then used to prepare a silk working solution with a concentration of 6% to 8%. The working solution was used to make a porous silk scaffold. The working solution was initially mixed with 1-6% ethanol solution with various volume ratios to make the final silk concentrations ranging from 3% to 5% and ethanol final concentrations ranging from 0.5% to 2%. The mixture was subsequently poured into a petri dish and placed into a −20° C. freezer for at least 10 hours. After the 10 hours has passed, the silk solution was placed at room temperature and allowed to thaw, resulting in a porous silk scaffold. The thawed silk scaffolds were subsequently rinsed in RODI water for 3 days to remove solvent residue. After rinsing, a top thin layer can be removed from the surface of the scaffolds. Silk scaffolds can be sterilized by autoclaving the final scaffold, or using autoclaved silk solution mixed with sterile filtered ethanol solution, or using sterile filtered silk solution mixed with sterile filtered ethanol solution.

In order to enhance blood vessel formation in vivo, porous silk scaffolds can be soaked in proteins such as platelet-derived growth factor (PDGF), fibroblast growth factor (FGF), hepatocyte growth factor/scatter factor (HGF/SF), insulin-like growth factor (IGF), vascular endothelial growth factor (VEGF) and other kinds of pro-angiogenic factors. In one aspect, 50 micrograms of recombinant human PDGF-BB powder was reconstituted in 0.5 ml 4 mM HCl, and then added with an additional 0.5 ml phosphate buffered saline (PBS). The resulting 1 mL solution was used to soak a 6×6 mm silk scaffold prior to implantation in a full thickness wound in nude and normal mice. In addition, 50 micrograms of recombinant human basic fibroblast growth factor (bFGF) was reconstituted in 1 mL of PBS. 6×6 mm porous silk scaffolds were soaked in the 1 mL bFGF solution for 5 minutes prior to implantation in a full thickness wound in nude and normal mice. Also, 50 micrograms of recombinant human PDGF-BB was reconstituted in 0.5 ml 4 mM HCL and subsequently mixed with 0.5 mL PBS-reconstituted recombinant human bFGF. Porous silk scaffolds were soaked in the resulting 1 mL solution for five minutes prior to implantation in a full thickness wound on nude and normal mice. In addition, silk scaffolds can be cultured with cells in chemically defined culture media comprising supplementation with 25 ng/ml of PDGF at day 5, 25 ng/ml of bFGF at day 10, and 25 ng/ml of hepatocyte growth factor (HGF) at day 15. Alternatively, the chemically defined cultured media comprises supplementation with 25 ng/ml of bFGF at day 5, 25 ng/ml of PDGF at day 10, and 25 ng/ml of bFGF at day 15 or 25 ng/ml of pDGF on day 5, 25 ng/ml bFGF on day 10, and 25 ng/ml of HGF on day 15. Also, the conditioned culture medium applied to bioengineered constructs on day 11 of Example 28 can be concentrated (e.g. 100-fold) and silk scaffolds can be soaked in the conditioned media.

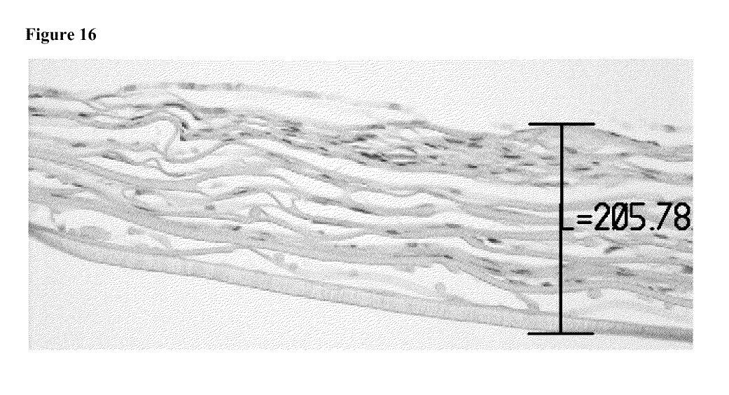

In one embodiment, human dermal fibroblasts were seeded onto the porous silk scaffold. Specifically, human dermal fibroblasts were initially seeded at about 30×10⁶ and cultured in chemically defined medium for 11 days. Alternatively, it is to be appreciated that HDFs can be seeded atop the silk scaffold at an initial seeding density of about 5×10⁶. The chemically defined medium comprised: a base 3:1 mixture of DMEM, Hams F-12 medium (Quality Biologics, Gaithersburg, Md.), 4 mM GlutaMAX (Gibco BRL, grand Island, N.Y.) and additives: 5 ng/ml human recombinant epidermal growth factor (Upstate Biotechnology, Lake Placid, N.Y.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 ug/ml transferrin (Sigma, St. Louis, Mo.), 13.5 pg/mL triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 ug/ml L-proline (Sigma, St. Louis, Mo.), 0.1 ug/ml glycine (Sigma, St. Louis, Mo.), 20 ng/ml TGF-alpha and 10 nM PGE₂. As can be seen in FIG. 16, the human dermal fibroblasts were able to migrate through the silk scaffolds and are uniformly disposed throughout the silk sheet.

Several modifications can be made to engineer desired characteristics into the resulting bioengineered constructs cultured on porous silk scaffolds.

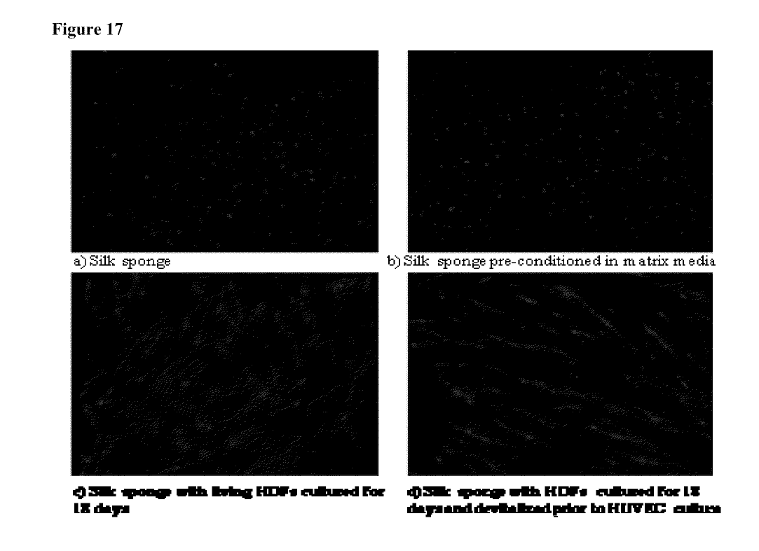

In another embodiment, the silk scaffolds having cultured HDFs were devitalized by rinsing the silk scaffolds comprising cultured HDFs with WFI water. For indications that require an enhanced angiogenic response, silk scaffolds having an average pore diameter of 50-100 microns, seeded with HDFs, and resulting in WFI water-devitalized bioengineered constructs have been shown to be an effective treatment. More specifically, FIG. 17(d) shows stained human umbilical vein endothelial cells atop the silk scaffolds with devitalized fibroblasts in vitro. The stained endothelial cells form aligned tubules atop the silk scaffolds, an indication that the silk scaffolds with devitalized fibroblasts allow for effective endothelial cell attachment and persistence.

In another embodiment, the bioengineered constructs containing porous silk scaffolds and devitalized HDFs were subsequently crosslinked with EDC in order to make a bioengineered tissue construct with enhanced in vivo persistence (e.g., in a burn wound bed).

Silk scaffolds can also be impregnated with useful molecules. Silk scaffolds were submerged in pre-conditioned, chemically defined culture medium previously collected (post-culture) from endogenously produced bioengineered tissue constructs to enhance the silk scaffolds. More specifically, about 30 million human dermal fibroblasts were cultured atop a 0.4 micrometer porous membrane and cultured in chemically defined medium for 11 days. The chemically defined medium comprises: a base 3:1 mixture of DMEM, Hams F-12 medium (Quality Biologics, Gaithersburg, Md.), 4 mM GlutaMAX (Gibco BRL, grand Island, N.Y.) and additives: 5 ng/ml human recombinant epidermal growth factor (Upstate Biotechnology, Lake Placid, N.Y.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 ug/ml transferrin (Sigma, St. Louis, Mo.), 13.5 pM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 ug/ml L-proline (Sigma, St. Louis, Mo.), 0.1 ug/ml glycine (Sigma, St. Louis, Mo.), 20 ng/ml TGF-alpha and 10 nM PGE₂. After the 11 days in culture, the conditioned medium was collected, and the silk scaffolds were soaked in the conditioned medium for 12 hours.

A silicone backing can also be applied to one or both sides of a silk scaffold to act as a barrier to preventing infection while allowing transport of gaseous molecules, such as oxygen. For example, silk scaffolds with devitalized human dermal fibroblasts were treated with a silicone coating. The silicone coating was optimized by varying the monomer concentration to crosslinker concentration ratio during polymerization of silicone. The ratio of the monomer to the crosslinker can range from about 5 to 1 to about 20 to 1. For a wet silk sponge, the optimal monomer to crosslinker ratio is about 5 to 1. In addition, the produced bioengineered construct can itself subsequently be coated with a silicone backing

Enhancement of epithelial cell migration can be achieved by bathing the silk scaffolds in a solution of phosphate buffered saline and laminin 5 for about 1 hour. Depending on the porosity profile of the silk scaffold, the scaffold can be submerged in the laminin 5 solution for up to 4 hours. The silk scaffold with conjugated laminin 5 can be employed in vivo to enhance epithelial cell migration.

### Example 28

### Layered Constructs of HDFs and MSCs

Human neonatal foreskin fibroblasts (originated at Organogenesis, Inc. Canton, Mass.) were seeded at 5×10⁵ cells/162 cm² tissue culture treated flask (Costar Corp., Cambridge, Mass., cat #3150) and grown in culture medium. The growth medium consisted of: Dulbecco's Modified Eagle's medium (DMEM) (high glucose formulation, without L-glutamine, BioWhittaker, Walkersville, Md.) supplemented with 10% newborn calf serum (NBCS) (HyClone Laboratories, Inc., Logan, Utah) and 4 mM L-glutamine (BioWhittaker, Walkersville, Md.). The cells were maintained in an incubator at 37±1° C. with an atmosphere of 10±1% CO₂. The medium was replaced with freshly prepared medium every two to three days. After 8 days in culture, the cells had grown to confluence, that is, the cells had formed a packed monolayer along the bottom of the tissue culture flask, and the medium was aspirated from the culture flask. To rinse the monolayer, sterile-filtered phosphate buffered saline was added to the bottom of each culture flask and then aspirated from the flasks. Cells were released from the flask by adding 5 mL trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the suspension to stop the action of the trypsin-versene. The cell suspension was removed from the flasks and evenly divided between sterile, conical centrifuge tubes. Cells were collected by centrifugation at approximately 800-1000×g for 5 minutes.

Cells were resuspended using fresh medium to a concentration of 3.0×10⁶ cells/ml, and seeded onto 0.4 micron pore size, 24 mm diameter tissue culture treated inserts (TRANSWELL®, Corning Costar) in a six-well tray at a density of 1.0×10⁶ cells/insert. It is to be appreciated that if a 75 mm insert is to be used, a cell seeding density of 10×10⁶ cells should be employed. If a 24 mm diameter insert is used, about 1×10⁶ cells/24 mm insert should be employed. It is to be appreciated that the amount of HUCPVC were added to the suspension as a percentage of the amount of fibroblasts. For example, to make a layered 24 mm construct containing 50% HUCPVC, 5×10⁵ HUCPVC were seeded atop the 1.0×10⁶ human neonatal foreskin fibroblasts previously seeded atop the porous membrane. Both fibroblasts and HUCPVC were submerged in 3 ml of matrix production media, which comprises:

| Component | Concentration | |
|---|---|---|
| DMEM | 96.0% | |
| L-Glutamine | 1060 | mg/L |
| Hydrocortisone | 0.4 | mg/L |
| Selenious acid | 6.78 | μg/L |
| Ethanolamine | 0.1 | mM |
| o-Phosphorylethanolamine | 14.0 | Mg/L |
| EGF | 10.0 | μg/L |
| Mg Ascorbate | 50 | mg/L |
| L-Proline | 213.6 | mg/L |
| Glycine | 101.4 | mg/L |
| Long TGFα | 10.0 | μg/L |

The cells were maintained in an incubator at 37±1° C. with an atmosphere of 10±1% CO₂ and cultured in the matrix production medium for 11 days with media changes made periodically, every 3-4 days.

The formalin fixed samples were embedded in paraffin and 5 micrometer sections were punched and then stained with hematoxylin-eosin (H&E) according to procedures known in the art. Using H&E stained slides, thickness measurements were made to ten randomly picked microscopic fields utilizing a 10X eyepiece loaded with a 10 mm/100 micrometer reticle.

### Example 29

### Production of Bioengineered Constructs by Admixing HDFs and MSCs

A construct having a fibroblasts and HUCPVC-produced extracellular matrix layer were formed in a fully chemically defined culture media system. 1×10⁵ human neonatal dermal fibroblasts are seeded in a mixed cell population with 9×10⁵ mesenchymal progenitor cells on a 24 mm culture insert. It is to be appreciated that the initial seeding density of the fibroblasts can range from about 1×10⁵ to about 9×10⁵ and the initial seeding density of the mesenchymal progenitor cells can also range from about 1×10⁵ to about 9×10⁵ within the scope of the invention. HUCPVC were obtained at passage 2, and expanded to passage 7 prior to being initially seeded upon the culture insert. It is to be appreciated that the HUCPVC can be used at any other passage number so long as the multipotentiality of the cells is preserved.

The chemically defined matrix production medium contained:

| Component | Concentration | |
|---|---|---|
| DMEM | 96.0% | |
| L-Glutamine | 1060 | mg/L |
| Hydrocortisone | 0.4 | mg/L |
| Selenious acid | 6.78 | μg/L |
| Ethanolamine | 0.1 | mM |
| o-Phosphorylethanolamine | 14.0 | Mg/L |
| EGF | 10.0 | μg/L |
| Mg Ascorbate | 50 | mg/L |
| L-Proline | 213.6 | mg/L |
| Glycine | 101.4 | mg/L |
| Long TGFα | 10.0 | μg/L |

Fibroblasts and mesenchymal progenitor cells were cultured in the matrix production medium for 11 days with media changes made periodically, every 3-4 days, resulting in an endogenously produced extracellular matrix.

### Example 30

### Production of an Epidermal Layer on Bioengineered Constructs

Human epidermal progenitor cells (HEP's; keratinocytes) are seeded atop the bioengineered constructs described in any one of Examples 19-26. HEP's are seeded after the bioengineered constructs have been in culture for about 11 days. A seeding density of about 3.5×10⁵-1.2×10⁶ cells/construct is preferred, however other initial seeding densities are also contemplated in accordance with the invention. At day 11, the skin-constructs with the HEP's are treated with a medium containing approximately:

| Component | Concentration | |
|---|---|---|
| DMEM:HAM's F-12 3:1 | 96.10% | |
| L-Glutamine | 1060 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| o-Phosphorylethanolamine | 14.0 | Mg/L |
| Selenious acid | 6.78 | μg/L |
| Adenine | 24.4 | mg/L |
| Mg Ascorbate | 50.0 | mg/L |
| Progesterone | 0.63 | μg/L |
| EGF | 10.0 | μg/L |
| Long TGFα | 10.0 | μg/L |

| | | |
|---|---|---|
| Lipid Concentrate | Arachidonic Acid | 0.004 mg/L |
| | Cholesterol | 0.220 mg/L |
| | DL-α-Tocopherol- | 0.140 mg/L |
| | Acetate | |
| | Linoleic Acid | 0.020 mg |
| | Linolenic Acid | 0.020 mg/L |
| | Myristic Acid | 0.020 mg/L |
| | Oleic Acid | 0.020 mg/L |
| | Palmitoleic Acid | 0.020 mg/L |
| | Palmitic Acid | 0.020 mg/L |
| | Pluronic ® F-68 | 200.0 mg/L |
| | Stearic Acid | 0.020 mg/L |
| | Tween ® 80 | 4.4 mg/L |

At day 13, differentiation of the HEP's are induced by using a differentiation medium containing the following:

| Component | Concentration | |
|---|---|---|
| DMEM:HAM's F-12 3:1 | 96.3% | |
| L-Glutamine | 1060 | mg/L |
| Hydrocortisone | 0.40 | mg/L |
| Insulin | 5.0 | mg/L |
| Transferrin | 5.0 | mg/L |
| Triiodothyronine | 13.5 | ng/L |
| Selenious acid | 0.00678 | mg/L |
| Ethanolamine | 0.1 | mM |
| o-Phosphorylethanolamine | 14.0 | Mg/L |
| Adenine | 24.4 | mg/L |
| Mg Ascorbate | 50.0 | mg/L |
| Progesterone | 0.63 | μg/L |
| CaCl2 | 265 | mg/L |

| | | |
|---|---|---|
| Lipid Concentrate | Arachidonic Acid | 0.004 mg/L |
| | Cholesterol | 0.220 mg/L |
| | DL-α-Tocopherol- | 0.140 mg/L |
| | Acetate | |
| | Linoleic Acid | 0.020 mg/L |
| | Linolenic Acid | 0.020 mg/L |
| | Myristic Acid | 0.020 mg/L |
| | Oleic Acid | 0.020 mg/L |
| | Palmitoleic Acid | 0.020 mg/L |
| | Palmitic Acid | 0.020 mg/L |
| | Pluronic ® F-68 | 200.0 mg/L |
| | Stearic Acid | 0.020 mg/L |
| | Tween ® 80 | 4.4 mg/L |

At day 15, the medium formulation is changed to induce cornification of the developing epithelial layer in a medium containing approximately:

| Component | Concentration | |
|---|---|---|
| DMEM | 48.0% | |
| HAM's F-12 | 48.0% | |
| L-Glutamine | 658 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| o-Phosphorylethanolamine | 14.0 | Mg/L |
| Selenius acid | 6.78 | μg/L |
| Adenine | 24.4 | mg/L |
| Mg Ascorbate | 50.0 | mg/L |
| Long TGFα | 10.0 | μg/L |

| | | | |
|---|---|---|---|
| MEM Non-Essential Amino Acid | L-Alanine | 1.78 | mg/L |
| Solution | L-Asparagine | 2.6 | mg/L |
| | L-Aspartic Acid | 2.66 | mg/L |
| | L-Glutamic Acid | 2.94 | mg/L |
| | Glycine | 1.5 | mg/L |
| | L-Proline | 2.3 | mg/L |
| | L-Serine | 2.1 | mg/L |
| MEM Vitamin Solution | NaCl | 17 | mg/L |
| | D-Ca | 0.2 | mg/L |
| | Pantothenate | | |
| | Choline | 0.2 | mg/L |
| | Chloride | | |
| | Folic Acid | 0.2 | mg/L |
| | i-Inositol | 0.4 | mg/L |
| | Nicotinamide | 0.2 | mg/L |
| | Pyridoxal HCl | 0.2 | mg/L |
| | Riboflavin | 0.020 | mg/L |
| | Thiamine HCl | 0.2 | mg/L |
| Lipid Concentrate | Arachidonic | 0.004 | mg/L |
| | Acid | | |
| | Cholesterol | 0.220 | mg/L |
| | DL-α- | 0.140 | mg/L |
| | Tocopherol- | | |
| | Acetate | | |
| | Linoleic Acid | 0.020 | mg/L |
| | Linolenic Acid | 0.020 | mg/L |
| | Myristic Acid | 0.020 | mg/L |
| | Oleic Acid | 0.020 | mg/L |
| | Palmitoleic Acid | 0.020 | mg/L |
| | Palmitic Acid | 0.020 | mg/L |
| | Pluronic ® F-68 | 200.0 | mg/L |
| | Stearic Acid | 0.020 | mg/L |
| | Tween ® 80 | .4 | mg/L |

Cornfication medium is changed every 2-3 days. Bioengineered constructs are matured and maintained during days 22 through 35 and are fed a maintenance medium with changes every 2-3 days with fresh maintenance medium containing:

| Component | Concentration | |
|---|---|---|
| DMEM | 48.0% | |
| HAM's F-12 | 48.0% | |
| L-Glutamine | 658 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| O-phosphorylethanolamine | 14.0 | Mg/L |
| Selenius acid | 6.78 | μg/L |
| Adenine | 24.4 | mg/L |
| Long TGFα | 10.0 | μg/L |

| | | | |
|---|---|---|---|
| MEM Non-Essential Amino Acid | L-Alanine | 1.78 | mg/L |
| Solution | L-Asparagine | 2.64 | mg/L |
| | L-Aspartic Acid | 2.66 | mg/L |
| | L-Glutamic Acid | 2.94 | mg/L |
| | Glycine | 1.5 | mg/L |
| | L-Proline | 2.3 | mg/L |
| | L-Serine | 2.1 | mg/L |
| MEM Vitamin Solution | NaCl | 17 | mg/L |
| | D-Ca | 0.2 | mg/L |
| | Pantothenate | | |
| | Choline | 0.2 | mg/L |
| | Chloride | | |
| | Folic Acid | 0.2 | mg/L |
| | i-Inositol | 0.4 | mg/L |
| | Nicotinamide | 0.2 | mg/L |
| | Pyridoxal HCl | 0.2 | mg/L |
| | Riboflavin | 0.020 | mg/L |
| | Thiamine HCl | 0.2 | mg/L |
| Lipid Concentrate | Arachidonic | 0.004 | mg/L |
| | Acid | | |
| | Cholesterol | 0.220 | mg/L |
| | DL-α- | 0.140 | mg/L |
| | Tocopherol- | | |
| | Acetate | | |
| | Linoleic Acid | 0.020 | mg/L |
| | Linolenic Acid | 0.020 | mg/L |
| | Myristic Acid | 0.020 | mg/L |
| | Oleic Acid | 0.020 | mg/L |
| | Palmitoleic Acid | 0.020 | mg/L |
| | Palmitic Acid | 0.020 | mg/L |
| | Pluronic ® F-68 | 200.0 | mg/L |
| | Stearic Acid | 0.020 | mg/L |
| | Tween ® 80 | .4 | mg/L |

When the bioengineered constructs are fully formed, the cultured bioengineered constructs exhibit a mixed bioengineered layer of endogenously produced extracellular matrix proteins, fibroblasts and/or mesenchymal progenitor cells with a differentiated epithelial layer disposed atop the bioengineered construct.

### Example 31

### Etching of Bioengineered Tissue Construct to Improve Cell Infiltration

Bioengineered tissue constructs can be modified to enhance cell attachment and cell infiltration within the deep network of pores on endogenously produced tissue constructs. Such endogenously produced constructs can be produced by initially seeding about 30 million human dermal fibroblasts atop a 0.4 micrometer porous membrane and cultured in chemically defined medium for 11 days. The chemically defined medium comprises: a base 3:1 mixture of DMEM, Hams F-12 medium (Quality Biologics, Gaithersburg, Md.), 4 mM GlutaMAX (Gibco BRL, grand Island, N.Y.) and additives: 5 ng/ml human recombinant epidermal growth factor (Upstate Biotechnology, Lake Placid, N.Y.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 ug/ml transferrin (Sigma, St. Louis, Mo.), 20 pM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 ug/ml L-proline (Sigma, St. Louis, Mo.), 0.1 ug/ml glycine (Sigma, St. Louis, Mo.), 20 ng/ml TGF-alpha and 10 nM PGE₂. After the 11 days of culture, the surface of the bioengineered tissue constructs can be etched to remove cell debris. This can be done by applying a solution of 1% acetic acid in order to remove a thin layer of collagen from the top surface of the bioengineered construct. Etching can allow for improved cell infiltration, which can be advantageous in a burn indication.

Although the foregoing invention has been described in some detail by way of illustration and Examples for purposes of clarity and understanding, it will be obvious to one of skill in the art that certain changes and modifications may be practiced within the scope of the appended claims.

We claim:

1. A bioengineered construct comprising a devitalized layer of extracellular matrix produced and assembled by cultured extracellular matrix producing cells.

2. The bioengineered construct of claim 1, further comprising one or more additional devitalized layers of extracellular matrix produced and assembled by cultured extracellular matrix producing cells.

3. The bioengineered construct of claim 2, wherein the layers of extracellular matrix have been decellularized of the cultured extracellular matrix producing cells.

4. The bioengineered construct of claim 3, wherein the layers of extracellular matrix adjacently contact each other at a bonding region.

5. The bioengineered construct of claim 4, wherein the layers of extracellular matrix in adjacent contact at the bonding region are bonded together by crosslinks.

6. The bioengineered construct of claim 4, wherein the layers of extracellular matrix in adjacent contact at the bonding region are bonded together by a bioremodelable or bioresorbable adhesive disposed between said layers.

7. The bioengineered construct of claim 6, wherein the bioremodelable or bioresorbable adhesive is a solution derived from Bombyx mori silkworm.

8. The bioengineered construct of claim 7, wherein the solution comprises silk fibroin at a concentration between about 2% to about 8% w/v.

9. The bioengineered construct of claim 2, wherein at least one layer of extracellular matrix is crosslinked.

10. The bioengineered construct of claim 2, wherein at least one layer of extracellular matrix is crosslinked to a lesser degree and at least one layer of extracellular matrix is crosslinked to a higher degree.

11. The bioengineered construct of claim 1, wherein the layer is produced in conditions that include a chemically defined culture medium containing no animal-derived components.

12. The bioengineered construct of claim 1, wherein the extracellular matrix producing cells are derived from tissue selected from the group consisting of neonate male foreskin, dermis, tendon, lung, urethra, umbilical cord, corneal stroma, oral mucosa, and intestine.

13. The bioengineered construct of claim 1, wherein the extracellular matrix producing cells are derived from stem cells.

14. The bioengineered tissue construct of claim 1, wherein the cultured extracellular matrix producing cells are dermal fibroblasts.

15. A method for making a bioengineered construct, comprising:
culturing extracellular matrix producing cells in a first culture under conditions that induce the cells to form a first layer of extracellular matrix;
culturing extracellular matrix producing cells in a second culture under conditions that induce the cells to form a second layer of extracellular matrix;
terminating the extracellular matrix producing cells in both the first layer of extracellular matrix and second layer of extracellular matrix to form first and second devitalized layers of extracellular matrix;
contacting the first devitalized layer of extracellular matrix to the second devitalized layer of extracellular matrix by superimposing said first and second layers to form a bonding region;
bonding said first and second layers, wherein said bonding is achieved by crosslinking or an adhesive.
